(19) United States
(12) Patent Application Publication
(10) Pub. No.: US 2025/0041340 A1
(43) Pub. Date: Feb. 6, 2025

(54) A NOVEL METHOD OF TREATING TARGETED ABNORMAL CELLS, AND CYTOTOXIC CELL USED THEREIN

(71) Applicant: Acepodia Biotechnologies Ltd., Taipei City (TW)

(72) Inventors: CHING-WEN HSIAO, Castro Valley, CA (US); ZIH-FEI CHENG, Taipei City (TW); TAI-SHENG WU, Taipei City (TW); YAN-LIANG LIN, Taipei City (TW); HAO-KANG LI, Taipei City (TW); SAI-WEN TANG, San Mateo, CA (US); HSIU-PING YANG, Taipei City (TW); SHIH-CHIA HSIAO, Taipei City (TW)

(73) Assignee: Acepodia Biotechnologies Ltd., Taipei City (TW)

(21) Appl. No.: 18/717,216

(22) PCT Filed: Dec. 12, 2022
(86) PCT No.: PCT/US2022/081368

§ 371 Date: Jun. 6, 2024

Related U.S. Application Data
(60) Provisional application No. 63/288,728, filed on Dec. 13, 2021.

(51) Int. Cl. A61K 35/17; A61P 35/00; C07K 16/28; C07K 16/30; C12N 5/0783

(52) U.S. Cl. CPC A61K 35/17; A61P 35/00; C07K 16/2863; C07K 16/30; C12N 5/0646; C07K 2317/732

(57) ABSTRACT

The present invention provides a method of treating targeted abnormal cells that are resistant, refractory, insensitive, non-responsive, or inadequately responsive to an ingredient, as well as cytotoxic cells used therein, comprising administering an effective amount of the ingredient-complexed cytotoxic cells to a subject with the disease.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 12, 2022, is named “7171-0108PWO1.xml” and is 45,853 bytes in size. The sequence listing contained in this .XML file is part of the specification and is hereby incorporated by reference herein in its entirety.

### FIELD OF THE INVENTION

This present invention relates to a method of treating targeted abnormal cells that are resistant, refractory, insensitive, non-responsive, or inadequately responsive to an ingredient, as well as cytotoxic cells used therein; more particularly relates to a method of treating targeted abnormal cells (including but not limited to resistant cells) that are resistant, refractory, insensitive, non-responsive, or inadequately responsive to an ingredient, as well as the cytotoxic cells complexed with the ingredient used therein.

### BACKGROUND OF THE INVENTION

Cytotoxic cells play a role against infectious or tumour invasion. There are two types of cytotoxic cells: those involved in innate or natural immunity such as natural killer cells, gamma delta T cells, and NKT cells; and those involved in adaptive or acquired immunity such as cytotoxic T lymphocytes (Rey et al., 2005.)

Therapy targeting abnormal cells is a treatment that uses ingredients designed to aim at abnormal cells having unique biological markers without affecting other cells. Therapy targeting abnormal cells such as targeted cancer therapy has been developed over the past decades (Lupu et al., 2016). However, it still faces many challenges. The first main challenge is that there are few drugs available for therapy targeting abnormal cells. In fact, many ingredients such as codrituzumab, solanezumab, bimagrumab, tralokinumab, and bococizumab, that show great potential to specifically interact with (or bind to) biological markers expressed on the abnormal cells were successful in the Phase I trial, but they were determined to be ineffective or without adequate efficacy in Phase II/III trials. In other word, the drug development process could not be completed and therefore all of these numerous ingredients with great potential could not be used in the industry.

The second main challenge is drug resistance. For example, a Food and Drug Administration-approved drug (FDA-approved drug) such as trastuzumab or cetuximab may be effective when it is first administered to patients, killing targeted abnormal cells and reducing the number of the targeted abnormal cells. However, after a period of time, the efficiency of the same drug at killing the targeted abnormal cells has proven to be lowered drastically (Lee M. Ellis and Daniel J. Hicklin, 2009.)

In order to solve drug resistance problem, people have tried to improve their understanding of drug resistance as well as the diseases to develop novel therapy targeting abnormal cells based on other ingredients or other unique biological features since 2012 (Bottsford-Miller et al., 2012). However, developing a new drug from an original idea to a finished product is a complex process, which can take up 12-15 years and cost in excess of $1 billion (Hughes et al., 2011), and most of the ingredients showing great potential would often fail in the clinical trials. Moreover, even if one of the potential ingredients would be successful passed in the drug development process and then approved by the Food and Drug Administration (FDA), abnormal cells may still develop resistance against this new drug.

Consequently, there is still an urgent need for a method of solving the problem of few curative drugs available for therapy targeting abnormal cells in the market as well as solving the drug resistance problem. The inventors of the present invention believe that cytotoxic cells have the potential to solve these problems and be applied in cell therapy that specifically targets abnormal cell.

In addition, current effective therapy is limited in most patients by a key barrier known as “the immunosuppressive microenvironment” (David H. Munn and Vincenzo Bronte, 2016). Therefore, there is still an urgent need for a method of solving the problem of few curative drugs available for treating abnormal cells located in the immunosuppressive microenvironment.

### SUMMARY OF THE INVENTION

The purpose of the present invention is to provide an in vitro or in vivo method of treating abnormal cells, more particularly for treating abnormal cells that are resistant, refractory, insensitive, non-responsive, or inadequately responsive to current FDA-approved drugs.

The second purpose of the present invention is to provide novel cytotoxic cells that are capable of treating abnormal cells, more particularly for treating abnormal cells that are resistant, refractory, insensitive, non-responsive, or inadequately responsive to current FDA-approved drugs.

The third purpose of the present invention is to provide a method of increasing the migratory capacity of immune cells into a lesion comprising abnormal cells; more particularly into a lesion comprising abnormal cells that are resistant, refractory, insensitive, non-responsive, or inadequately responsive to current FDA-approved drugs.

The fourth purpose of the present invention is to provide novel cytotoxic cells that are capable of increasing the migratory capacity of immune cells into a lesion comprising abnormal cells; more particularly into a lesion comprising abnormal cells that are resistant, refractory, insensitive, non-responsive, or inadequately responsive to current FDA-approved drugs.

The fifth purpose of the present invention is to provide cytotoxic cells conjugated with an ingredient based on chemical conjugation technology, wherein the ingredient is ineffective or without adequate efficacy in treating a disease or in treating a subject with a disease.

The sixth purpose of the present invention is to provide a method to improve the effectiveness of treatment in a subject who is resistant, refractory, insensitive, non-responsive, or inadequately responsive to current FDA-approved drugs.

The seventh purpose of the present invention is to provide a method to improve the effectiveness of ingredients that were successful in the Phase I trial but failed in Phase II or Phase III trials.

In a first aspect, the present invention provides a method of treating a disease, comprising administering an effective amount of effector cells to a subject with the disease; the effector cells comprise a surface and a population of targeting units complexed to the surface of the effector cells; wherein a targeting unit in the population comprises a first ingredient; and the first ingredient is characterized in that: (a) it exhibits specific interaction with a biological marker expressed by abnormal cells associated with the disease; (b) it is not produced by the effector cell; and (c) it is determined to be ineffective or without adequate efficacy in treating the subject with the disease or concluded to be ineffective or without adequate efficacy in treating the disease at the end of a clinical trial.

In a second aspect, the present invention further provides a method of increasing the migratory capacity of immune cells into lesion of a disease, comprising administering an effective amount of effector cells to a subject with the disease; the effector cells comprise a surface and a population of targeting units complexed to the surface of the effector cells; wherein a targeting unit in the population comprises a first ingredient, and the first ingredient is characterized in that: (a) it exhibits specific interaction with a biological marker expressed by abnormal cells located in lesion of the disease; (b) it is not produced by the effector cell; and (c) it is determined to be ineffective or without adequate efficacy in treating the subject with the disease or concluded to be ineffective or without adequate efficacy in treating the disease at the end of a clinical trial.

In some embodiments, the method is for increasing the migratory capacity of immune cells into lesion of the subject with the disease.

In a third aspect, The present invention further provides an in vitro method of reducing the number of abnormal cells associated with a disease, comprising contacting a plurality of abnormal cells associated with the disease with an effective amount of effector cells; the effector cells comprise a surface and a population of targeting units complexed to the surface of the effector cells; wherein a targeting unit in the population comprises a first ingredient; and the first ingredient is characterized in that: (a) it exhibits specific interaction with a biological marker expressed by the abnormal cells associated with the disease; (b) it is not produced by the effector cell; and (c) it is determined to be ineffective or without adequate efficacy in treating the abnormal cells associated with the disease.

In some embodiments, the effector cells are cytotoxic cells.

In some embodiments, about or more than 5%, 10%, 15%, 20%, 25%, 30%, 35%, 40%, 45%, 50%, 55%, 60%, 65%, 70%, 75%, 80%, 85%, 90%, 95%, 99%, or 99.5% of the cells to be administered to the subject are the effector cells comprising the population of targeting units complexed to the surface of the effector cells. In some embodiments, all cells to be administered to the subject are the effector cells comprising the population of targeting units complexed to the surface of the effector cells.

In some embodiments, the subject is a vertebrate. Preferably, the subject is a mammal selected from the group consisting of murines, simians, humans, farm animals, sport animals, pets, and other mammals. Preferably, the subject is a human.

In some embodiments, the effector cells comprise more than 3000 targeting units per cell. In some embodiments, each of the targeting units in the population comprises at least one first ingredient.

In some embodiments, the effector cells comprise at least 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, 40000, 60000, 80000, 100000, 110000, 120000, 130000, 140000, 150000, 170000, 190000, 210000, 220000, 230000, 240000, 250000, 260000, 270000, 280000, 290000, 300000, 320000, 340000, 360000, 380000, 400000 targeting units per cell.

In some embodiments, the effector cells are CD16⁺ cells.

In some embodiments, the first ingredient comprises an Fc receptor recognized region.

In some embodiments, the first ingredient is an antibody. Preferably, the first ingredient is a monoclonal antibody of an IgG subtype that induces ADCC; or the first ingredient is other antibody; or the first ingredient comprises an antigen-binding unit.

In some embodiments, the Fc receptor recognized region is an Fc region of an antibody.

In some embodiments, the antibody is HER1, HER2, HER3, HER4, EGFR, CD20, CD19, ErbB2, ErbB3, CD28, IGF1R, IMC-1121, Met, VEGF, PDGFRα, PDGFRβ, CD22, CD79b, CD32B, IGF-1, IGF-1R, IGF-2, OPN, Ang-2, VEGFR, EpCAM, ROR1, PD-L1, VEGFR1, VEGFR2, or VEGFR3. In some embodiments, the effector cell is complexed to antibodies directed to one, two, or more of: HER1, HER2, HER3, HER4, EGFR, CD20, CD19, ErbB2, ErbB3, CD28, IGF1R, IMC-1121, Met, VEGF, PDGFRα, PDGFRβ, CD22, CD79b, CD32B, IGF-1, IGF-1R, IGF-2, OPN, Ang-2, VEGFR, EpCAM, ROR1, PD-L1, VEGFR1, VEGFR2, VEGFR3 (see U.S. Pat. No, 10,744,207).

In some embodiments, the antibody is a polyclonal antibody, monoclonal antibody, chimeric antibody, humanized antibody, or fully human antibody.

In some embodiments, the first ingredient is not a nucleic acid.

In some embodiments, the effector cells are capable of mediating an antibody-dependent cell cytotoxicity (ADCC) response.

In some embodiments, the effector cells are capable of inducing the migration of CD3⁺ T cells.

In some embodiments, after co-cultured with target cells expressing the biological marker, the effector cells express CD107a.

In some embodiments, after co-cultured with target cells expressing the biological marker, the effector cells express interferon-γ (IFN-γ) and/or Tumor Necrosis Factor-α (TNF-α).

In some embodiments, the surface is an outer surface of the effector cell. In some embodiments, the surface is an outer surface of the cell membrane of the effector cell. In some embodiments, the surface is the outer surface of the membrane structure of the effector cell.

In some embodiments, the target cells are Raji, Daudi, K562, other target cells associated with liquid tumor, A549, SK-OV-3, BT-474, other target cells associated with solid tumor, or other target cells associated with the disease.

In some embodiments, the first ingredient is an FDA-approved ingredient for the treatment of the disease.

In some embodiments, the first ingredient is rituximab, trastuzumab, cetuximab, alemtuzumab, avelumab, daratumumab, elotuzumab, obinutuzumab, vorsetuzumab, cusatuzumab, durvalumab, panitumumab, or amatuzimab.

In some embodiments, the first ingredient has been successful in phase I clinical trial but is not an FDA-approved ingredient for the treatment of the disease.

In some embodiments, “first ingredient that is successful in phase I clinical trial but is not an FDA-approved ingredient for the treatment of the disease” is an ingredient that was successful in the Phase I trial but is not approved by FDA for the treatment of the disease because the ingredient is determined to be ineffective or without adequate efficacy in Phase II/III trials. For example, codrituzumab is a first ingredient that is successful in phase I clinical trial but is not an FDA-approved ingredient for the treatment of the disease.

In some embodiments, the first ingredient is codrituzumab, solanezumab, bimagrumab, traklokinumab, or bococizumab.

In some embodiments, the effector cells to be administered to the subject are derived from autologous effector cells or allogeneic effector cells.

In some embodiments, the effector cells are autologous effector cells. In some other embodiments, the effector cells are derived from an allogeneic subject and therefore are allogeneic effector cells. With autologous cell therapies, patients receive the autologous effector cells derived from their own body. With allogeneic cell therapy, patients receive the allogeneic effector cell derived from a donor who is not the patient (the allogeneic subject).

In some embodiments, the method comprises (a) obtaining a population of autologous effector cells or allogeneic effector cells; (b) complexing one or more first ingredients to the autologous effector cells or allogeneic effector cells; and (c) administering the complexed cells from step (b) to the subject thereby treating the disease in the subject. In some embodiments, steps (a) to (c) are completed within 24 hours. In some embodiments, the complexed cells (effector cells) from step (b) are administered without inducing cell expansion prior to administration.

In some embodiments, the disease is selected from the group consisting of hyperproliferative diseases, advanced stage disease, HIV or other viral infectious diseases, fungi infectious diseases, bacteria infectious diseases, protozoan infectious diseases, autoimmune diseases, neuronal diseases, hematopoietic cell-related diseases, metabolic syndromes, and pathogenic diseases.

In some embodiments, the disease is a hyperproliferative or advanced stage disease selected from the group consisting of solid tumors and liquid tumors.

In some embodiments, the hyperproliferative disease is at least one of cancers, solid tumors or liquid tumors include Acanthoma, Acinic cell carcinoma, Acoustic neuroma, Acral lentiginous melanoma, Acrospiroma, Acute cosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adeno carcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richters transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma (see U.S. Pat. No, 10,744,207).

In some embodiments, the HIV or other viral infectious diseases are caused by virus infection. Examples of infectious virus to which stimulation of a protective immune response is desirable include: Retroviridae (e.g., human immunodeficiency viruses, such as HIV-1 (also referred to as HTLV-111, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvo viruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astro viruses), bacteria infectious diseases are caused by bacteria infection. Examples of infectious bacteria to which stimulation of a protective immune response is desirable include: Helicobacter pylons, Borellia burgdorferi, Legionella pneumophila, Mycobacteria sps (e.g. M. tuberculosis, M. avium, M. intracellulare, M. kansaii, M. gordonac), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridians group), Streptococcus faecalis, Streptococcus bovis, Streptococcus (anaerobic sps.), Streptococcus pneumonia, pathogenic Campylobacter sp., Enterococcus sp., Haemophilus influenza, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumonia, Pasteurella multocida, Bacteroides sp., Fusobacterium nucleatum, Streptobacillus monïliformis, Treponema pallidum, Treponema pertenue, Leptospira, and Actinomyces israelii. Examples of infectious fungi to which stimulation of a protective immune response is desirable including: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium falciparum and Toxoplasma gondii (see U.S. Pat. No, 10,744,207).

In some embodiments, the first ingredient exhibits specific interaction with a biological marker selected from the group consisting of cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, and MAGE, or any combination thereof; or the first ingredient exhibits specific interaction with a cancer antigen selected from the group consisting of HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), ROR1, ROR2, PD-1 (CD279), CTLA-4 (CD152), TIM-3 (HAVCR2), an immune checkpoint receptor, an immune checkpoint receptor ligand, a receptor tyrosine kinase-like orphan receptor, a TNF receptor protein, an immunoglobulin protein, a cytokine receptor, an integrin, and activating NK cell receptors, or any combinations thereof.

In some embodiments, the targeting unit is complexed to the surface of the effector cell via an interaction between a first linker conjugated to the first ingredient and a second linker conjugated to the surface of the effector cell.

In some embodiments, the first linker is covalently or non-covalently conjugated to the first ingredient; or the second linker is covalently or non-covalently conjugated to the surface of the effector cell; or a combination thereof.

In some embodiments, the first linker or the second linker is conjugated to a native functional group of the first ingredient or the surface of the effector cell, wherein the native functional group is an amino acid, a sugar, or an amine.

In some embodiments, the native functional group comprises a sugar, an amine, a or an amino acid; or wherein the native functional group is not an azide-modified sugar such as N-a-azidoacetyl sialic acid (SiaNAz); or wherein the native functional group comprises an amino acid selected from the group consisting of lysine, cysteine, tyrosine, threonine, serine, aspartic acid, glutamic acid and tryptophan (see U.S. Pat. No, 10,744,207).

In some embodiments, the second linker is directly, covalently linked to the native functional group of the effector cell; wherein the direct, covalent link between the second linker and the native functional group of effector cell is prepared by contacting the effector cell with the second linker, such that the second linker is directly, covalently linked to the native functional group (see U.S. Pat. No, 10,744,207).

In some embodiments, the first linker and the second linker are selected from the group consisting of: a DNA binding domain and a target DNA; a leucine zipper and a target DNA; biotin and avidin; biotin and streptavidin; calmodulin-binding protein and calmodulin; a hormone and a hormone receptor, lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

In some embodiments, at least one of the two linkers comprises a PEG region or an NHS ester, or wherein the first ingredient is conjugated to the first linker via a coupling group, wherein the coupling group is an NHS ester or other activated ester, an alkyl or acyl halide, a bifunctional crosslinker, or maleimide group (see U.S. Pat. No, 10,744,207).

In some embodiments, the binding affinity between the first linker and the second linker is less than 250 nM. Preferably, the binding affinity between the first linker and the second linker is less than 200 nM, 100 nM, 50 nM, 10 nM, 1 nM, 500 pM, 100 pM, 60 pM, 50 pM, 20 pM, 15 pM, 10 pM, 5 pM, or 2 pM.

In some embodiments, the first ingredient and the effector cell are separated by a length of 1 nm to 400 nm. Preferably, the first ingredient and the effector cell am separated by a length of 1, 2, 5, 10, 15, 20, 30, 32, 35, 40, 45, 50, 60, 62, 65, 70, 75, 80, 90, 100, 120, 150, 180, 200, 220, 250, 280, 300, 320, 350, or 380 nm; or between 1 nm to 200 nm, 1 nm to 100 nm, 1 nm to 50 nm, 5 nm to 100 nm, or 5 nm to 50 nm.

In some embodiments, the targeting unit and the effector cell are separated by a length of 1 mu to 20 nm or 1 nm to 33 nm. Preferably, the targeting unit and the effector cell are separated by a length of 2, 3, 4, 5, 6, 7, 8, 9, 10, 12, 14, 16, 18, 20, 22, 24, 26, 28, 30, or 32 nm.

In some embodiments, the binding affinity of the first ingredient for the biological marker is less than 250 nM. Preferably, the binding affinity of the first ingredient for the biological marker is less than 200 nM, 100 nM, 50 nM, 10 nM, 1 nM, 500 pM, 100 pM, 60 pM, 50 pM, 20 pM, 15 pM, 10 pM, 5 pM, or 2 pM.

In some embodiments, the first linker is a first polynucleotide, and the second linker is a second polynucleotide.

In some embodiments, the first polynucleotide is a compound comprised of deoxyribonucleotides, ribonucleotides, or analogs thereof, or any combination thereof. Preferably, the Second polynucleotide is a compound comprised of deoxyribonucleotides, ribonucleotides, or analogs thereof, or any combination thereof (see U.S. Pat. No, 10,744,207).

In some embodiments, at least one of the two polynucleotides is a DNA, an RNA or a peptide nucleic acid (PNA) molecule, or any combination thereof (see U.S. Pat. No, 10,744,207).

In some embodiments, the length of at least one of the two polynucleotides is 4 nt to 500 nt.

In some embodiments, the length of at least one of the two polynucleotides is 5, 10, 15, 20, 25, 30, 35, 40, 45, 50, 55, 60, 65, 70, 75, 80, 85, 90, 95, 100, 120, 140, 160, 180, 200, 300, 400 or 500 nt.

In some embodiments, the first polynucleotide comprises a first single-stranded region, and the second polynucleotide comprises a second single-stranded region complementary to the first single-stranded region, wherein the targeting unit is complexed to the surface of the effector cell via the interaction between the first single-stranded region and the second single-stranded region complementary to the first single-stranded region (see U.S. Pat. No, 10,744,207).

In some embodiments, the first polynucleotide and the second polynucleotide are substantially or fully complementary to each other. For example, the two polynucleotides share at least about 10%, 20%, 30%, 40%, 50%, 60%, 70%, 80%, 90%, 956, 96%, 97%, 98%, 99% or 100% complementarity (see U.S. Pat. No, 10,744,207).

In some embodiments, the first polynucleotide or second polynucleotide comprise a sequence selected from 20-mer poly-GGTT, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, 23-mer SEQ ID NO: 25, and SEQ ID NO:26.

In some embodiments, linkers are designed to have about or less about 90%, 80%, 70%, 60%, 50% Y, 40%, 30%, 20%, 10%, or lower GC content. In some embodiments, the linkers arm selected to have about or more than about, 10%, 20%, 30%, 40%, 50% Y, 60%, 70%, 80%, 90%, or more GC content. In some embodiments, linkers am designed to comprise or consist of sequences of 1, 2, 3, 4, 5, 6, 7, 8, 9, or 10 nucleotides repeated 1, 2, 3, 4, 5, 6, 7, 8, 9, 10, 15, or 20 times, or repeated until reaching the end of the linker (e.g., AAA . . . , or ATAT . . . ). In some embodiments, linkers are selected to have a Tm of about or more than about 30° C., 35° C., 40° C., 45° C., 50° C., 55° C., 60° C., 65° C., 70° C., 75° C., 80° C., 85° C., or higher (see U.S. Pat. No, 10,744,207).

In some embodiments, the first linker comprises a first reactive group, and the second linker comprises a second reactive group, and wherein the targeting unit is complexed to the surface of the effector cell via a covalent bond formed by a reaction between the second reactive group and the first reactive group.

In some embodiments, the cytotoxic cell is an immune cell, a lymphocyte, a natural killer cell, a gamma delta T cell, other T lymphocytes, macrophages, monocytes, a neutrophil, dendritic cells, cytokine-induced killer cells (CIK), lymphokine-activated killer cells (LAK), cytolytic T cells (CTLs), or tumor-infiltrating lymphocytes (TIL).

In some embodiments, the cells such as the effector cells are derived from a cell line.

Examples of cell lines include, but ae not limited to Raw264.7, U-937, oNK, NK92, NK3.3, KHYG-1, NKL, and transgenic varieties thereof. Cell lines are available from a variety of sources known to those with skill in the art (see, e.g., the American Type Culture Collection (ATCC) (Manassas, Va.)) (see U.S. Pat. No. 10,744,207).

In some embodiments, the cell such as effector cell is a T cell. T cells include all cells which express CD3, including CD4⁺ T cells, CD8⁺ T cells, and CD4⁺CD8⁺ T cells. T cells include both naive and memory cells (e.g., T_{CM}, T_{EM} and T_{EMRA}), helper cells (e.g., Th1, Th2, T13, Th9, Th7, T_{FH}), effector cells (e.g., CTLs or Tc cells), NKT cells, tumor infiltrating lymphocytes (TILs), regulatory cells (e.g., Treg, and Tr1 cells), lymphocyte-activated killer cells (LAKs), αβT cells, γδT cells (gamma delta T cell), and similar unique classes of the T cell lineage. In some embodiments the T cells are activated T-cells (see U.S. Pat. No. 10,744,207).

In some embodiments, the cells such as effector cells are a population of cells enriched in Cytokine Induced Killer (CIK) cells. The methods for enriching CIK cells are described in U.S. Pat. No, 10,744,207, which is hereby incorporated by reference. In some embodiments, the cell such as effector cell is an embryonic stem cell (ESC), such as from an ESC cell line (see U.S. Pat. No, 10,744,207).

In some embodiments, the effector cell is non-tumorigenic in an immune compromised mouse; or the effector cell is non-tumorigenic in an allogeneic subject after being irradiated with γ-ray.

In some embodiments, the effector cell further comprises an inactive tumor suppressor gene or a mutated and highly expressed oncogene.

In some embodiments, the effector cell is stored at or below 0° C. prior to the administration.

In some embodiments, the effector cell is stored at or below −20° C., −80° C., −130° C., or −196° C. prior to the administration.

In some embodiments, the disease is cancer, and the abnormal cells associated with the disease are cancer cells. Preferably, the disease is tumor, and the abnormal cells associated with the disease are tumor cells. Preferably, the disease is bacteria infectious diseases, and the abnormal cells associated with the disease are bacteria-infected cells. Preferably, the disease is HIV or other viral infectious diseases, and the abnormal cells associated with the disease are HIV or other virus-infected cells. Preferably, the disease is fungi infectious diseases, and the abnormal cells associated with the disease are fungi-infected cells. Preferably, the disease is protozoan infectious diseases, and the abnormal cells associated with the disease are protozoan-infected cells. Preferably, the abnormal cells associated with the disease are healthy or abnormal cells located in an abnormal location associated with the disease. Preferably, the abnormal cells associated with the disease are healthy or abnormal cells located in a lesion of the disease. Preferably, the abnormal cells associated with the disease are the cells at pathological conditions located in a lesion of the disease.

In some embodiments, the cytotoxic cell is a natural killer cell characterized in that:
(A) it is deposited at NPMD having the deposit number NITE BP-03017; (B) it comprises a chromosome, and the chromosomal DNA sequence of the chromosome is at least 80% identical with the chromosomal DNA sequence of a corresponding chromosome of the natural killer cell deposited at NPMD having the deposit number NITE BP-03017; or (C) it has the following characteristics:
   i) expressing a CD16 receptor,
   ii) retaining its capability to proliferate after subculture for at least 3 months; and
   iii) x) not including synthetic, genetically modified and/or deliberately delivered polynucleotide encoding the CD16 receptor, or y) by using ddPCR system to analyze the genomic DNA of the cytotoxic cell, the ratio of CD16 F176F probe-detectable DNA molecule to CD16 F176V probe-detectable DNA molecule is equal to or higher than 1, wherein the sequence of the CD16 F176F probe is SEQ ID NO: 27 and the sequence of the CD16 F176V probe is SEQ ID NO: 28.

Preferably, the cytotoxic cell further characterized in that:
(1) the cytotoxic cell and the natural killer cell line NK3.3 are derived from different subjects;
(2) the cytotoxic cell is derived from a subject with a cancer,
(3) the cytotoxic cell is derived from a Caucasian male; or
(4) the cytotoxic cell and the natural killer cell having the deposit number ATCC CRL-2407 are derived from the same subject;
or any combination thereof.

In some embodiments, the cytotoxic cell is derived from the natural killer cell deposited at NPMD having the deposit number NITE BP-03017.

In some embodiments, the chromosomal DNA sequence of the chromosome in the cytotoxic cell is at least 85, 90, 95, 96, 97, 98, 99%, 99.99%, or 99.995% identical with the chromosomal DNA sequence of the corresponding chromosome of the natural killer cell deposited at NPMD having the deposit number NITE BP-03017.

In some embodiments, the chromosome is selected from the group consisting of: chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, the X chromosome, and the Y chromosome.

In some embodiment, the chromosome is the chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, the X chromosome, or the Y chromosome of the cytotoxic cell, and the corresponding chromosome is respectively the chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, the X chromosome, or the Y chromosome of the natural killer cell deposited at NPMD having the deposit number NITE BP-03017.

In some embodiments, a whole genome of the cytotoxic cell is at least 95%, 96%, 97%, 98%, 99%, 99.5%, 99.95%, 99.995%, or 99.9995 identical with the whole genome of the natural killer cell deposited at NPMD having the deposit number NITE BP-03017.

In some embodiments, the cytotoxic cell is capable of proliferating after subculture for at least 4 months, 5 months, 6 months, 7 months, 8 months, 9 months, 10 months, 11 months, 1 year, 2 years, 3 years, 4 years, 5 years, 6 years, 7 years, or 8 years.

In some embodiments, the effector cells further express CD2, CD45, CD4, CD25, NKp30, NKG2D, NKp44, NKp46, CD27, OX40, CD107a, NKG2A, PD-1, TIGIT, SIRPα, or CD158, or any combination thereof.

In some embodiments, the CD16 receptor is a CD16a receptor or a CD16b receptor.

In some embodiments, an expressed polynucleotide encoding the CD16 receptor is located on q arm of chromosome 1 at position 1q23.3.

In some embodiments, a polynucleotide encoding the CD16 receptor comprises a nucleotide sequence of SEQ ID NO: 29, SEQ ID NO: 30, or SEQ ID NO: 31.

In some embodiments, the CD16 receptor comprises an amino acid sequence of SEQ ID NO: 32, SEQ ID NO: 33, or SEQ ID NO: 34.

In some embodiments, the cytotoxic cell is a γδ T cell; or the cytotoxic cell is a Vδ1 T cell, a Vδ2 T cell, a Vδ3 T cell, a Vδ5 T cell, or a Vγ9Vδ2 T cell.

In some embodiments, the effector cells further express CD3, NKp46, CD56, CD16, NKG2D, NKp44, CD25, CD38, PD-1, NKp30, CD18, TIGIT, DNAM-1, CD36, CD103, CCR7, CXCR3, IFNγ, Granzyme B, or CD69, or any combination thereof.

In some embodiments, after co-culture with target cells, the effector cells further express Granzyme B.

In some embodiments, wherein:
(1) at least 4% of the effector cells express at least 400 NKp46 molecules per cell;
(2) at least 10% of the effector cells express at least 400 CD56 molecules per cell;
(3) at least 10% of the effector cells express at least 400 CD16 molecules per cell;
(4) at least 30% of the effector cells express at least 40 NKG2D molecules per cell;
(5) at least 1% of the effector cells express at least 400 NKp44 molecules per cell;
(6) at least 80% of the effector cells express at least 400 CD69 molecules per cell; or
(7) at lease 40% of the effector cells express at least 400 CXCR3 molecules per cell;
or any combination thereof.

In some embodiments, wherein:
(1) at least 4%, 5%, 6%, 7%, 8%, 9%, 10%, 15%, 20%, 25%, 30%, 35%, 40%, 45%, or 50% Y of the effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp46 molecules per cell;
(2) at least 10%, 15%, 20%, 25%, 30%, 35%, 40%, 45%, or 50% of the effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CD56 molecules per cell;
(3) at least 10%, 15%, 20%, 25%, 30%, 35%, 40%, 45%, or 50% of the effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CD16 molecules per cell;
(4) at least 30%, 35%, 40%, 45%, 50%, 55%, 60%, 65%, or 70% of the effector cells express at least 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, 700000, 710000, 720000, 730000, 740000, 750000, 760000, 770000, 780000, 790000, 800000, 810000, 820000, 830000, 840000, 850000, 860000, 870000, 880000, 890000, 900000, 910000, 920000, 930000, 940000, 950000, 960000, 970000, 980000, 990000, 1000000, 1250000, 1500000, 1750000, 2000000, 2250000, 2500000, 2750000, 3000000, 3250000, 3500000, 3750000, 4000000, 4250000, 4500000, 4750000, 5000000, 5250000, 5500000, 5750000, 6000000, 6250000, 6500000, 6750000, or 7000000 NKG2D molecules per cell;
(5) at least 1%, 2%, 3%, 4%, 5%, 6%, 7%, 8%, 9%, 10%, 15%, 20%, 25%, 30%, 35%, 40%, 45%, or 50% of the effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp44 molecules per cell;
(6) at least 80%, 81%, 82%, 83%, 84%, 85%, 86%, 87%, 88%, 89%, or 90% of the effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CD69 molecules per cell; or
(7) at least 50%, 55%, 60%, 63%, 67%, 70%, 73%, 77%, 80%, 83%, 87%, 90%, 93%, 97% or 90% of the effector cells express at least 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CXCR3 molecules per cell; or any combination thereof.

In some embodiments, wherein:
(1) at least 4% of the effector cells express NKp46, wherein the NKp46-expressing effector cells express at least 400 NKp46 molecules per cell on average;
(2) at least 10% of the effector cells express CD56, wherein the CD56-expressing effector cells express at least 400 CD56 molecules per cell on average;
(3) at least 10% of the effector cells express CD16, wherein the CD16-expressing effector cells express at least 400 CD16 molecules per cell on average;
(4) at least 30% of the effector cells express NKG2D, wherein the NKG2D-expressing effector cells express at least 40 NKG2D molecules per cell on average;
(5) at least 1% of the effector cells express NKp44, wherein the NKp44-expressing effector cells express at least 400 NKp44 molecules per cell on average;
(6) at least 80% of the effector cells express CD69, wherein the CD69-expressing effector cells express at least 400 CD69 molecules per cell on average; or
(7) at least 40% of the effector cells express CXCR3, wherein the CXCR3-expressing effector cells express at least 400 CXCR3 molecules per cell on average;
or any combination thereof.

In some embodiments, wherein:
(1) at least 4%, 5%, 6%, 7%, 8%, 9%, 10%, 15%, 20%, 25%, 30%, 35%, 40%, 45%, or 50% of the effector cells express NKp46, wherein the NKp46-expressing effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp46 molecules per cell on average;
(2) at least 10%, 15%, 20%, 25%, 30%, 35%, 40%, 45%, or 50% of the effector cells express CD56, wherein the CD56-expressing effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CD56 molecules per cell on average;
(3) at least 10%, 15%, 20%, 25%, 30%, 35%, 40%, 45%, or 50% of the effector cells express CD16, wherein the CD16-expressing effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CD16 molecules per cell on average;
(4) at least 30%, 35%, 40%, 45%, 50%, 55%, 60%, 65%, or 70% of the effector cells express NKG2D, wherein the NKG2D-expressing effector cells express at least 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, 700000, 710000, 720000, 730000, 740000, 750000, 760000, 770000, 780000, 790000, 800000, 810000, 820000, 830000, 840000, 850000, 860000, 870000, 880000, 890000, 900000, 910000, 920000, 930000, 940000, 950000, 960000, 970000, 980000, 990000, 1000000, 1250000, 1500000, 1750000, 2000000, 2250000, 2500000, 2750000, 3000000, 3250000, 3500000, 3750000, 4000000, 4250000, 4500000, 4750000, 5000000, 5250000, 5500000, 5750000, 6000000, 6250000, 6500000, 6750000, or 7000000 NKG2D molecules per cell on average;
(5) at least 1%, 2%, 3%, 4%, 5%, 6%, 7%, 8%, 9%, 10%, 15%, 20%, 25%, 30%, 35%, 40%, 45%, or 50% of the effector cells express NKp44, wherein the NKp44-expressing effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp44 molecules per cell on average;
(6) at least 80%, 81%, 82%, 83%, 84%, 85%, 86%, 87%, 88%, 89%, or 90%, of the effector cells express CD69, wherein the CD69-expressing effector cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CD69 molecules per cell on average; or
(7) at least 40%, 45%, 50%, 55%, 60%, 63%, 67%, 70%, 73%, 77%, 80%, 83%, 87%, 90%, 93%, 97% or 90%, of the effector cells express at least 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, or 700000 CXCR3 molecules per cell on average;
or any combination thereof.

In some embodiments, 10%˜90% of the effector cells am effector memory T cells (EM cells).

In some embodiments, 15%, 20%, 25%, 30%, 35%, 40%, 45%, 50%, 55%, 60%, 65%, 70%, 75%, 80% Y, 85%, 86%, 87%, 88%, or 89% of the effector cells are effector memory T cells (EM cells).

In some embodiments, 90%˜10% of the effector cells am terminally differentiated effector memory T cells (TDEM cells).

In some embodiments, 85%, 80% Y, 75%, 70%, 65%, 60%, 55%, 50% Y, 45%, 40%, 35%, 30%, 25%, 20% Y, 15% Y, 14%, 13%, 12%, or 11% of the effector cells am terminally differentiated effector memory T cells (TDEM cells).

In some embodiments, the targeting unit is a first type of targeting unit, and the effector cells further comprise a population of second type of targeting units complexed to the surface of the effector cells, wherein a targeting unit in the population of second type of targeting units comprises a second ingredient characterized in that (a) it exhibits specific binding to the biological marker or a different biological marker expressed by the abnormal cells associated with the disease; (b) it is not produced by the effector cell. In some embodiments, the second ingredient is an antibody. In some embodiments, the second ingredient is a monoclonal antibody of an IgG subtype that induces ADCC; or the second ingredient is other antibody; or the second ingredient comprises an antigen-binding unit. In some embodiments, the second ingredient is not a nucleic acid. In some embodiments, the second ingredient is an FDA-approved ingredient for the treatment of the disease. In some embodiments, the second ingredient is rituximab, trastuzumab, cetuximab, alemtuzumab, avelumab, daratumumab, elotuzumab, obinutuzumab, vorsetuzumab, cusatuzumab, durvalumab, panitumumab, or amatuzimab. In some embodiments, the second ingredient has been successful in phase I clinical trial but is not an FDA-approved ingredient for the treatment of the disease. In some embodiments, the second ingredient is codrituzumab, solanezumab, bimagrumab, tralokinumab, or bococizumab.

### BRIEF DESCRIPTION OF THE DRAWING

FIG. 1A is the bar chart presenting the comparison of the cytotoxic function between the trastuzumab-complexed human CD16⁺ natural killer cells and the non-complexed human CD16⁺ natural killer cells to kill trastuzumab-responsive cancer cells.

FIG. 1B is a bar chart presenting the comparison of the cytotoxic function between the trastuzumab-complexed human CD16⁺ natural killer cells and the non-complexed human CD16⁺ natural killer cells to kill trastuzumab-resistant cancer cells.

FIG. 2 is a bar chart presenting the cytotoxic effect of trastuzumab, human CD16⁺ natural killer cells, human CD16⁺ natural killer cells co-cultured with trastuzumab, and trastuzumab-complexed human CD16⁺ natural killer cells on trastuzumab-resistant cancer cells.

FIG. 3A-3B are histogram of gamma delta T cells collected on day 10.

FIG. 4A-4B are histogram of gamma delta T cells collected on day 14.

FIG. 5A is the standard curve of fluorescent dye PE-conjugated mouse anti-human CD56 derived from Quantum™ Simply Cellular® kit.

FIG. 5B is the standard curve of fluorescent dye PE-Cy7-conjugated mouse anti-human CD16⁺ derived from Quantum™ Simply Cellular® kit.

FIG. 5C is the standard curve of fluorescent dye PE-Cy7-conjugated mouse anti-human NKG2D derived from Quantum™ Simply Cellular® kit.

FIG. 5D is the standard curve of fluorescent dye PE-Cy7-conjugated mouse anti-human NKp44 derived from Quantum™ Simply Cellular® kit.

FIG. 5E is the standard curve of fluorescent dye PE-Cy7-conjugated mouse anti-human NKp46 derived from Quantum™ Simply Cellular® kit.

FIG. 6A is a bar chart presenting the comparison of the cytotoxic function between the trastuzumab-complexed human gamma delta T cells and the non-complexed human gamma delta T cells to kill normal cancer cells.

FIG. 6B is a bar chart presenting the comparison of the cytotoxic function between the trastuzumab-complexed human gamma delta T cells and the non-complexed human gamma delta T cells to kill trastuzumab-resistant cancer cells.

FIG. 7A is a bar chart presenting the comparison of the cytotoxic function between the cetuximab-complexed human gamma delta T cells and the non-complexed human gamma delta T cells to kill normal cancer cells.

FIG. 7B is a bar chart presenting the comparison of the cytotoxic function between the cetuximab-complexed human gamma delta T cells and the non-complexed human gamma delta T cells to kill cetuximab-resistant cancer cells.

FIG. 8A is a bar chart presenting the effect of hypoxia on the cytotoxic function of ingredient-complexed cytotoxic cells and the non-complexed human cytotoxic cells.

FIG. 8B is a bar chart presenting the effect of lactate (a metabolic waste) on the cytotoxic function of ingredient-complexed cytotoxic cells.

FIG. 8C is a bar chart presenting the effect of ascites extract (comprising immunosuppressive cytokines) on the cytotoxic function of ingredient-complexed human cytotoxic cells.

FIG. 9A illustrates the assay design of ingredient-complexed cytotoxic cell migration study.

FIG. 9B is a bar chart presenting the migratory capacity of the ingredient-complexed cytotoxic cells into a region where cancer cells are located.

FIG. 10A illustrates the assay design of CD3⁺ T cells migration study.

FIG. 10B is a bar chart presenting the effect of the ingredient-complexed cytotoxic cells on the migratory capacity of the CD3⁺ T cells into a region where cancer cells are located.

FIG. 11 is a bar chart presenting the comparison of the cytotoxic function between the human gamma delta T cells complexed with different number of trastuzumab to kill trastuzumab-resistant cancer cells.

FIG. 12 is a bar chart presenting the comparison of the cytotoxic function between the human gamma delta T cells complexed with different number of rituximab to kill rituximab-resistant cancer cells.

FIG. 13 is a bar chart presenting the comparison of the migratory capacity of the human gamma delta T cells complexed with different number of rituximab into a region where cancer cells are located.

FIG. 14 is the bar chart presenting the comparison of the cytotoxic function between the anti-GPC3 antibody-complexed human CD16⁺ natural killer cells and the non-complexed human CD16⁺ natural killer cells to kill cancer cells.

FIG. 15 is a bar chart presenting the comparison of the cytotoxic function between the anti-GPC3 antibody-complexed human gamma delta T cells and the non-complexed human gamma delta T cells to kill cancer cells.

### DETAILED DESCRIPTION OF THE INVENTION

The term “ingredient” such as “first ingredient” or “second ingredient” refers to any substance, molecule, compound, protein, cell, or active ingredient that confers some beneficial effect upon administration to a subject. The beneficial effect includes recognition of biological marker, recognition of abnormal cells; specific interaction with a biological marker expressed by abnormal cells; specific interaction with (or binding to) a biological marker expressed by abnormal cells; amelioration of a disease, symptom, disorder, or pathological condition; enablement of diagnostic determinations; generally counteracting a disease, symptom, disorder or pathological condition; and reducing or preventing the onset of a disease, symptom, disorder or condition. Examples of ingredients include, but are not limited to, cell, antibody, hormone or other ligand, lectin, carbohydrate, nucleic acid (RNA or DNA) hybridizing sequences, aptamer, other ingredients, and the like (see U.S. Pat. No, 10,744,207).

The term “effective amount” refers to the amount of an ingredient that is sufficient to affect beneficially or generate desired results. Other definitions for “effective amount” am well known to those skilled in the art such as described in textbooks or documents like U.S. Pat. No, 10,744,207, which am hereby incorporated by reference (see U.S. Pat. No, 10,744,207).

The term “FDA-approved ingredient for the treatment of the disease” refers to any ingredient that is approved by FDA for the treatment of the disease. For example, rituximab, trastuzumab, cetuximab, alemtuzumab, avelumab, daratumumab, and elotuzumab am FDA-approved ingredients for the treatment of different cancers or tumors.

The term “ingredient has been successful in phase I clinical trial but is not an FDA-approved ingredient for the treatment of the disease” refers to any ingredient that may show potential to treat the disease and has been successful in phase I clinical trial but is not an FDA-approved ingredient for the treatment of the disease because, for instance, the ingredient is determined to be ineffective or without adequate efficacy in Phase II/III trials. For example, codrituzumab is an ingredient that has been successful in phase I clinical trial but is not an FDA-approved ingredient for the treatment of the cancer or tumor.

The term “antigen-binding unit” refers generally to a moiety having a high affinity for an antigen. Non-limiting examples of antigen binding units include aptamers and antibodies (see U.S. Pat. No. 10,744,207). In some embodiment, some ingredients such as the first ingredient comprising an antigen-binding unit is an antibody comprising an Fc receptor recognized region, and therefore these ingredients could be recognized by an Fc receptor (for example recognized by Fc gamma receptors). In some embodiment, some ingredients such as the first ingredient comprising an antigen-binding unit is a monoclonal antibody of an IgG subtype.

The term “cytotoxic cells” refers to any cell has the ability to kill or destroy other living cells; for example, cytotoxic T cells, natural killer cells, gamma delta T cells, macrophages, or any other cells that has the ability to kill or destroy other living cells or mediates any cell cytotoxicity including, but not limited to, antibody-dependent cell cytotoxicity (ADCC).

The term “lesion of a disease” refers to any region in an organ or tissue which has suffered damage or abnormal change caused by or associated with a disease, for example, lesions of ovarian cancer.

The term “abnormal cells associated with a disease” refers generally to any cell, which has suffered abnormal change caused by or associated with a disease and therefore the cell has at least an abnormal characteristic such as abnormal expression of any molecules or is located in abnormal microenvironment.

The term “ingredient that is determined to be ineffective or without adequate efficacy in treating the subject with the disease” refers generally to any ingredient (such as a FDA-approved ingredient for the treatment of the disease) that is determined not effective or without adequate efficacy in treating the subject with the disease (prior to or after the administration of the ingredient) because, for instance, (1) the subject or the abnormal cell of the subject is non-responsive, inadequately responsive, insensitive, refractory, or resistant to the ingredient, or (2) on the abnormal cell of the subject, them are not enough biological markers that could be specifically recognized by the ingredient.

The term “ingredient that is concluded to be ineffective or without adequate efficacy in treating the disease at the end of a clinical trial” refers generally to any ingredient (such as an ingredient that has been successful in phase I clinical trial but is not an FDA-approved ingredient for the treatment of the disease) that is concluded or determined to be ineffective or without adequate efficacy in treating the disease in, for instance, Phase I/III trials.

The term “gdT cells”, “γδ T cells”, or “gamma delta T cells” refers to a subset of T cells that express a distinct chain combination of the T cell receptor (TCR), γδ TCR (rather than αβ TCR), on their surface, composed of a TCR-γ chain such as Vγ2, Vγ3, Vγ4, Vγ5, Vγ8, Vγ9, Vγ11 and a TCR-δ chain such as Vδ1, Vδ2, Vδ3, and Vδ5. The term “gdT cells” specifically includes all subsets of gdT cells and combinations thereof (see Pistoia et al., 2018; WO2020117862A1).

The interaction between the first linker and the second linker may be direct or indirect. In general, an indirect interaction is one that is mediated by one or more intermediate compounds. An intermediate compound may be of the same or different type as one or both linkers. In some embodiments, the first and second linkers are the same and interact via simultaneous interaction with an intermediate compound. For example, the first and second linkers may be the same antibody, which interact indirectly with one another by way of simultaneously binding an intermediate compound comprising two or more copies of the antigen to which the antibody is directed. In some embodiments, the first linker and the second linker are different. In some embodiments, the first and second linker interact directly. In general, a direct interaction is an interaction that does not require interaction with an intermediate compound. In some embodiments, the targeting unit comprises a targeting moiety conjugated to a first polynucleotide, and the therapeutic unit comprises a therapeutic agent conjugated to a second polynucleotide, and wherein said targeting unit and said therapeutic unit form a complex via complementarity between the first polynucleotide and the second polynucleotide, or with an adapter polynucleotide. The first and second polynucleotides may interact directly, such as by hybridizing to one another. The first and second polynucleotides may interact indirectly, such as via interaction with an intermediate compound. In some embodiments, the intermediate compound is an adapter polynucleotide, such as described herein. For example, the first and second polynucleotides may interact indirectly via complementarity with portions of the adapter polynucleotide. In some embodiments, the first and second linker are reactive groups that react with one another to form a covalent bond. Each reactive group may first be reacted directly with the entity to which it is attached (e.g., a targeting moiety or a therapeutic agent) to form a covalent bond (see U.S. Pat. No, 10,744,207).

In some embodiments, the first linker and the second linker interact indirectly, via interaction with one or mom intermediate compounds. For example, a first linker polynucleotide and a second linker polynucleotide may interact via complementarity with a different portion of an adapter polynucleotide. An adapter polynucleotide can comprise DNA, RNA, nucleotide analogues, non-canonical nucleotides, labeled nucleotides, modified nucleotides, or combinations thereof. Adapter polynucleotides can be single-stranded, double-stranded, or partial duplex. In general, a partial-duplex adapter comprises one or mom single-stranded regions and one or mom double-stranded regions. Double-stranded adapters can comprise two separate oligonucleotides hybridized to one another (also referred to as an “oligonucleotide duplex”), and hybridization may leave one or more 3⁺ overhangs, one or more 5′ overhangs, one or more bulges resulting from mismatched and/or unpaired nucleotides, or any combination of these. An adapter polynucleotide that interacts with both the first linker polynucleotide and the second linker polynucleotide may comprise a contiguous backbone. For example, the first and second linkers may hybridize to different portions of a single-stranded adapter polynucleotide. Alternatively, the first linker polynucleotide may hybridize to a first strand of a double-stranded linker, the second linker polynucleotide may hybridize to a second strand of a double-stranded linker, and the first and second strands of the adapter may hybridize with one another, such that the first and second linkers interact indirectly via sequence complementarity with the double-stranded adapter polynucleotide. An adapter polynucleotide may alternatively comprise a discontiguous backbone, such as when two or more double-stranded adapter polynucleotides (e.g., 2, 3, 4, 5, or more) hybridize in a chain, with the first linker polynucleotide hybridizing to one end of the chain and the second linker polynucleotide hybridizing to the other end of the chain (see U.S. Pat. No, 10,744,207).

In some embodiments, a subject complex comprises a targeting unit and a therapeutic unit, each of which comprises a linker, which interact to bring the two units together to form the complex. For example, a first moiety (such as a targeting moiety) that is conjugated to a first linker forms a complex with a second moiety (such as a therapeutic agent or a cell) that is conjugated to a second linker via an interaction between the first and second linkers. In some embodiments, the interaction between the linkers is the formation of a covalent bond. In some embodiments, the interaction between the linkers is a non-covalent interaction, such as electrostatic, hydrophobic, hydrogen bonding, Van Der Waals, or magnetic interactions. In some embodiments, the interaction between the linkers is a reversible interaction, such that the complex formed between the targeting unit and the therapeutic unit is a reversible complex. In general, a reversible complex is a complex that can be disrupted by changing one or more conditions to which the complex is subject, such as the conditions of a solution containing a reversible complex. For example, a reversible interaction may be disrupted by changing temperature (e.g., applying heat), changing pH (e.g., lowering or increasing pH), enzymatic activity (e.g., enzymatic degradation), changing ionic strength (e.g., decreasing salt concentration), or a combination of two or more of these. An example of a reversible interaction is the interaction between two complementary polynucleotides. For example, the first linker may be a polynucleotide with a single-stranded region that is complementary to a single-stranded region of the second linker polynucleotide, such that the linker hybridize to one another without further treatment but via the sequence complementarity. As another example, the first linker may be a polynucleotide with a double-stranded region that is complementary to a region of the second linker polynucleotide that is either double- or single-stranded, such that interaction between the first and second linkers is triggered by treating the combine moieties to render the complementary portion(s) of the linker(s) single-stranded (see U.S. Pat. No. 10,744,207).

A targeting unit embodied in a subject complex disclosed herein typically comprises a targeting moiety (such as an ingredient) which renders the targeting unit the ability to distinguish target from non-target by exhibiting preferential interaction or binding. Accordingly, a targeting moiety (such as an ingredient) of a targeting unit includes compounds or complexes having a higher binding affinity for a target compound or complex than for non-target compounds or complexes in a complex mixture. A targeting moiety may be selected based on having, or produced to have, a binding affinity for a desired target, such as a biomarker associated with a target cell (see U.S. Pat. No, 10,744,207).

Biological markers associated with a cell to which a targeting moiety such as an ingredient may be directed include cell surface markers. Non-limiting examples of cell surface markers include carbohydrates; glycolipids; glycoproteins; CD (cluster of differentiation) antigens present on cells of a hematopoietic lineage (e.g., CD2, CD4, CD8, CD21, etc.); γ-glutamyltranspeptidase; an adhesion protein (e.g., ICAM-1, ICAM-2, ELAM-1, VCAM-1); hormone, growth factor, cytokine, and other ligand receptors; ion channels; and the membrane-bound form of an immunoglobulin p chain. In some embodiments, the biological marker associated with a target cell is present on the surface of a target cells at about or less than about 100000, 50000, 10000, 5000, 1000, 750, 500, 100, 50, or fewer copies per cell. In some embodiments, the average density of a biological marker associated with the surface of a target cell in a population of target cells is about or less than about 100000, 50000, 10000, 5000, 1000, 750, 500, 100, 50, or fewer copies per cell. In some embodiments, the biological marker is associated with a target cell by way of increased concentration of the marker in a fluid surrounding the target cell or a tissue in which it resides than is found in fluid or tissue more distant from the target cell, such as where a cell secretes the biological marker. Of particular interest are biological markers associated with a disease or disease state; of particular further interest are disease-related biological markers expressed by a target cell (such as an abnormal cell) which is associated with the disease or the disease state. A vast variety of disease-related biological markers have been identified, and the corresponding targeting moieties have been generated, such as targeting moieties direct to alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a) antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdr1 gene product), multidrug-resistance-related antigen, p170, multidrug-resistance-related antigen, prostate specific antigen (PSA), CD56, and NCAM (see U.S. Pat. No. 10,744,207).

A linker suitable for conjugating to a therapeutic agent (such as cytotoxic cell) or a targeting moiety (e.g., an ingredient, an antibody) can be a member of a binding pair. A first member of a binding pair generally exhibits a higher affinity for a second member of the binding pair than for a non-member molecule. Examples of binding pairs include, but are not limited to, antigen-antibody, receptor-hormone, receptor-ligand, agonist-antagonist, lectin-carbohydrate, polynucleotide (RNA or DNA) hybridizing sequences, aptamer-target, avidin-biotin, streptavidin-biotin, leucine zipper-target polynucleotide, zinc finger-target polynucleotide, and the like. In some embodiments, the linker is an exogenous linker that is attached to a cell. In this context, “exogenous” is used to indicate that the linker was not produced by the cell to which it is conjugated. For example, an exogenous linker from a separate source may be mixed with a cell to conjugate the linker to the cell (see U.S. Pat. No, 10,744,207).

The linker and the moieties described herein can be conjugated by any suitable means known in the art. A linker conjugated to a targeting moiety (e.g., an ingredient, an antibody) or therapeutic unit (e.g., a cell) may be conjugated via a covalent or a non-covalent linkage. In some embodiments, the linker is conjugated to a native functional group of a targeting moiety (e.g., an ingredient, an antibody) or therapeutic unit, such as a functional group natively on a surface of a cell or a native group in a protein. The cell surface can include any suitable native functional group, such as amino acids and sugars. For example, reagents including maleimide, disulfide and the process of acylation can be used to form a direct covalent bond with a cysteine on a cell surface protein. Amide coupling can be used at an aspartate and glutamate to form an amide bond. Diazonium coupling, acylation, and alkylation can be used at a tyrosine on the cell surface to form an amide bond linkage. It is possible that any of the amino acids (20 amino acids or any unnatural amino acids) can be used to form the direct covalent bond that is the attachment of the oligonucleotide with the cell surface. The 20 amino acids are isoleucine, leucine, lysine, methionine, phenylalanine, threonine, tryptophan, and valine (essential amino acids), and alanine, asparagine, aspartate, cysteine, glutamate, glutamine, glycine, proline, serine, and tyrosine, the nonessential amino acids, and also arginine and histidine. In some embodiments, the native functional group can be an amino acid such as lysine, cysteine, tyrosine, threonine, serine, aspartic acid, glutamic acid or tryptophan. In other embodiments, the native functional group is lysine. In some other embodiments, the native functional group can be an N-terminal serine or threonine (see U.S. Pat. No, 10,744,207).

In some embodiments, the linker may be conjugated to the targeting unit or therapeutic unit using a coupling group. For example, the coupling group can be an activated ester (e.g., NHS ester, 1-ethyl-3-(3-dimethylaminopropyl) carbodiimide (EDC) ester, dicyclohexylcarbodiimide (DCC) ester, etc.), or an alkyl or acyl halide (e.g. —Cl, —Br, —I). In some embodiments, the activated ester is isolated and/or purified. In some embodiments, the activated ester is generated and/or used in situ. In some cases, the coupling group can directly conjugate to the therapeutic agent (e.g., surface of a cell used as a therapeutic agent) without pre-modification of the native functional group (e.g., amino acids). For example, the linker can be conjugated to the targeting unit or therapeutic unit by formation of a bond (e.g., an amide or ester bond) with an amino acid on a targeting moiety (e.g., antibody, aptamer) or a cell surface. In some embodiments, the coupling group is an NHS ester, which reacts with a nucleophilic native functional group on the targeting unit or therapeutic unit, resulting in an acylated product. For example, the native functional group can be an amine, which is conjugated via the NHS ester to form an amide. Alternatively, the native functional group can be a hydroxyl or a sulfhydryl group, which can be conjugated via the NHS ester to form an ester or a sulfhydryl ester linkage, respectively (see U.S. Pat. No, 10,744,207).

In some embodiments, the linker can be conjugated to the targeting unit or therapeutic unit using a bifunctional crosslinker. The bifunctional crosslinker can comprise two different reactive groups capable of coupling to two different functional targets such as peptides, proteins, macromolecules, semiconductor nanocrystals, or substrate. The two reactive groups can be the same or different and include but are not limited to such reactive groups as thiol, carboxylate, carbonyl, amine, hydroxyl, aldehyde, ketone, active hydrogen, ester, sulfhydryl or photoreactive moieties. In some embodiments, a cross-linker can have one amine-reactive group and a thiol-reactive group on the functional ends. In other embodiments, the bifunctional crosslinker can be an NHS-PEO-Maleimide, which comprise an N-hydroxysuccinimide (NHS) ester and a maleimide group that allow covalent conjugation of amine- and sulfhydryl-containing molecules. Further examples of heterobifunctional cross-linkers that may be used to conjugate the linker to the targeting unit or therapeutic unit include but are not limited to: amine-reactive+sulfhydryl-reactive crosslinkers, carbonyl-reactive+sulfhydryl-reactive crosslinkers, amine-reactive+photoreactive crosslinkers, sulfhydryl-reactive+photoreactive crosslinkers, carbonyl-reactive+photoreactive crosslinkers, carboxylate-reactive+photoreactive crosslinkers, and arginine-reactive+photoreactive crosslinkers (see U.S. Pat. No. 10,744,207).

Typical crosslinkers can be classified in the following categories (with exemplary functional groups):
1. Amine-reactive: the cross-linker couples to an amine (NH2) containing molecule, e.g., isothiocyanates, isocyanates, acyl azides, NHS esters, sulfonyl chlorides, aldehydes and glyoxals, epoxides and oxiranes, carbonates, arylating agents, imidoesters, carbodiimides, anhydrides, alkynes;
2. Thiol-reactive: the cross-linker couple to a sulfhydryl (SH) containing molecule, e.g., haloacetyl and alkyl halide derivates, maleimides, aziridines, acryloyl derivatives, arylating agents, thiol-disulfides exchange reagents;
3. Carboxylate-reactive: the cross-linker couple to a carboxylic acid (COOH) containing molecule, e.g., diazoalkanes and diazoacetyl compounds, such as carbonyldiimidazoles and carbodiimides;
4. Hydroxyl-reactive: the cross-linker couple to a hydroxyl (—OH) containing molecule, e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, isocyanates;
5. Aldehyde- and ketone-reactive: the cross-linker couple to an aldehyde (—CHO) or ketone (R2CO) containing molecule, e.g., hydrazine derivatives for schiff base formation or reduction amination;
6. Active hydrogen-reactive, e.g., diazonium derivatives for mannich condensation and iodination reactions; and
7. Photo-reactive, e.g., aryl azides and halogenated aryl azides, benzophenones, diazo compounds, diazirine derivatives (see U.S. Pat. No, 10,744,207).

For each category, i.e., whether a particular chemical targets a functional group, there are some subcategories, because some reactive groups are capable of reacting with several functional groups. For each of these subcategories there are many examples of chemicals. Many of these chemicals and the above list of subcategories can be found in, “Bioconjugate Techniques” by Greg T Hermanson, Academic Press, San Diego, 1996, which is hereby incorporated by reference (see U.S. Pat. No, 10,744,207).

In another embodiment, crosslinkers comprising polyethylene glycol (PEG), also referred to as polyethyleneoxide (PEO), spacers can be used as alternatives to reagents with purely hydrocarbon spacer arms. PEG spacers improve water solubility of reagent and conjugate, reduce the potential for aggregation of the conjugate, and increases flexibility of the crosslink, resulting in reduced immunogenic response to the spacer itself. By contrast to typical PEG reagents that contain heterogeneous mixtures of different PEG chain lengths, these PEO reagents are homogeneous compounds of defined molecular weight and spacer 5 arm length, providing greater precision in optimization and characterization of crosslinking applications. For example, succinimidyl-[(N-maleimidopropionamido)-hexaethyleneglycol] ester was used in the examples to make a stock solution by dissolving 5 mg of NHS-PEO6-maleimide (Pierce Biotechnology, Inc. Rockford, Ill, 61105) (see U.S. Pat. No, 10,744,207).

In some embodiments, the conjugation can result in a carboxyl or a carbonyl group, or amino or thio equivalents thereof. Examples of such groups include but are not limited to ketones, imides, thiones, amides, imidamides, thioamides, esters, imidoesters, thioesters, carbamates, ureas, thioureas, carbonates, carbonimidates and carbonthioates. In some embodiments, the conjugation can result in a hydrazone or an oxime bond. In some embodiments, the conjugation may result in a disulfide bond. In some embodiments, the linker can be conjugated using Native Chemical Ligation (NCL) methods. Additional examples of linkers and coupling groups are disclosed in WO2010118235A1, which is hereby incorporated by reference (see U.S. Pat. No, 10,744,207).

In some cases, the linker(s) can be directly conjugated to the cell surface. A cell is conjugated “directly” when the cell membrane (cell surface, outside of the cell, or component thereof) is not actively modified or changed before the attachment of the linker. Specifically, since the attachment is to a native functional group on the cell surface, “directly” means that the native functional group is not modified before the linker conjugation (see U.S. Pat. No, 10,744,207).

The buffer solution for the conjugation can be selected based on the choice of chemical linker or crosslinker and maintaining growth conditions for cells (i.e., to prevent cell lysis). In some embodiments, the buffer solution range is from pH 6-8 and does not contain the same functional groups used in the chemical linker to react with the conjugation linker (e.g., single-stranded polynucleotide). A pH of 7.2 can be used, but the pH does not have to be neutral, and typically is dependent on compatibility with the chemical reaction and the cellular conditions (see U.S. Pat. No, 10,744,207).

In some embodiments, the buffer solution is a phosphate buffer solution of neutral pH such that an N-hydroxsuccinimide (NHS) ester (e.g., NHS-PEO-maleimide) may be used as the coupling group. The reaction is generally carried out under conditions that allow the conjugation of the linker and the moiety (e.g., antibody, aptamer, cell surface). In some embodiments where an NHS ester crosslinker and phosphate buffer solution is used, the reactions am carried out at neutral pH (e.g., pH 7.2) and at room temperature for a specified period of time (e.g., about 1, 3, 5, 10, 15, 20, 30, 45, 60 or mom minutes) (see U.S. Pat. No, 10,744,207).

The linker can be a polynucleotide. Exemplary polynucleotides include, but are not limited to, deoxy-ribonucleic acid (DNA), ribonucleic acid (RNA), peptide nucleic acid (PNA), morpholino and locked nucleic acid (LNA), glycol nucleic acid (GNA), threose nucleic acid (TNA), single-stranded DNA (ssDNA), aptamer, and others nucleic acid moieties such as fluorinated nucleic acids. In some cases, the linker can be an aptamer. Aptamers am oligonucleotides that can adopt a three-dimensional structure and bind a specific target molecule (see U.S. Pat. No, 10,744,207).

In some embodiments, the disclosure provides a cell comprises at least two different targeting units complexed to its outer surface (e.g. a first type and a second type), wherein each of the different targeting units comprises a distinct ingredient that is not produced by the cell to which it is complexed, and interact specifically with a different binding site (such as different epitope) in a biological marker or with a different biological marker (e.g. a first type specifically interaction with (or binding to) a first epitope or first biological marker and a second type specifically interaction with (or binding to) a second epitope or a second biological marker). In some embodiments, the cell is complexed to mom than 2, 3, 4, 5, 6, 7, 8, 9, 10, 15, 20, or more different targeting units. In some embodiments, the ratio of one targeting unit to another targeting unit on the same cell surface is 1 to X, where X is about or mom than about 1, 1.5, 2, 3, 4, 5, 6, 7, 8, 9, 10, 15, 20, 25, 50, 75, 100, or mom (see U.S. Pat. No, 10,744,207).

In some embodiment, the disclosure provides a conjugated live cell comprising an ingredient that is not produced by the cell via the process of transcription and translation, but rather become associated with the cell by exogenous means (see U.S. Pat. No, 10,744,207).

### Methods

A wide variety of techniques are available in the steps of reacting an ingredient with a first polynucleotide to produce a targeting unit, and the step of reacting a live cell with a second polynucleotide to produce a conjugated cell. For example, the first polynucleotide can be conjugated to the ingredient using a bifunctional crosslinker. The bifunctional crosslinker can comprise two different reactive groups capable of coupling to two different functional targets such as peptides, proteins, macromolecules, semiconductor nanocrystals, or substrate. The two reactive groups can be the same or different and include but are not limited to such reactive groups as thiol, carboxylate, carbonyl, amine, hydroxyl, aldehyde, ketone, active hydrogen, ester, sulfhydryl or photoreactive moieties. In some embodiments, a cross-linker can have one amine-reactive group and a thiol-reactive group on the functional ends. In other embodiments, the bifunctional crosslinker can be an NHS-PEO-Maleimide, which comprise an N-hydroxysuccinimide (NHS) ester and a maleimide group that allow covalent conjugation of amine- and sulfhydryl-containing molecules (see U.S. Pat. No, 10,744,207).

In some embodiments, the biological marker is associated with a target cell by way of increased concentration of the marker in a fluid surrounding the target cell or a tissue in which it resides than is found in fluid more distant from the target cell, such as where a cell secretes or otherwise releases the biological marker (also referred to as extracellular markers). In some embodiments, the extracellular marker is a marker secreted or otherwise released from a cell in response to cell or tissue damage. For example, CK-MB and Troponin I am released 4 to 8 hours after the onset of chest pain, and am released after irreversible injury (i.e., necrosis) has occurred. Nourin-1 is an inflammatory polypeptide released within 5 minutes by heart tissues in response to myocardial ischemia (see U.S. Pat. No, 10,744,207).

In some embodiments, administering the complex (cytotoxic cells comprise a population of targeting units complexed to the surface of the cytotoxic cells) forms part of a therapy. The therapy may be for a neurological disease, disorder or deficit. The therapy may improve functional and/or cognitive recovery. The therapy may be of stroke, peripheral arterial disease, neuropathy or any other disease or disorder that requires tissue regeneration, revascularisation, or local anti-inflammatory action, including but not limited to, neurological disorder, disease or deficit, such as Parkinson's disease, Alzheimer's disease, stroke, or ALS; lysosomal storage disorders; cardiovascular disorders, such as myocardial infarction, congestive heart failure, peripheral arterial disease, diabetic ulcers, wound healing; diseases of the lung, including idiopathic pulmonary fibrosis, respiratory distress syndrome, chronic obstructive pulmonary disease, idiopathic pulmonary hypertension, cystic fibrosis and asthma; metabolic or inflammatory disorders, such as diabetes (i or ii), rheumatoid arthritis, osteoarthritis, lupus, crohn's disease, inflammatory bowel disease, or graft versus host disease; blindness-causing diseases of the retina, such as age-related macular degeneration, Stargardt disease, diabetic retinopathy, retinitis pigmentosa; and demyelinating diseases, such as multiple sclerosis, cerebral palsy, central pontine myelinolysis, tabes dorsalis, transverse myelitis, Devic's disease, progressive multifocal leukoencephalopathy, optic neuritis, leukodystrophies, Guillain-Barre syndrome, anti-MAG peripheral neuropathy and Charcot-Marie-tooth disease (see U.S. Pat. No, 10,744,207).

Complexes administered to a subject may be in any suitable form, such as a component of a pharmaceutical composition, which may additionally comprise one or more pharmaceutically acceptable carriers and optionally one or more additional therapeutic agents (such as in a combination composition). Pharmaceutically acceptable carriers include, but am not limited to, solvents, dispersion media, coatings, antibacterial and antifungal agents, isotonic and absorption delaying agents, and the like, compatible with pharmaceutical administration. See for example, Remington's Pharmaceutical Sciences (2005). Formulations suitable for parenteral administration, for example, include aqueous sterile injection solutions, which may contain antioxidants, buffers, bacteriostats, and solutes that render the formulation isotonic with the blood of the intended recipient; and aqueous and nonaqueous sterile suspensions that may include suspending agents and thickening agents. Supplementary active compounds can also be incorporated into the compositions. A pharmaceutical composition typically is formulated to be compatible with its intended route of administration. Examples of routes of administration include parenteral, e.g., intravenous, intradermal, subcutaneous, oral (e.g., inhalation), transdermal (topical), transmucosal, and rectal administrations. Solutions or suspensions used for parenteral, intradermal, or subcutaneous application can include one or more of the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic (see U.S. Pat. No, 10,744,207).

In some embodiments, a synergistically effective therapeutic amount of a complex produces a greater effect than the additive effects of the members of the complex when used alone (see U.S. Pat. No. 10,744,207).

### EXAMPLES

The following is a detailed description using the embodiments of the present invention as well as the techniques and features of the present invention, however, these embodiments are not intended to limit the invention, any changes and modifications made without departing from the spirit and scope of the invention by anyone who is familiar with this technology are intended to be included in the scope of the invention.

### Embodiment 1: Cytotoxic Function of the Ingredient-Complexed Cytotoxic Cells to Kill Ingredient-Responsive or Ingredient-Resistant Cancer Cells

The following describes a specific embodiment of preparing ingredient-complexed natural killer cells and ingredient-complexed gamma delta T cells, as well as the uses thereof, but the application of the invention is not limited thereto, which means any ingredient conjugated with any cytotoxic cells are intended to be included in the scope of the invention.

### Embodiment 1-1: Cytotoxic Function of the Ingredient-Complexed Human CD16⁺ Natural Killer Cells to Kill Ingredient-Responsive or Ingredient-Resistant Cancer Cells

### Embodiment 1-1-1 Culturing Human CD16⁺ Natural Killer Cells

Human CD16⁺ natural killer cell line that was deposited at NPMD on Sep. 2, 2019 with an address of #122, 2-5-8 Kazusakamatari, Kisarazu-shi, Chiba 292-0818, Japan, having the deposit number NITE BP-03017 (Referred to as “oNK cells”) was used in this embodiment. The human CD16⁺ natural killer cells that was deposited at NPMD having the deposit number NITE BP-03017 were centrifuged at a speed of 100˜1000×g for 3˜5 minutes. The supernatant was removed. After resuspending the cells with 1 mL of cell culture medium, the cell suspension was placed in a first container to make the first container contain 6.54×10⁵ human CD16⁺ natural killer cells in 40 mL cell culture medium;
wherein, the cell culture medium comprises:
(1) 0.5%-30%((Volume percent, vol %, v/v) Human platelet lysate;
(2) 100-3000 IU/mL Interleukin 2 (IL-2); and
(3) medium selected from a group consisting of DMEM culture medium (Dulbecco's Modified Eagle Medium), alpha modification of Eagle's minimum essential medium, and XVIVO 10 culture medium, or any combination thereof; and
wherein, the first container is, for example, T-75 flask, T-150 flask, T-225 flask, or G-Rex culture device; wherein, the G-Rex culture devices are culture containers equipped with air-permeable and water-impermeable membrane at their bottom to fully aerate culture.

After at least 7 days of culture, the cell suspension comprising cultured oNK cells (referred to as “cultured oNK cell suspension”) were obtained.

### Embodiment 1-1-2 Detection of Cell Condition and Cell Surface Markers of the Cultured Cells

There are two experimental trials in this embodiment. The first batch of the human CD16⁺ natural killer cells having the deposit number NITE BP-03017 and the second batch of the human CD16⁺ natural killer cells having the deposit number NITE BP-03017 were cultured respectively by the culture method of Embodiment 1-1-1 to obtain the cultured oNK cell suspensions of the first experimental trial and the cultured oNK cell suspensions of the second experimental trial. The first batch of the human CD16⁺ natural killer cells was cultured for 35 days in total, while the second batch of the human CD16⁺ natural killer cells was cultured for a long period of time until at least day 202.

Each sample of the cell suspensions, which were obtained at different time points in this Embodiment (Embodiment 1-1-2), was centrifuged; the supernatant was removed, the cells were resuspended in the buffer, then mix with 1 μL of propidium iodide (PI); 3 μL of the mixture of CD56 fluorescent labeled antibody (Cat. No, 318304, Biolegend, USA), CD3 fluorescent labeled antibody (Cat. No, 300410, Biolegend, USA), and CD2 fluorescent labeled antibody (Cat. No, 300222, Biolegend, USA); or 1 μL of CD16 fluorescent labeled antibody (Cat. No, 302016, Biolegend, USA). The cell sorter or flow cytometer was used to detect (1) whether the cells were stained with propidium iodide; (2) whether the cells exhibited CD56 molecules, CD3 molecules, and/or CD2 molecules; and (3) whether the cells exhibited CD16 receptor.

Please refer to Table 1 and Table 2. Table 1 shows the results of the cultured oNK cell suspensions obtained from the first experimental trial, and Table 2 shows the results of the cultured oNK cell suspensions obtained from the second experimental trial.

In Table 1, the first column “day” indicates the number of culture days; the second column “PI*” indicates the percentage of cells undergoing apoptosis or have died, based on the total number of the cells in the cultured oNK cell suspension as 100%; since natural killer cells, CD4⁺ T cells, and CD8⁺ T cells all exhibit CD56⁺ (Pernick, N, 2018), so the third column “CD56⁺” indicates the percentage of the total number of natural killer cells, CD4⁺ T cells, and CD8⁺ T cells, based on the total number of the cells in the cultured oNK cell suspension as 100%; since T cells all exhibit CD3⁺ (Pernick, N, 2018), the fourth column “CD3⁻” indicates the percentage of cells that are not T cells, based on the total number of the cells in the cultured oNK cell suspension as 100%; since natural killer cells, peripheral blood T cells, and most thymocytes all exhibit CD2⁺ (Pernick, N, 2018) and the cells to be detected in this Experiment are derived from peripheral blood, so the fifth column “CD2⁺” indicates the percentage of the total number of natural killer cells and T cells, based on the total number of the cells in the cultured oNK cell suspension as 100%; the sixth column “CD56⁺CD3⁻” indicates the percentage of natural killer cells, based on the total number of the cells in the cultured oNK cell suspension as 100%; the seventh column “CD56⁺CD2⁻” indicates percentage of the total number of natural killer cells and T cells, based on the total number of the cells in the cultured oNK cell suspension as 100%; since natural killer cell and macrophage exhibit CD16⁺ (Pernick, N, 2018), and CD16 is involved in Antibody-dependent cell cytotoxicity (ADCC), the eighth column “CD16⁺” indicates the percentage of the total number of natural killer cells and macrophoges with ADCC function, based on the total number of the cells in the cultured oNK cell suspension as 100%; the ninth column “CD56⁺CD16⁻” indicates the percentage of natural killer cells with ADCC function, based on the total number of natural killer cells in the cultured oNK cell suspension (i.e., CD56⁺CD3⁻ cells) as 100.

The indication of the first to eighth columns in Table 2 is the same as in Table 1; when the ninth column “killing test” marks “✓” symbol, this indicates that the cytotoxic function of the cells in the cultured oNK cell suspension at certain time point was simultaneously tested and confirmed that the cells have cytotoxic function.

Table 1 the test result of cell condition and cell surface marker of the cells in the cultured oNK cell suspension obtained by culturing the first batch of the purified CD16⁺ cell population.

| | PI⁺ | CD56⁺ | CD3⁻ | CD2⁺ | CD56⁺CD3⁻ | CD56⁺CD2⁺ | CD16⁺ | CD56⁺/CD16⁺ |
|---|---|---|---|---|---|---|---|---|
| | (% of | (% of | (% of | (% of | (% of | (% of | (% of | (% of |
| | total | total | total | total | total | total | total | CD56⁺CD3⁻ |
| Day | cells) | cells) | cells) | cells) | cells) | cells) | cells) | cells) |
| 7 | 6.54 | 99.45 | 100 | 98.08 | 98.76 | 99.01 | 90.17 | — |
| 16 | 5.65 | 99.08 | 99.96 | 98.86 | 98.21 | 98.78 | 90.35 | — |
| 21 | 7.34 | 99.56 | 99.9 | 98.75 | 98.71 | 99.33 | 90.67 | — |
| 23 | — | — | — | — | — | — | — | 88.79 |
| 26 | — | — | — | — | — | — | — | 90.51 |
| 28 | 7.18 | 99.33 | 99.88 | 99.22 | 98.29 | 99.15 | 92.36 | — |
| 30 | — | — | — | — | — | — | — | 92.11 |
| 35 | — | — | — | — | — | — | — | 91.37 |

Table 2 the test results of cell condition, cell surface marker and cytotoxicity of the cells in the cultured oNK cell suspension obtained by culturing the second batch of the purified CD16⁺ cell population.

| | | PI⁺ | CD56⁺ | CD3⁻ | CD2⁺ | CD56⁺CD3⁻ | CD56⁺CD2⁺ | CD16⁺ | |
|---|---|---|---|---|---|---|---|---|---|
| | Total | (% of | (% of | (% of | (% of | (% of | (% of | (% of | |
| | cell | total | total | total | total | total | total | total | Killing |
| Day | number | cells) | cells) | cells) | cells) | cells) | cells) | cells) | test |
| 7 | 1.61 × 10⁶ | 5.97 | 99.45 | 100.00 | 98.08 | 98.76 | 99.01 | 90.17 | |
| 16 | 1.01 × 10⁹ | 5.65 | 99.09 | 99.96 | 98.86 | 98.21 | 98.8 | 90.36 | |
| 21 | 2.53 × 10⁹ | 6.2 | 99.56 | 99.91 | 98.75 | 98.72 | 99.33 | 90.7 | |
| 28 | 5.06 × 10⁹ | 6.46 | 99.33 | 99.88 | 99.22 | 98.29 | 99.15 | 92.36 | |
| 37 | 1.01 × 10¹⁰ | 10.5 | 98.85 | 99.99 | 98.48 | 97.65 | 98.66 | 91.96 | |
| 42 | 1.62 × 10¹⁰ | 9.63 | 99.15 | 100.00 | 98.24 | 98.06 | 98.8 | 93.09 | |
| 49 | 3.24 × 10¹⁰ | 6.31 | 98.99 | 100.00 | 94.5 | 97.71 | 97.83 | 94.04 | |
| 65 | 1.13 × 10¹¹ | 4.41 | 99.15 | 99.99 | 98.55 | 97.81 | 98.85 | 90.35 | |
| 92 | 1.81 × 10¹⁵ | 2.7 | 99.62 | 99.99 | 99.43 | 98.58 | 99.42 | 85.99 | ✓ |
| 97 | 3.25 × 10¹⁶ | 7.91 | 99.23 | 99.90 | 99.58 | 98.3 | 99.05 | 86.98 | ✓ |
| 103 | 6.50 × 10¹⁷ | 3.17 | 99.65 | 99.82 | 99.5 | 98.71 | 99.45 | 83.88 | ✓ |
| 134 | 1.35 × 10²² | 3.09 | 99.62 | 99.99 | 99.68 | 98.75 | 99.42 | 86.18 | ✓ |
| 166 | 3.24 × 10²⁷ | 4.74 | 99.17 | 100.00 | 99.06 | 99.05 | 99.61 | 89.93 | ✓ |
| 184 | 1.30 × 10³³ | 7.87 | 99.61 | 99.99 | 98.23 | 98.77 | 99.37 | 92.38 | ✓ |
| 202 | 1.04 × 10³⁹ | 5.36 | 99.59 | 99.96 | 97.52 | 98.94 | 99.33 | 93.02 | ✓ |

### Embodiment 1-1-3 Detection of Activation Markers, Inhibitory Markers, and Other NK Cell Markers of the Cultured Cells

Cell suspensions obtained by culturing for 93 days with the culture method disclosed in the Embodiment 1-1-1 (refer to as 93-day cultured oNK cell suspension) were used in this embodiment. Cells in the 93-day cultured oNK cell suspension were evenly assigned into 19 groups. Cells in the first group were centrifuged: the supernatant was removed, the cells were resuspended in the buffer, then mixed with 1 μL of CD56 fluorescent labeled antibody (Cat. No, 318304. Biolegend. USA), 1 μL of CD3 fluorescent labeled antibody (Cat. No, 300410. Biolegend. USA), and 1 μL of CD2 fluorescent labeled antibody (Cat. No. 300222. Biolegend. USA) to simultaneously label cells expressing CD56 molecule. CD3 molecule, and/or CD2 molecule. Finally, the cell sorter or flow cytometer was used to analyze whether the cells exhibited CD56 molecules. CD3 molecules, and/or CD2 molecules, and the percentage of cells with various cell surface makers was calculated.

Cells in the other 18 groups were centrifuged; the supernatant was removed, the cells were resuspended in the buffer, then respectively mixed with 1 μL of CD16 fluorescent labeled antibody (Cat. No. 302016, Biolegend. USA). CD45 fluorescent labeled antibody (Cat. No, 368512. Biolegend. USA), CD4 fluorescent labeled antibody (Cat. No, 300514. Biolegend, USA), CD8 fluorescent labeled antibody (Cat. No, 344706. Biolegend. USA), CD19 fluorescent labeled antibody (Cat. No, 302210. Biolegend. USA). CD25 fluorescent labeled antibody (Cat. No, 302614, Biolegend. USA). NKp30 fluorescent labeled antibody (Cat. No, 325214. Biolegend. USA). NKG2D fluorescent labeled antibody (Cat. No, 320812. Biolegend. USA). NKp44 fluorescent labeled antibody (Cat. No, 325116, Biolegend, USA). NKp46 fluorescent labeled antibody (Cat. No, 331916, Biolegend. USA), CD27 fluorescent labeled antibody (Cat. No, 47-0279-42. Invitrogen. USA). OX40 fluorescent labeled antibody (Cat. No, 350004. Biolegend. USA), CD107a fluorescent labeled antibody (Cat. No, 328630. Biolegend, USA), NKG2A fluorescent labeled antibody (Cat. No. FAB1059P. R&D Systems. USA), PD-1 fluorescent labeled antibody (Cat. No, 367406, Biolegend, USA). TIGIT fluorescent labeled antibody (Cat. No, 372704. Biolegend. USA). SIRPα fluorescent labeled antibody (Cat. No, 372104, Biolegend, USA), and CD158 fluorescent labeled antibody (Cat. No. FAB1848P. R&D Systems. USA).

Finally, the cell sorter was used to analyze whether the cells exhibited CD16 receptor, CD45 marker, CD4 marker, CD8 marker, CD19 marker, CD25 marker, NKp30 marker. NKG2D marker, NKp44 marker. NKp46 marker, CD27 marker, OX40 marker, CD107a marker, NKG2A marker, PD-1 marker, TIGIT marker, SIRPα marker, and CD158 marker.

Among these markers. CD16, CD25, NKp30, NKG2D, NKp44, NKp46, and CD107a are activation markers, whereas NKG2A, PD-1, TIGIT, SIRPα, CD27, OX40, and CD158 are inhibitory markers. Based on the knowledge of those skilled in the art, expression of activation markers potentiates anti-tumor activity of NK cells, whereas expression of inhibitory markers potentiates function inhibition of NK cells.

Please refer to Table 3. Table 3 shows the test result of the activation markers, inhibitory markers, and other NK cell markers of the cells in cell suspensions obtained from Embodiments 1-1-1.

Table 3 shows that the purified CD16⁺ populations express CD56 (98.0±0.2%), CD2 (99.5±0.2%), CD45 (99.7±0.1%), CD4 (0.8±0.3%). CD3 (0.0±0.0%), CD8 (0.0±0.0%), CD19 (0.0±0.0%). CD16 (85.7±7.0%). CD25 (42.3±13.1%), NKp30 (93.6±4.3%). NKG2D (46.1±17.4%). NKp44 (75.1±13.3%). NKp46 (46.4±16.9%), CD27 (0.62±0.08%). OX40 (0.11±0.03%). CD107a (96.1±4.3%). NKG2A (0.14±0.15%), PD-1 (27.0±19.4%), TIGIT (4.3±6.5%), SIRPα (3.2±3.0%), and CD158 (0.4±0.3%). All of the aforesaid cells expressing CD16 receptor in the purified CD16⁺ cell population have the feature of CD3-CD56⁺ after analysis. Preferably, the aforesaid cells expressing CD16 receptor in the purified CD16⁺ cell population is positive for CD2. CD45, and CD4 and negative for CD8 and CD19. The positiveness of CD4 is an unexpected result.

Table 3 the test result of the activation markers, inhibitory markers, and other NK cell markers of the cells in the cultured oNK cell suspension.

| Marker | CD56 | CD2 | CD45 | CD4 | CD3 | CD8 | CD19 |
|---|---|---|---|---|---|---|---|
| % of positive | 98.0 ± 0.2 | 99.5 ± 0.2 | 99.7 ± 0.1 | 0.8 ± 0.3 | 0.0 ± 0.0 | 0.0 ± 0.0 | 0.0 ± 0.0 |
| population | | | | | | | |

| Marker | CD16 | CD25 | NKp30 | NKG2D | NKp44 | NKp46 | CD27 | OX40 | CD107a |
|---|---|---|---|---|---|---|---|---|---|
| % of positive | 85.7 ± 7.0 | 42.3 ± 13.1 | 93.6 ± 4.3 | 46.1 ± 17.4 | 75.1 ± 13.3 | 46.4 ± 16.9 | 0.62 ± 0.08 | 0.11 ± 0.03 | 96.1 ± 4.3 |
| population | | | | | | | | | |

| Marker | NKG2A | PD-1 | TIGIT | SIRPα | CD158 |
|---|---|---|---|---|---|
| % of positive | 0.14 ± 0.15 | 27.0 ± 19.4 | 4.3 ± 6.5 | 3.2 ± 3.0 | 0.4 ± 0.3 |
| population | | | | | |

### Embodiment 1-1-4 Prepare Trastuzumab-Complexed Human CD16⁺ Natural Killer Cells and Cetuximab-Complexed Human CD16⁺ Natural Killer Cells

The cultured oNK cell suspensions that was obtained by culturing for 16 days with the culture method disclosed in the Embodiment 1-1-1 (16-day cultured oNK cell suspension of the present invention, refer to as 16-day cultured oNK cell suspension) were used to prepare ingredient-complexed human CD16⁺ natural killer cells. After binding ingredient such as trastuzumab or cetuximab to cells in the 16-day cultured oNK cell suspension using a cell linker and an ingredient linker which are complementary, the trastuzumab-complexed human CD16⁺ natural killer cell suspension or cetuximab-complexed human CD16⁺ natural killer cells suspension was obtained.

The procedure of binding ingredient (e.g., trastuzumab, cetuximab, rituximab, alemtuzumab, avelumab, daratumumab, elotuzumab, codrituzumab) to cells (e.g., all of the cells in the cultured oNK cell suspension in this experiment, human CD16⁺ natural killer cells that was deposited at NPMD having the deposit number NITE BP-03017, other natural killer cells, gamma delta T cells, other T lymphocytes, macrophages, or other cytotoxic cells) was as follows: (A) The step of preparing cell linker and binding the cell linker to the cell in order to prepare a cell-ssDNA conjugate: (B) The step of preparing ingredient linker and binding the ingredient linker to ingredient in order to prepare the ingredient-ssDNA conjugate: (C) Mixing cell-ssDNA conjugate and ingredient-ssDNA conjugate to combine cell-ssDNA conjugate and ingredient-ssDNA conjugate through the cell linker and its complementary sequence on the ingredient linker in order to prepare ingredient-complexed cells such as ingredient-complexed human CD16⁺ natural killer cells or ingredient-complexed human gamma delta T cells.

Wherein the step (A) of preparing cell linker and binding the cell linker to the cell comprises the following steps (a1)˜(a4):
Step (a1) A first single strand DNA was obtained, wherein the sequence of the first single strand DNA was SEQ ID NO: 2. SEQ ID NO: 3, or SEQ ID NO: 7.
Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).
Step (a3) 10˜500 μL cell linker stock and 0.1˜10 μL NHS-Maleimide (commercially available from Fisher Scientific) were mixed and incubated for 1˜60 minute(s).
Step (a4) The mixture obtained from Step (a3) were mixed with 1×10⁶˜1×10⁸ cells (such as all of the cells including cultured oNK cells in the cultured oNK suspension) and incubated for 1-60 minutes to obtain cell-ssDNA conjugate.

The step (B) of preparing ingredient linker and binding the ingredient linker to ingredient comprises the following steps (b1)˜(b4):
Step (b1) A second single strand DNA was obtained, wherein the sequence of the second single strand DNA was SEQ ID NO: 5. SEQ ID NO: 6, or SEQ ID NO: 8, and the sequence of the second single strand DNA is the complementary strand to the first single strand DNA.
Step (b2) The 5′ end of the second single strand DNA was modified as 5′ end thiol-modified second single strand DNA to obtain an ingredient linker stock. The ingredient linker stock is also commercially available from Integrated DNA Technologies. Actual methods of modification are known, or will be apparent, to those skilled in the art (Zimmermann, J, 2010).
Step (b3) 10˜500 μL ingredient linker stock and 0.1˜10 μL NHS-Maleimide (commercially available from Fisher Scientific) were mixed and incubated for 1˜60 minute(s).
Step (b4) The mixture obtained from Step (b3) were mixed with the ingredients (such as 10˜100 μL ingredient stock that is commercially available from Roche) and incubated for 10 minutes to 3 hours to obtain ingredient-ssDNA conjugate.

The cell-ssDNA conjugate and the ingredient-ssDNA conjugate were mixed to obtain ingredient-complexed cells such as cells in the trastuzumab-complexed human CD16⁺ natural killer cell suspension or cells in the cetuximab-complexed human CD16⁺ natural killer cell suspension.

### Embodiment 1-1-5 Cytotoxic Function of the Trastuzumab-Complexed Human CD16⁺ Natural Killer Cells to Kill Trastuzumab-Responsive or Trastuzumab-Resistant Human Breast Cancer Cells

xCELLigence Real Time Cell Analysis System (xCELLigence RTCA system, ACEA Biosciences Inc., USA) was used in this embodiment to detect the cytotoxic ability of the effector cell toward target cells. This embodiment comprised a 96 well xCELLigence E-Plate to carry out cytotoxicity test, and the wells in xCELLigence E-Plate were divided into control wells. ACE1702 ET2 experimental well. ACE1702 ET5 experimental well. Ctrl-oNK ET2 experimental well, Ctrl-oNK ET5 experimental well, and target cell maximum lysis control well.

The effector cells used in this embodiment were the cells in the 37-day cultured oNK cell suspension and the cells in the trastuzumab-complexed human CD16⁺ natural killer cell suspension, wherein the trastuzumab is an antibody against HER2 protein with product name as Herceptin (purchased from Roche, Swiss). The target cells were human breast cancer cell line BT474 (HTB-20, purchased from ATCC), or BT474's derived trastuzumab-resistant clone BT474 Clone 5 (CRL-3247, purchased from ATCC). Target cells (BT474 or BT474 Clone 5) were seeded in control well, ACE1702 ET2 experimental well, ACE1702 ET5 experimental well. Ctrl-oNK ET2 experimental well. Ctrl-oNK ET5 experimental well, and target cell maximum lysis control well, so that each well-contained 20000 target cells, and then allowed it to sit 30 minutes.

A sample of the trastuzumab-complexed human CD16⁺ natural killer cell suspension (ACE1702) was added to the ACE1702 ET2 experimental well and ACE1702 ET5 experimental well, and the ratio of the number of effector cell to the number of target cell (BT474 or BT474 Clone 5) was 2 and 5 respectively. A sample of the 37-day cultured oNK cell suspension (Ctrl-oNK) was added to the Ctrl-oNK ET2 experimental well and Ctrl-oNK ET5 experimental well, and the ratio of the number of effector cell to the number of target cell (BT474 or BT474 Clone 5) was 2 and 5 respectively as well. Added a tenth equal volume of lysis buffer to the sample into target cell maximum lysis control well; any sample or lysis buffer was not added to control well. The xCELLigence E-Plate was placed in the xCELLigence Real Time Cell Analysis System to detect real time change in the cell index (CI) under the condition of 37° C. and 5% carbon dioxide.

Wherein, the greater the number of target cells attached to the bottom of the xCELLigence E-Plate, the higher the cell index detected by the xCELLigence Real Time Cell Analysis System. Therefore, the cell index can be used to convert the percentage of target cells that are lysed in the experimental well. The formula used to convert the cell index to the percentage of target cells that are lysed in the experimental well is:

$$Percentage of lysed target cell ( \% ) = \text{} 1 - [ ( cell index of experimental well - cell index of target cell maximum lysis control well ) \div ( cell index of control well - cell index of target cell maximum lysis control well ) ] \times 100 \%$$

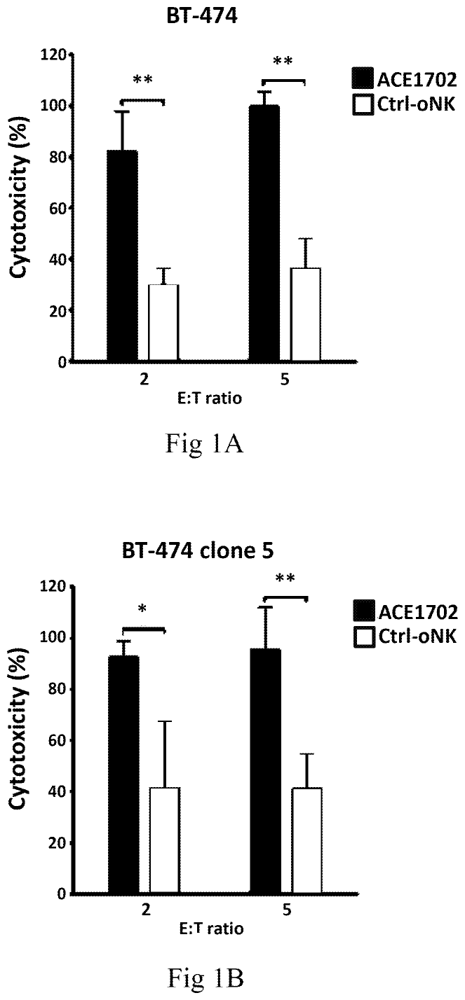

Please refer to FIGS. 1A and 1B. FIG. 1A is bar chart presenting the comparison of the cytotoxic function between the trastuzumab-complexed human CD16⁺ natural killer cells and the non-complexed human CD16⁺ natural killer cells to kill trastuzumab-responsive cancer cells (trastuzumab-responsive human breast cancer cell line BT474). FIG. 1B is a bar chart presenting the comparison of the cytotoxic function between the trastuzumab-complexed human CD16⁺ natural killer cells and the non-complexed human CD16⁺ natural killer cells to kill trastuzumab-resistant cancer cells (trastuzumab-resistant human breast cancer cell line BT474 Clone 5). As shown in FIGS. 1A and 1B, the ability of human CD16⁺ natural killer cells (Ctrl-oNK) to kill trastuzumab-responsive human breast cancer cell line BT474 and trastuzumab-resistant human breast cancer cell line BT474 Clone 5 were similar. Unexpectedly, the killing capacity of trastuzumab-complexed human CD16⁺ natural killer cells (ACE1702, which is human CD16⁺ natural killer cell complexed with trastuzumab) to trastuzumab-responsive human breast cancer cell line BT474 and trastuzumab-resistant human breast cancer cell line BT474 Clone 5 were also similar.

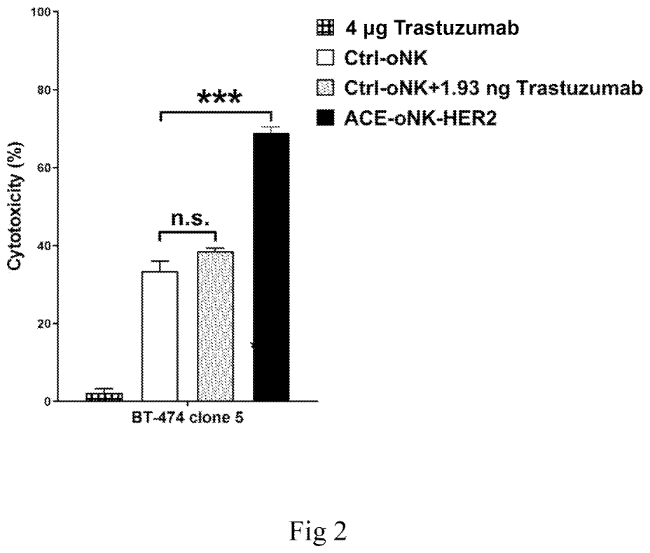

Please further refer to FIG. 2. FIG. 2 is a bar chart presenting the cytotoxic effect of trastuzumab, human CD16⁺ natural killer cells, human CD16⁺ natural killer cells co-cultured with trastuzumab, and trastuzumab-complexed human CD16⁺ natural killer cells on trastuzumab-resistant cancer cells. As shown in FIG. 2, high-dose trastuzumab (4 μg) can only kill a small amount of trastuzumab-resistant cancer cells, and the killing effect of human CD16⁺ natural killer cells co-cultured with 1.93 ng trastuzumab (Ctrl-oNK+1.93 ng trastuzumab) is not significantly higher than the effect of human CD16⁺ natural killer cells (Ctrl-oNK). Unexpectedly, the killing effect of human CD16⁺ natural killer cells complexed with 1.93 ng trastuzumab (ACE-oNK-HER2, which is human CD16⁺ natural killer cell complexed with 1.93 ng trastuzumab) is significantly higher than the killing effect of human CD16⁺ natural killer cells (Ctrl-oNK) and causes about 2-folds increase of cytotoxicity. This is an unexpected result and indicates that trastuzumab and human CD16⁺ natural killer cells (Ctrl-oNK) in the trastuzumab-complexed human CD16⁺ natural killer cells manifest synergy in killing trastuzumab-resistant cancer cells.

Therefore, ingredient-complexed cytotoxic cells such as trastuzumab-complexed human CD16⁺ natural killer cells (ACE1702) obtained by complexing trastuzumab and human CD16⁺ natural killer cells (Ctrl-oNK) can be used for treating abnormal cells that are resistant, refractory, insensitive, non-responsive, or inadequately responsive to the ingredient: so that ingredient-complexed cytotoxic cells such as trastuzumab-complexed human CD16⁺ natural killer cells (ACE1702) is able to solve the problem that no drug can cure abnormal cells resistant, refractory, insensitive, non-responsive, or inadequately responsive to current FDA-approved drugs (such as FDA-approved trastuzumab), as well as improving the treatment effectiveness of an ingredient in a subject who is resistant, refractory, insensitive, non-responsive, or inadequately responsive to the ingredient.

### Embodiment 1-1-6 Cytotoxic Function of the Cetuximab-Complexed Human CD16⁺ Natural Killer Cells to Kill Cetuximab-Responsive or Cetuximab-Resistant Human Colon Cancer Cells

The experimental method of this embodiment is almost the same as that of Embodiment 1-1-5, except that:
(1) the effector cell used in this Experiment were \textcircled{1} the cells in the 16-day cultured oNK cell suspension, or \textcircled{2} the cells in the cetuximab-complexed human CD16⁺ natural killer cell suspension, wherein the cetuximab is an antibody against epidermal growth factor receptor (EGFR) with product name as Erbitux (purchased from Merck, Bristol-Myers-Squibb); and
(2) the target cells used in this Experiment were \textcircled{1} human colorectal adenocarcinoma cell line HCC827-luc (JCRB1516, purchased from JCRB) that is not resistant to cetuximab, or (2) cetuximab-resistant human colorectal adenocarcinoma cell line HT-29 (HTB-38, purchased from ATCC).

Inventors of the present invention expect that:
(1) The ability of human CD16⁺ natural killer cells (Ctrl-oNK) to kill cetuximab-responsive human lung adenocarcinoma cell line HCC827-luc and cetuximab-resistant human colorectal adenocarcinoma cell line HT-29 were similar. Unexpectedly, the killing capacity of cetuximab-complexed human CD16⁺ natural killer cells to cetuximab-responsive human lung adenocarcinoma cell line HCC827-luc and cetuximab-resistant human colorectal adenocarcinoma cell line HT-29 were also similar.
(2) cetuximab can only kill a small amount of cetuximab-resistant cancer cells, and the killing effect of human CD16⁺ natural killer cells co-cultured with cetuximab (Ctrl-oNK+cetuximab) is not significantly higher than the effect of human CD16⁺ natural killer cells (Ctrl-oNK). Unexpectedly, the killing effect of human CD16⁺ natural killer cells complexed with cetuximab is significantly higher than the killing effect of human CD16⁺ natural killer cells (Ctrl-oNK). This is an unexpected result and indicates that cetuximab and human CD16⁺ natural killer cells (Ctrl-oNK) in the cetuximab-complexed human CD16⁺ natural killer cells manifest synergy in killing cetuximab-resistant cancer cells.

Therefore, ingredient-complexed cytotoxic cells such as cetuximab-complexed human CD16⁺ natural killer cells obtained by complexing cetuximab and human CD16⁺ natural killer cells (Ctrl-oNK) can be used for treating abnormal cells that are resistant, refractory, insensitive, non-responsive, or inadequately responsive to the ingredient, so that ingredient-complexed cytotoxic cells such as cetuximab-complexed human CD16⁺ natural killer cells is able to solve the problem that no drug can cure abnormal cells that are resistant, refractory, insensitive, non-responsive, or inadequately responsive to current FDA-approved drugs (such as FDA-approved cetuximab), as well as improving the treatment effectiveness of an ingredient in a subject who is resistant, refractory, insensitive, non-responsive, or inadequately responsive to the ingredient.

### Embodiment 1-1-7 Cytotoxic Function of the Cetuximab-Complexed Human CD16⁺ Natural Killer Cells to Kill Cetuximab-Responsive or Cetuximab-Resistant Human Tongue Cancer Cells

The experimental method of this embodiment is almost the same as that of Embodiment 1-1-5, except that:
(1) the effector cell used in this Experiment were \textcircled{1} the cells in the 56-day cultured oNK cell suspension, or \textcircled{2} the cells in the cetuximab-complexed human CD16⁺ natural killer cell suspension; and
(2) the target cells used in this Experiment were \textcircled{1} human tongue squamous carcinoma cell line HSC-4 (JCRB0264, purchased from JCRB) that is not resistant to cetuximab, or \textcircled{2} cetuximab-resistant human tongue squamous carcinoma cell line SAS (JCRB0260, purchased from JCRB).

Inventors of the present invention expect that:
(1) The ability of human CD16⁺ natural killer cells (Ctrl-oNK) to kill cetuximab-responsive human tongue squamous carcinoma cell line HSC-4 and cetuximab-resistant human tongue squamous carcinoma cell line SAS were similar. Unexpectedly, the killing capacity of cetuximab-complexed human CD16⁺ natural killer cells to cetuximab-responsive human tongue squamous carcinoma cell line HSC-4 and cetuximab-resistant human tongue squamous carcinoma cell line SAS were also similar;
(2) cetuximab can only kill a small amount of cetuximab-resistant cancer cells, and the killing effect of human CD16⁺ natural killer cells co-cultured with cetuximab (Ctrl-oNK+cetuximab) is not significantly higher than the effect of human CD16⁺ natural killer cells (Ctrl-oNK). Unexpectedly, the killing effect of human CD16⁺ natural killer cells complexed with cetuximab is significantly higher than the killing effect of human CD16⁺ natural killer cells (Ctrl-oNK). This is an unexpected result and indicates that cetuximab and human CD16⁺ natural killer cells (Ctrl-oNK) in the cetuximab-complexed human CD16⁺ natural killer cells manifest synergy in killing cetuximab-resistant cancer cells.

Therefore, ingredient-complexed cytotoxic cells such as cetuximab-complexed human CD16⁺ natural killer cells obtained by complexing cetuximab and human CD16⁺ natural killer cells (Ctrl-oNK) can be used for treating abnormal cells that are resistant, refractory, insensitive, non-responsive, or inadequately responsive to the ingredient to solve the problem that no drug can cure abnormal cells that are resistant, refractory, insensitive, non-responsive, or inadequately responsive to current FDA-approved drugs (such as FDA-approved cetuximab), as well as improving the treatment effectiveness of an ingredient in a subject who is resistant, refractory, insensitive, non-responsive, or inadequately responsive to the ingredient.

### Embodiment 1-2: Cytotoxic Function of the Ingredient-Complexed Human Gamma Delta T Cells to Kill Ingredient-Responsive or Ingredient-Resistant Cancer Cells

### Embodiment 1-2-1 Culturing Human Gamma Delta T Cells

The peripheral blood mononuclear cells (PBMC) were obtained from an autologous subject or an allogeneic subject, wherein the autologous or allogeneic subject was a healthy subject or a cancer patient. Preferably, the PBMC were obtained from a healthy donor.

5 vials of PBMC (vial number: #1217-01, #1217-02, #1217L-01, 1217L-02, and #1231, obtained from different donors) were placed in a 37° C. water bath to thaw the cells respectively.

In minimum preparation, 1 mL of the thawed peripheral blood mononuclear cells (PBMC) in each vial was mixed with 9 mL of complete growth medium to suspend cells respectively; wherein the complete growth medium was RPMI-1640 based (commercially available from Gibco. Sigma Aldrich. Biological Industries. STEMCELL Technologies, etc.) and supplemented with 1% (v/v)˜30% (v/v) human platelet lysates (commercially available from StemCell Technologies. Sigma Aldrich, Millipore, etc.) with 100˜2500 IU/mL (0.0612˜1.53 μg/mL) human interleukin 2 (IL-2). The cell suspension was centrifuged at 400×g at room temperature for 3-5 minutes followed by replacement of supernatant with 5 mL fresh complete growth medium. The cell suspension containing 5×10⁶˜200×10⁶ cells was transferred into a culture container (for example, T-75 flask, T-150 flask. T-225 flask, G-Rex culture device, etc.), and the medium volume was filled up to the max capacity of the culture container. The G-Rex culture devices are culture containers equipped with air-permeable and water-impermeable membrane at their bottom to fully aerate culture. The culture medium was supplemented with 1%˜30% human platelet lysates and spiked with 0.1˜20 μM of Zoledronate with 200-3000 IU/mL of human IL-2 on Day 0. On Day 2 and Day 4, cell culture was replenished with 100˜2500 IU/mL of human IL-2. On Day 7, the cell culture was replaced with fresh complete growth medium supplemented with 1%˜30% human platelet lysates and contained 100˜2500 IU/mL of human IL-2. On Day 9 and Day 11, 100˜2500 IU/mL of human IL-2 was replenished in the cell culture. On day 10 and day 14, the cell suspensions comprising gamma delta T cells (referred to as “gamma delta T cell suspension”) in each batch of the cultured cells were collected.

Cell culture was replaced with 100-2500 IU/mL of IL-2 and 1%-30% human platelet lysates-containing fresh complete growth medium for extended expansion. Similar expansion procedure can be performed in larger culture of freshly isolated PBMC.

### Embodiment 1-2-2 Detection of Cell Condition and Cell Surface Markers of the Cultured Cells

Cells obtained from Embodiment 1-2-1 were centrifuged at room temperature at 400×g for 3 minutes. Supernatant was removed and cell pellet was resuspended and washed with 1 mL of phosphate buffer saline (DPBS). Cell suspension was then centrifuged one more time, and the supernatant was removed. Cell pellet was resuspended with Dulbecco's Phosphate-Buffered Saline (DPBS), and 0.1 mL of cell suspension (5×10⁵ cells) was aliquoted to 1.5 mL eppendorf. One microliter of fluorescent dye FITC-conjugated anti-TCRV82 (BioLegend, #331406) and PE-Cy5-conjugated anti-CD3 (BioLegend, #300410) antibodies were mixed with one aliquoted cell suspension. The anti-TCR82 and anti- CD3 antibodies-stained cells were individually stained with PE-conjugated mouse anti-human CD56 (BioLegend, #304606), mouse anti-human CD16 (BioLegend, #331406), mouse anti-human NKG2D (BioLegend, #320812), mouse anti-human NKp44 (BioLegend, #325116), mouse anti-human NKp46 (BioLegend, #331916), APC-Cy7-conjugated anti-CD107a (BioLegend, #328630) and PE-conjugated anti-CD69 (BioLegend, #310906) antibodies at room temperature for 10 minutes avoid of light. One more aliquoted cell suspension was stained with Alexa Fluor® 488-conjugated TCRαβ (BioLegend, #306712) and PE-Cy5-conjugated CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended viable TCRVδ2⁺/CD3⁺ gated cells was analyzed for percentage and/or mean fluorescence intensity of CD56⁺, CD16⁺, NKG2D⁺, NKp44⁺, NKp46⁺, CD107a⁺ and CD69⁺ populations by flow cytometry.

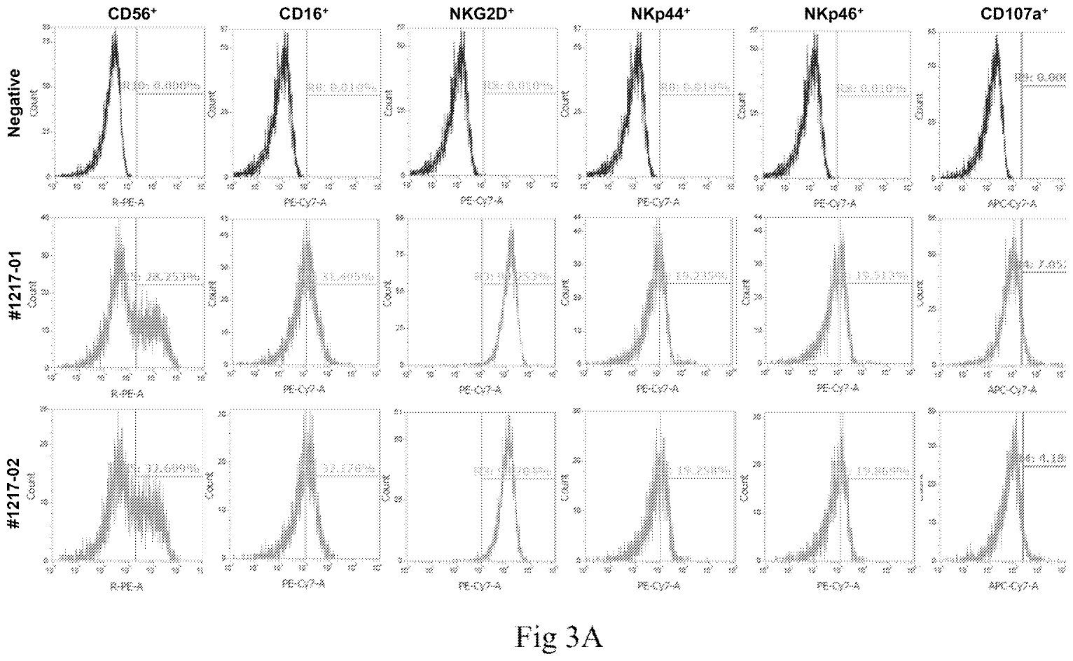

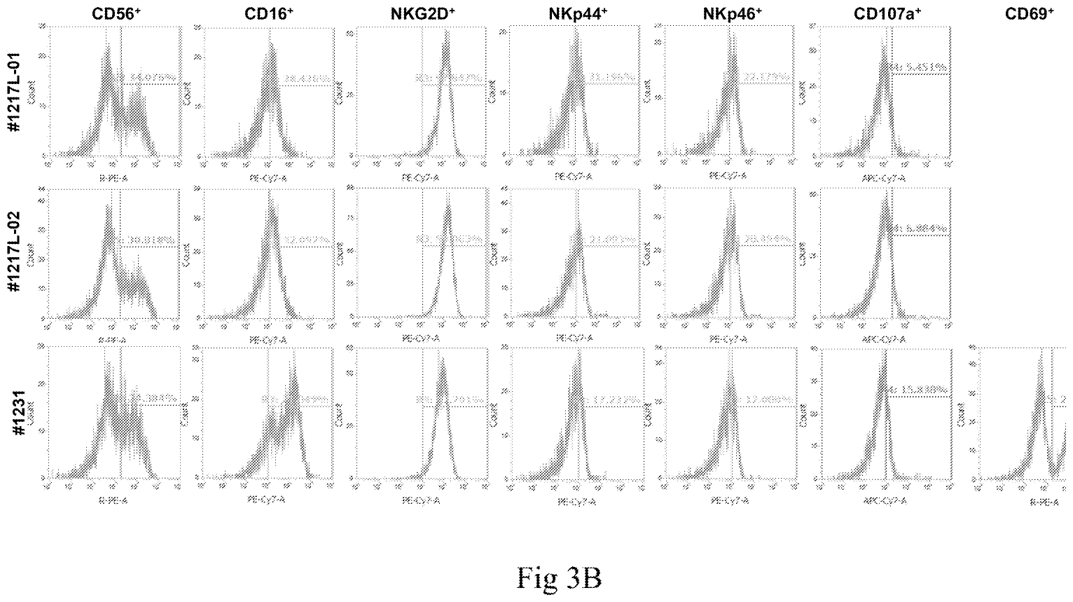

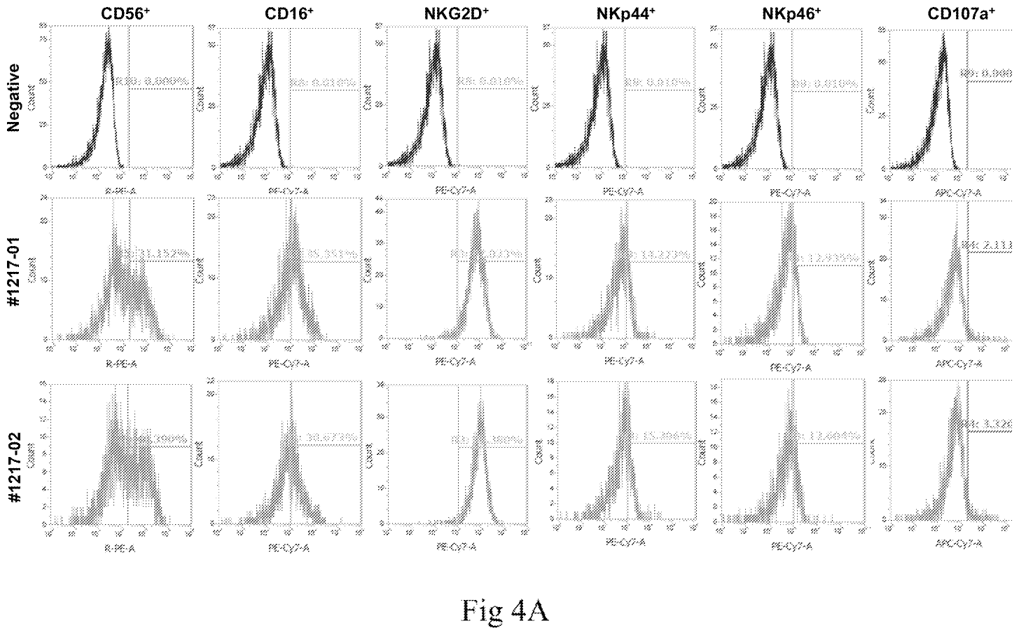

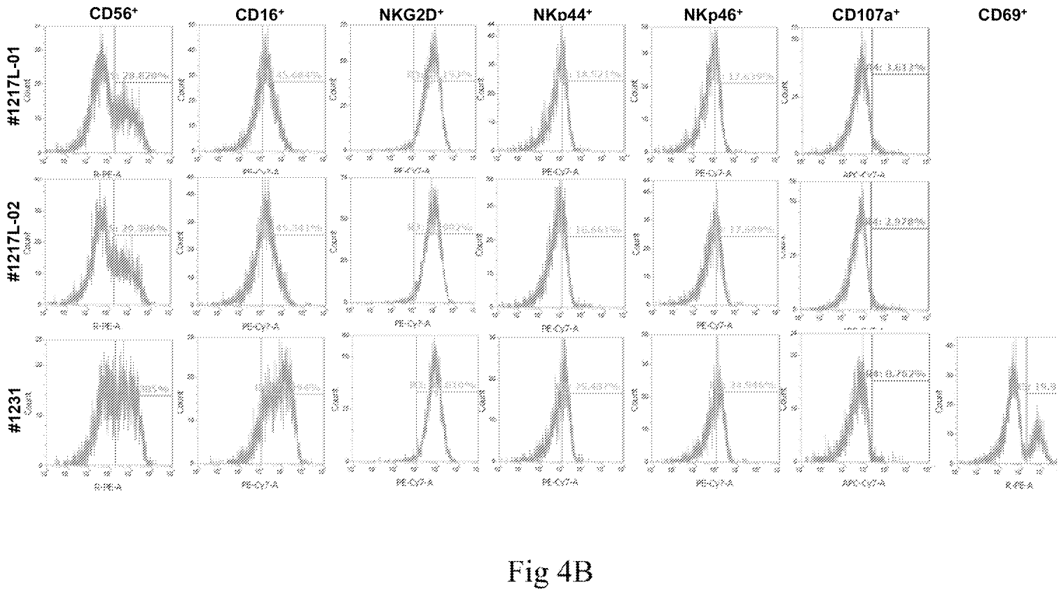

Please refer to FIG. 3A˜3B and FIG. 4A˜4B. FIG. 3A˜3B are histogram of gamma delta T cells collected on day 10. FIG. 4A˜4B are histogram of gamma delta T cells collected on day 14.

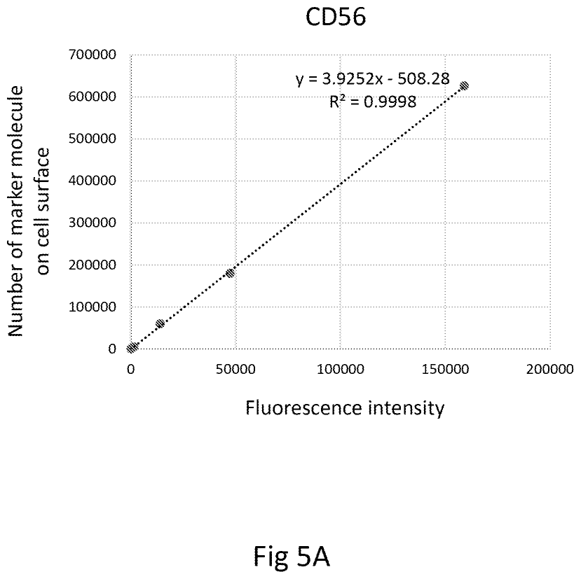

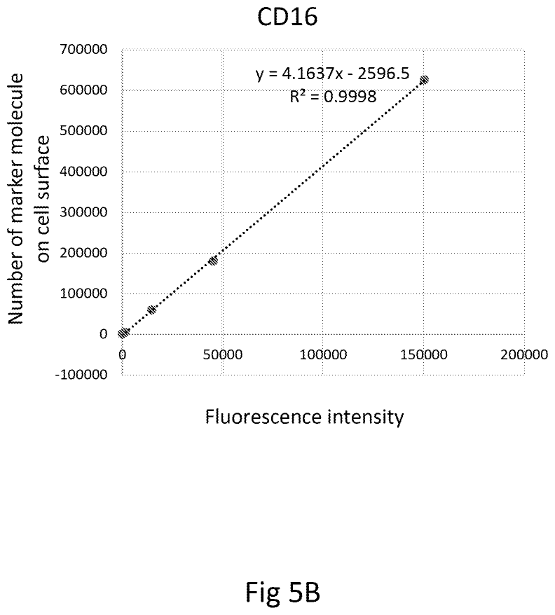

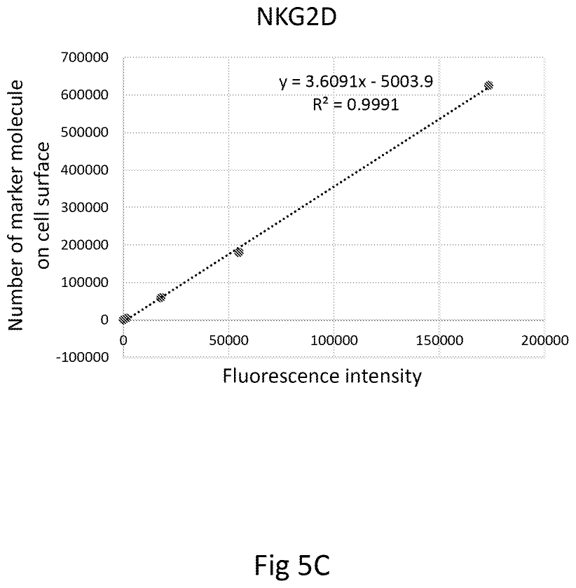

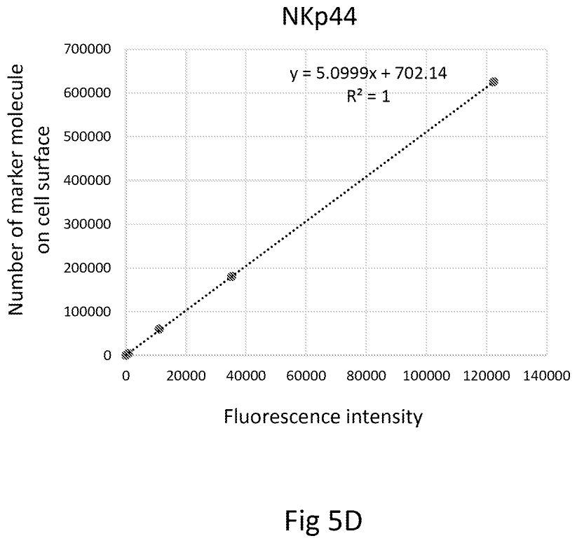

To convert mean fluorescence intensity of PE-conjugated mouse anti-human CD56 antibody (BioLegend, #304606), and PE-Cy7-conjugated mouse anti-human CD16 antibody (BioLegend, #331406), mouse anti-human NKG2D antibody (BioLegend, #320812), mouse anti-human NKp44 antibody (BioLegend, #325116), and mouse anti-human NKp46 antibody (BioLegend, #331916) in FIGS. 3A, 3B, 4A, and 4B into mean number of each receptor (CD56, CD16, NKG2D. NKp44 and NKp46) on cell surface, standard curves derived from Quantum™ Simply Cellular® kit (Bangs Laboratories, Inc, #815) were developed. There were 5 bottles of microspheres (4 populations “#1, #2, #3 and #4” coated with increasing amounts of anti-mouse IgG Fc antibody, 1 uncoated blank) in the Quantum™ Simply Cellular® kit. Ten microliter of anti-mouse IgG Fc antibody-bound microspheres, including #1, #2, #3 and #4, microsphere were individually incubated with 5 μg/mL of one of the corresponding antibodies (PE-conjugated mouse anti-human CD56, and PE-Cy7-conjugated mouse anti-human CD16, mouse anti-human NKG2D, mouse anti-human NKp44 or mouse anti-human NKp46 antibodies) in total 0.1 mL reaction volume at room temperature for 10 minutes. The blank microspheres were incubated without corresponding antibodies. Microspheres were washed with 0.5 mL of DPBS and the suspension was centrifuged at 400×g at room temperature for 5 minutes. The supernatant was removed and the suspended QSC microspheres were analyzed by flow cytometry. Acquired mean fluorescence intensity value of each microspheres was inserted into respective columns of manufacturer-provided calculation sheet (QuickCal V2.3) to generate the corresponding standard curve following manufacturer's instruction. After developing the standard curve (please refer to FIG. 5A˜5E), the mean fluorescence intensity value of the PE-conjugated mouse anti-human CD56 and PE-Cy7-conjugated mouse anti-human CD16, mouse anti-human NKG2D, mouse anti-human NKp44 or mouse anti-human NKp46 antibodies-stained γδT cells was next inserted to the QuickCal sheet to convert into corresponding numbers of each receptor on cell surface (please refer to Table 4).

From Table 4 to Table 6, five batches of γδT cells (i.e., #1217-01, #1217-02, #1217L-01, #1217L-02 and #1231) were put into NK-like marker profile analysis. In Table 4, the five batches of PBMC-derived γδT cells were expanded as described in the aforementioned expansion procedure. On Day 10, the percentage of γδT cells (CD3⁺ TCRVδ2⁺) in total T lymphocytes (CD3⁺) was within 50.28% and 89.50%. The percentage of γδT cells in total T lymphocytes on Day 14 resided within 32.84%˜91.00%. The percentage of T cells in total cells in both Days lies within 93.00%˜98.35%. With regards to cytotoxic function of natural killer cell (NK)-like γδT cells, pathogen recognition receptor CD56, antibody-dependent cellular cytotoxicity receptor CD16⁺, human natural cytotoxicity receptors NKG2D⁺, NKp44⁺ and NKp46⁺, degranulation marker CD107⁺ and early activation marker CD69⁺ expressions were analyzed. On Day 10, the percentage of CD56⁺, CD16⁺, NKG2D⁺, NKp14⁺ and NKp46⁺ population in the five batches of γδT cells ranged within 28.25%˜44.77%, 28.43%˜71.05%, 97.79%˜99.25%, 17.22%˜21.20% and 17.00%˜22.18%, respectively. The mean numbers of surface receptors on these batches of cells were 45297˜59450, 8486˜55095, 30722˜54176, 8790˜10943 and 7448˜9171, respectively. The percentage of CD107a⁺ population resided within 1.40%˜7.05%, whereas the percentage of CD69⁺ population in #1231 was 35.63%. On Day 14, the percentage of CD56⁺. CD16⁺, NKG2D⁺. NKp44⁺ and NKp46⁺ population in the five batches of γδT cells ranged within 28.82%˜56.49%, 30.67%˜72.99%, 96.19%˜97.38%, 14.22%˜25.49% and 12.60%˜24.95%, respectively. The mean number of surface receptors on these batches of cells were 32871˜55575, 11503˜54094, 28527˜36013, 9134˜10306 and 7108˜9392, respectively. The percentage of CD107a⁺ population resided within 0.76%˜3.61%, whereas the percentage of CD69⁺ population in #1231 was 24.82%.

In Table 5, #1217-01, #1217-02, #1217L-01, #1217L-02 and #1231 batches of PBMC-derived γδT cells exhibited no more than 5.21% and 7.23% of CD56⁺CD3⁻ (NK) populations in total T lymphocytes on Day 10 and Day 14, respectively. These five batches of cells present the percentage of CD3⁺Vαβ⁺ population in total T lymphocytes ranged from 4.18% to 40.07% and from 4.43%-54.84% on Day 10 and Day 14, respectively.

In γδT population subsets, there are naïve (CD45RA⁺CD27⁺), central memory (CD45RA⁻CD27⁺), effector memory (CD45RA⁻CD27⁻) and terminally differentiated (CD45RA⁺CD27⁻) populations. In Table 6, #1217-01, #1217-02, #1217L-01, #1217L-02 and #1231 batches of PBMC-derived γδT cells contained 8.72%˜36.91% of naïve, 7.45%˜23.87% of central memory, 10.93%˜38.64% of effector memory and 19.15%˜52.02% of terminally differentiated populations on Day 10. The percentage of naïve, central memory, effector memory and terminally differentiated populations of #1231 γδT cells on Day 14 was 40.76%, 17.81%, 9.79% and 31.63%, respectively.

Expression of NK cytotoxicity receptors (CD56, CD16, NKG2D. NKp44 and NKp46) and degranulation markers (CD107a) potentiates γδT cells with NK-like anti-tumor activity, whereas increase of effector memory and terminally differentiated populations helps γδT cells to localize at inflammatory microenvironment of tumor. CD69 expression represents activation of γδT cells.

It shows that the γδT cells in the present experiment express more cytotoxicity receptors (CD56, CD16. NKG2D. NKp44 and NKp46), more degranulation markers (CD107a), and more CD69.

TABLE 4
The biomarker of the cells obtained from Embodiment 1-2-1

| | % of T cells | % of CD3⁺Vδ2⁺ | % in total γδT cells/Mean no. of receptors on cells | | | | | | |
|---|---|---|---|---|---|---|---|---|---|
| | In total cells | In T lymphocytes | CD56⁺ | CD16⁺ | NKG2D⁺ | NKp44⁺ | NKp46⁺ | CD107a⁺ | CD69⁺ |
| Day 10 | | | | | | | | | |
| #1217-01 | 98.46 | 86.78 | 2 [symbol] .25/53925 | 31.50/9487 | 99.25/54176 | 19.24/10229 | 19.51/8135 | 7.05 | — |
| #1217-02 | [symbol] 4.75 | 55.67 | 32.70/45287 | 32.18/8486 | 9 [symbol] .80/43252 | 19.26/9843 | 19. [symbol] 7/8060 | 4.19 | — |
| #1217L-01 | 95.78 | 50.28 | 34.08/45 [symbol] 50 | 28.43/9287 | 97.85/45589 | 2 [symbol] .20/10943 | 22.18/9030 | 5.45 | — |
| #1217L-02 | 95.99 | 89.50 | 30.02/59450 | 32.10/9981 | 99.06/53781 | 2 [symbol] .09/10789 | 20.49/9171 | 6.88 | — |
| #1231 | [symbol] 3.95 | 7 [symbol] .19 | 44.77/56239 | 71.05/55095 | [symbol] 7.79/30722 | 17.22/8790 | 17.00/7448 | 1.40 | 35.63 |
| Day 14 | | | | | | | | | |
| #1217-01 | 96.58 | 42.64 | 31.15/32871 | 35.35/11503 | 97.02/28527 | 14.22/9288 | 12.94/7108 | 2.11 | — |
| #1217-02 | 96.24 | 32.84 | 40.39/43530 | 3 [symbol] .67/12639 | 97.38/34889 | 15.31/10033 | 12.60/8233 | 3.32 | — |
| #1217L-01 | 98.35 | 90.26 | 28.82/55575 | 45.68/13197 | 96.19/34739 | 18.52/9134 | 17.64/7967 | 3.61 | — |
| #1217L-02 | [symbol] 8.07 | [symbol] 1.00 | 29.40/51283 | 45.34/14430 | 96.90/36013 | 16.66/10080 | 17.70/822 [symbol] | 2.98 | — |
| #1231 | 93.00 | 73.98 | 56.49/39725 | 72.99/54094 | 97.01/31821 | 25.49/10306 | 24.95/9392 | 0.76 | 24.82 |

[symbol]

indicates data missing or illegible when filed

TABLE 5
The percentage of CD56⁺CD3⁻ and CD3⁺Vαβ⁺ populations in total
T lymphocytes obtained from Embodiment 1-2-1 (based on the number
of T lymphocytes as 100%)

| | % of CD56⁺CD3⁻ cells | % of CD3⁺Vαβ⁺ |
|---|---|---|
| | In total T lymphocytes | In total T lympphocytes |
| Day 10 | | |
| #1217-01 | 3.38 | 4.18 |
| #1217-02 | 2.16 | 34.99 |
| #1217L-01 | 2.47 | 40.07 |
| #1217L-02 | 3.59 | 4.93 |
| #1231 | 5.21 | 22.52 |
| Day 14 | | |
| #1217-01 | 2.05 | 36.21 |
| #1217-02 | 3.68 | 54.84 |
| #1217L-01 | 2.26 | 4.43 |
| #1217L-02 | 2.35 | 4.57 |
| #1231 | 7.23 | 18.69 |

TABLE 6
The percentage of different subpopulation in the CD3⁺yδ⁺T cells obtained from
Embodiment 1-2-1 (based on the number of CD3⁺yδ⁺T cells as 100%)

| | | Central | Effector | Terminally |
|---|---|---|---|---|
| | Naïve γδT | memory γδT | memory γδT | differentiated γδT |
| | (CD45RA⁺CD27⁺) | (CD45RA⁻CD27⁺) | (CD45RA⁻CD27⁻) | (CD45RA⁺CD27⁻) |
| Day 10 | | | | |
| #1217-01 | 8.72 | 7.45 | 31.67 | 62.02 |
| #1217-02 | 27.70 | 23.87 | 15.65 | 32.74 |
| #1217L-01 | 36.91 | 16.08 | 10.93 | 36.08 |
| #1217L-02 | 10.98 | 8.45 | 30.91 | 49.60 |
| #1231 | 25.15 | 16.98 | 38.64 | 19.15 |
| Day 14 | | | | |
| #1231 | 40.76 | 17.81 | 9.79 | 31.63 |

### Embodiment 1-2-3 Prepare Trastuzumab-Complexed Human Gamma Delta T Cells, Cetuximab-Complexed Human Gamma Delta T Cells, Rituximab-Complexed Human Gamma Delta T Cells, and Avelumab-Complexed Human Gamma Delta T Cells

The gamma delta T cell suspensions that was obtained by culturing for 14 days with the culture method disclosed in the Embodiment 1-2-1 (14-day gamma delta T cell suspension of the present invention, refer to as 14-day gamma delta T cell suspension) were used to prepare ingredient-complexed human gamma delta T cells. After binding ingredient such as Trastuzumab or cetuximab to cells in the 14-day gamma delta T cell suspension using a cell linker and an ingredient linker that are complementary, the trastuzumab-complexed human gamma delta T cell suspension, cetuximab-complexed human gamma delta T cells suspension, rituximab-complexed human gamma delta T cells suspension, or avelumab-complexed human gamma delta T cells suspension was obtained.

The procedure of binding ingredient (e.g., trastuzumab, cetuximab, rituximab, alemtuzumab, avelumab, daratumumab, elotuzumab, codrituzumab) to cells (e.g., all of the cells in the gamma delta T cell suspension in this experiment) was the same as that of Embodiment 1-1-4.

After the cell-ssDNA conjugate and the ingredient-ssDNA conjugate were mixed, the ingredient-complexed cells such as cells in the trastuzumab-complexed human gamma delta T cell suspension, cells in the cetuximab-complexed human gamma delta T cell suspension, cells in the rituximab-complexed human gamma delta T cells suspension, or cells in the avelumab-complexed human gamma delta T cells suspension were obtained.

### Embodiment 1-2-4 Cytotoxic Function of the Trastuzumab-Complexed Human Gamma Delta T Cells to Kill Trastuzumab-Responsive or Trastuzumab-Resistant Human Breast Cancer Cells

The experimental method of this embodiment is almost the same as that of Embodiment 1-1-5, except that the effector cell used in this Experiment were \textcircled{1} the cells in the 14-day gamma delta T cell suspension (Ctrl-gdT), or \textcircled{2} the cells in the trastuzumab-complexed human gamma delta T cell suspension (ACE-gdT-HER2).

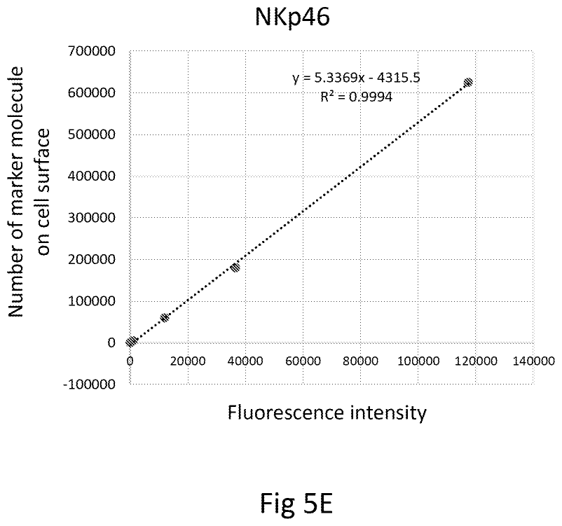

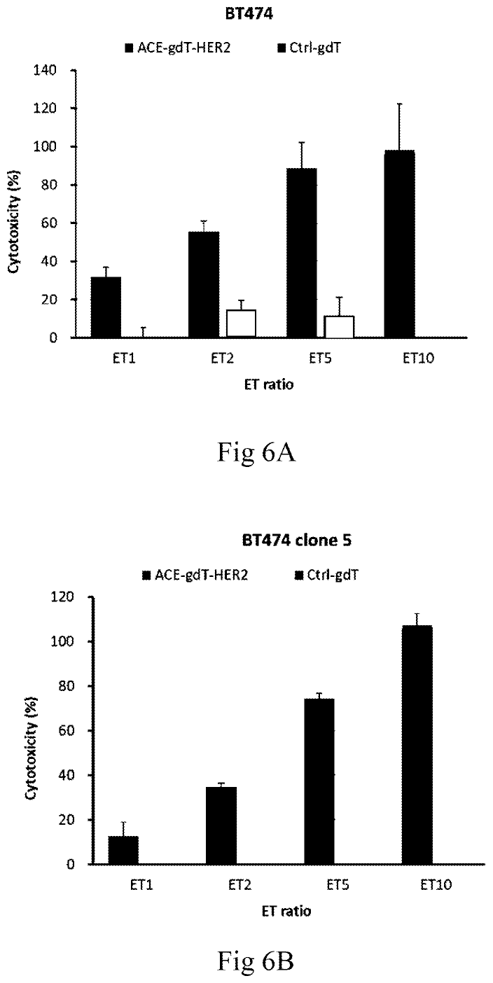

Please refer to FIGS. 6A and 6B. FIG. 6A is bar chart presenting the comparison of the cytotoxic function between the trastuzumab-complexed human gamma delta T cells and the non-complexed human gamma delta T cells to kill trastuzumab-responsive cancer cells (trastuzumab-responsive human breast cancer cell line BT474). FIG. 6B is a bar chart presenting the comparison of the cytotoxic function between the trastuzumab-complexed human gamma delta T cells and the non-complexed human gamma delta T cells to kill trastuzumab-resistant cancer cells (trastuzumab-resistant human breast cancer cell line BT474 Clone 5). As shown in FIGS. 6A and 6B, the ability of human gamma delta T cells (Ctrl-gdT) to kill trastuzumab-responsive human breast cancer cell line BT474 and trastuzumab-resistant human breast cancer cell line BT474 Clone 5 were similar. Unexpectedly, the killing capacity of trastuzumab-complexed human gamma delta T cells (ACE-gdT-HER2, which is human gamma delta T cell complexed with trastuzumab) to trastuzumab-responsive human breast cancer cell line BT474 and trastuzumab-resistant human breast cancer cell line BT474 Clone 5 were also similar.

According to the experimental results of FIG. 6A and FIG. 6B as well as FIG. 1A, FIG. 1B, and FIG. 2, inventors of the present invention expect that:

Trastuzumab can only kill a small amount of trastuzumab-resistant cancer cells, and the killing effect of human gamma delta T cells co-cultured with trastuzumab (Ctrl-gdT+trastuzumab) is not significantly higher than the effect of human gamma delta T cells (Ctrl-gdT). Unexpectedly, the killing effect of human gamma delta T cells complexed with trastuzumab (ACE-gdT-HER2, which is human gamma delta T cell complexed with trastuzumab) is significantly higher than the killing effect of human gamma delta T cells (Ctrl-gdT). This is an unexpected result and indicates that trastuzumab and human gamma delta T cells (Ctrl-gdT) in the trastuzumab-complexed human gamma delta T cells manifest synergy in killing trastuzumab-resistant cancer cells.

Therefore, ingredient-complexed cytotoxic cells such as trastuzumab-complexed human gamma delta T cells (ACE-gdT-HER2) obtained by complexing trastuzumab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as trastuzumab-complexed human gamma delta T cells are able to solve the problem that no drug can cure abnormal cells that are resistant, refractory, insensitive, non-responsive, or inadequately responsive to current FDA-approved drugs (such as FDA-approved trastuzumab), as well as improving the treatment effectiveness of an ingredient in a subject who is resistant, refractory, insensitive, non-responsive, or inadequately responsive to the ingredient.

### Embodiment 1-2-5 Cytotoxic Function of the Cetuximab-Complexed Human Gamma Delta T Cells to Kill Cetuximab-Responsive or Cetuximab-Resistant Human Cancer Cells

The experimental method of this embodiment is almost the same as that of Experiment 1-1-6, except that the effector cell used in this Experiment were \textcircled{1} the cells in the 16-day gamma delta T cell suspension (Ctrl-gdT), or \textcircled{2} the cells in the cetuximab-complexed human gamma delta T cell suspension (ACE2016).

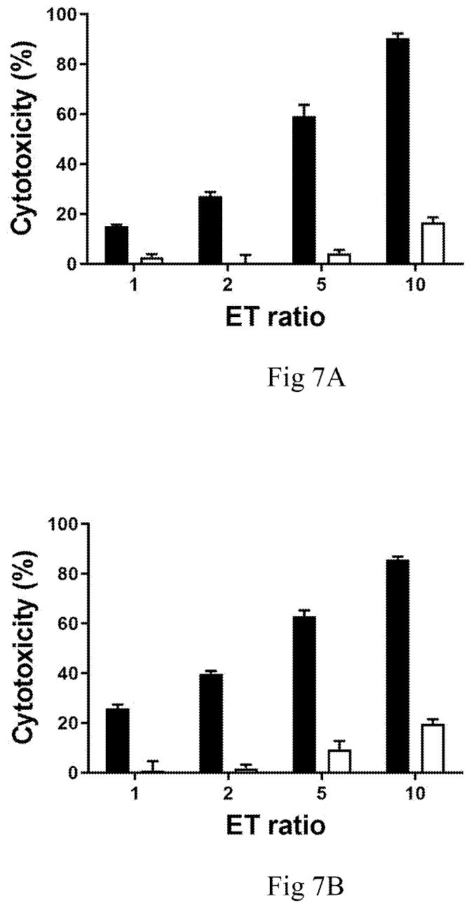

Please refer to FIGS. 7A and 7B. FIG. 7A is bar chart presenting the comparison of the cytotoxic function between the cetuximab-complexed human gamma delta T cells and the non-complexed human gamma delta T cells to kill cetuximab-responsive cancer cells (cetuximab-responsive human adenocarcinoma cell line HCC827). FIG. 7B is a bar chart presenting the comparison of the cytotoxic function between the cetuximab-complexed human gamma delta T cells and the non-complexed human gamma delta T cells to kill cetuximab-resistant cancer cells (cetuximab-resistant human colorectal adenocarcinoma cell line HT-29). As shown in FIGS. 7A and 7B, the ability of human gamma delta T cells (Ctrl-gdT) to kill cetuximab-responsive human adenocarcinoma cell line HCC827 and cetuximab-resistant human colorectal adenocarcinoma cell line HT-29 were similar. Unexpectedly, the killing capacity of cetuximab-complexed human gamma delta T cells (ACE2016, which is human gamma delta T cell complexed with cetuximab) to cetuximab-responsive human adenocarcinoma cell line HCC827 and cetuximab-resistant human colorectal adenocarcinoma cell line HT-29 were also similar.

According to the experimental results of FIG. 7A and FIG. 7B as well as FIG. 1A. FIG. 1B, and FIG. 2, inventors of the present invention expect that:

cetuximab can only kill a small amount of cetuximab-resistant cancer cells, and the killing effect of human gamma delta T cells co-cultured with cetuximab (Ctrl-gdT+cetuximab) is not significantly higher than the effect of human gamma delta T cells (Ctrl-gdT). Unexpectedly, the killing effect of human gamma delta T cells complexed with cetuximab (ACE2016, which is human gamma delta T cell complexed with cetuximab) is significantly higher than the killing effect of human gamma delta T cells (Ctrl-gdT). This is an unexpected result and indicates that cetuximab and human gamma delta T cells (Ctrl-gdT) in the cetuximab-complexed human gamma delta T cells manifests synergy in killing cetuximab-resistant cancer cells.

Therefore, ingredient-complexed cytotoxic cells such as cetuximab-complexed human gamma delta T cells obtained by complexing cetuximab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as cetuximab-complexed human gamma delta T cells is able to solve the problem that no drug can cure abnormal cells that are resistant, refractory, insensitive, non-responsive, or inadequately responsive to current FDA-approved drugs (such as FDA-approved cetuximab), as well as improving the treatment effectiveness of an ingredient in a subject who is resistant, refractory, insensitive, non-responsive, or inadequately responsive to the ingredient.

### Embodiment 1-2-6 Cytotoxic Function of the Cetuximab-Complexed Human Gamma Delta T Cells to Kill Cetuximab-Responsive or Cetuximab-Resistant Human Tongue Cancer Cells

The experimental method of this embodiment is almost the same as that of Embodiment 1-1-7, except that the effector cell used in this Experiment were \textcircled{1} the cells in the 16-day gamma delta T cell suspension, or \textcircled{2} the cells in the cetuximab-complexed human gamma delta T cell suspension. Inventors of the present invention expect that:
(1) The ability of human gamma delta T cells (Ctrl-gdT) to kill cetuximab-responsive human tongue squamous carcinoma cell line HSC-4 and cetuximab-resistant human tongue squamous carcinoma cell line SAS were similar. Unexpectedly, the killing capacity of cetuximab-complexed human gamma delta T cells to cetuximab-responsive human tongue squamous carcinoma cell line HSC-4 and cetuximab-resistant human tongue squamous carcinoma cell line SAS were also similar;
(2) cetuximab can only kill a small amount of cetuximab-resistant cancer cells, and the killing effect of human gamma delta T cells co-cultured with cetuximab (Ctrl-gdT+cetuximab) is not significantly higher than the effect of human gamma delta T cells (Ctrl-gdT). Unexpectedly, the killing effect of human gamma delta T cells complexed with cetuximab is significantly higher than the killing effect of human gamma delta T cells (Ctrl-gdT). This is an unexpected result and indicates that cetuximab and human gamma delta T cell (Ctrl-gdT) in the cetuximab-complexed human gamma delta T cells manifests synergy in killing cetuximab-resistant cancer cells.

Therefore, ingredient-complexed cytotoxic cells such as cetuximab-complexed human gamma delta T cells obtained by complexing cetuximab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as cetuximab-complexed human gamma delta T cells is able to solve the problem that no drug can cure abnormal cells that are resistant, refractory, insensitive, non-responsive, or inadequately responsive to current FDA-approved drugs (such as FDA-approved cetuximab), as well as improving the treatment effectiveness of an ingredient in a subject who is resistant, refractory, insensitive, non-responsive, or inadequately responsive to the ingredient.

### Embodiment 1-2-7 Cytotoxic Function of the Rituximab-Complexed Human Gamma Delta T Cells to Kill Rituximab-Responsive or Rituximab-Resistant Human Lymphoma Cells

CellTiter-Glo® Luminescent Cell Viability Assay (Promega, USA) was used in this embodiment to detect the cytotoxic ability of the cultured effector cells toward target cells. First, wells in a CELLSTAR® 96 well plates (Cat. Number 655083, purchased from Greiner) were divided into:
(1) target control well.
(2) rituximab basal well.
(3) rituximab and target experimental well.
(4) Ctrl-gdT basal well.
(5) Ctrl-gdT and target experimental well.
(6) Ctrl-gdT and rituximab basal well.
(7) Ctrl-gdT and rituximab and target cell experimental well.
(8) ACE-gdT basal well, and
(9) ACE-gdT and target cell experimental well.

The effector cell used in this Experiment were \textcircled{1} the cells in the 16-day gamma delta T cell suspension, or \textcircled{2} the cells in the rituximab-complexed human gamma delta T cell suspension; wherein the rituximab is an antibody against CD20 protein with product name as Mabthera (purchased from Creative BioLabs. Roche. Amgen, Phizer).

The target cells used in this Experiment were \textcircled{1} suspended human Burkitts lymphoma cell line Raji (CCL-86, purchased from ATCC) that is not resistant to rituximab, or \textcircled{2} rituximab-resistant human lymphoma cell line Raji-2R80 (inventors of the present invention established this rituximab-resistant clones Raji-2R80 from Raji; the method of developing the rituximab-resistant clones are known, or will be apparent, to those skilled in the art, such as develop according to reference (1) Czuczman et al. Clin. Cancer Res. (2008): 14:1561.-1570 (10.1158/1078-0432.CCR-07-1254); or (2) Jazirehi et al. Cancer Res. (2007); 67:1270-1281 (10.1158/0008-5472.CAN-06-2184).

Target cells (Raji or Raji-2R80 cells) were seeded in:
the target control wells.
the rituximab and target experimental wells.
the Ctrl-gdT and target experimental wells.
the Ctrl-gdT and rituximab and target cell experimental wells, and
the ACE-gdT and target cell experimental wells.
so that each well-contained 5000 target cells.

A sample of the rituximab-complexed human gamma delta T cell suspension (ACE-gdT-CD20) was added to:
the ACE-gdT basal wells, and
the ACE-gdT and target cell experimental wells.
so that the ratio of the number of effector cell to the number of target cell (Raji or Raji-2R80 cells) was 2, 5, or 10 respectively.

A sample of the 16-day gamma delta T cell suspension (Ctrl-gdT) was added to:
the Ctrl-gdT basal wells.
the Ctrl-gdT and target experimental wells.
the Ctrl-gdT and rituximab basal wells, and
the Ctrl-gdT and rituximab and target cell experimental wells.
so that the ratio of the number of effector cell to the number of target cell (Raji or Raji-2R80 cells) was 2, 5, or 10 respectively.

0.8, 2, or 4 ng of rituximab was added to the “Ctrl-gdT and rituximab basal wells”, and the “Ctrl-gdT and rituximab and target cell experimental wells” respectively. Therefore, the amount of rituximab in the “Ctrl-gdT and rituximab basal wells” was respectively same as the total amount of the rituximab linked to the cells in the “ACE-gdT basal wells” in which the ratio of the number of effector cell to the number of target cell was 2, 5, or 10; and the amount of rituximab in the “Ctrl-gdT and rituximab and target cell experimental wells” was respectively same as the total amount of the rituximab linked to the cells in the “ACE-gdT and target cell experimental wells” in which the ratio of the number of effector cell to the number of target cell was 2, 5, or 10.

The CELLSTAR® 96 well plates were incubated at 37° C. in 5% CO₂ for 4 hours. After 4 hours of incubation, the culture were mixed with 50 μL of CellTiter-Glo® substrate (provided in the CellTiter-Glo® luminescent cell viability assay kit. Promega. Cat. G7570) and incubated at room temperature without light for 12 minutes. The luminescence of each well was measured and recorded by luminescence plate reader (Synergy H1. BioTek Instruments, USA).

Wherein, the greater the number of live cells remained in the well, the higher the luminescence detected by the Synergy H1 system. Therefore, the luminescence can be used to convert the percentage of target cells that are lysed in the experimental well. The formula used to convert the luminescence to the percentage of target cells that are lysed in the experimental wells are as follows:

Percentage of lysed target cell in “rituximab and target experimental well” (%)=1−[(luminescence of rituximab and target experimental well−luminescence of rituximab basal well)÷(luminescence of target control well)]×100%

Percentage of lysed target cell in “Ctrl-gdT and target experimental well” (%)=1−[(luminescence of Ctrl-gdT and target experimental well−luminescence of Ctrl-gdT basal well)÷(luminescence of target control well)]×100%

Percentage of lysed target cell in “Ctrl-gdT and rituximab and target cell experimental well” (%)=1−[(luminescence of Ctrl-gdT and rituximab and target cell experimental well−luminescence of Ctrl-gdT and rituximab basal well)÷(luminescence of target control well)]×100%

Percentage of lysed target cell in “ACE-gdT and target cell experimental well” (%)=1−[(luminescence of ACE-gdT and target cell experimental well−luminescence of ACE-gdT basal well)=(luminescence of target control well)]×100%

Inventors of the present invention expect that:
(1) The ability of human gamma delta T cells (Ctrl-gdT) to kill rituximab-responsive Raji cells and rituximab-resistant Raji-2R80 cells were similar. Unexpectedly, the killing capacity of rituximab-complexed human gamma delta T cells to rituximab-responsive Raji cells and rituximab-resistant Raji-2R80 cells were also similar;
(2) rituximab can only kill a small amount of rituximab-resistant cancer cells, and the killing effect of human gamma delta T cells co-cultured with rituximab (Ctrl-gdT+rituximab) is not significantly higher than the effect of human gamma delta T cells (Ctrl-gdT). Unexpectedly, the killing effect of human gamma delta T cells complexed with rituximab is significantly higher than the killing effect of human gamma delta T cells (Ctrl-gdT). This is an unexpected result and indicates that rituximab and human gamma delta T cell (Ctrl-gdT) in the rituximab-complexed human gamma delta T cells manifests synergy in killing rituximab-resistant cancer cells.

Therefore, ingredient-complexed cytotoxic cells such as rituximab-complexed human gamma delta T cells obtained by complexing rituximab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as rituximab-complexed human gamma delta T cells is able to solve the problem that no drug can cure abnormal cells that are resistant, refractory, insensitive, non-responsive, or inadequately responsive to current FDA-approved drugs (such as FDA-approved rituximab), as well as improving the treatment effectiveness of an ingredient in a subject who is resistant, refractory, insensitive, non-responsive, or inadequately responsive to the ingredient.

### Embodiment 1-2-8 Cytotoxic Function of the Rituximab-Complexed Human Gamma Delta T Cells to Kill Normal or Rituximab-Resistant Human Lymphoma Cells

The experimental method of this embodiment is almost the same as that of Embodiment 1-2-7, except that: the target cells used in this Experiment were \textcircled{1} human lymphoma cell line Daudi (CCL-213, purchased from ATCC) that is not resistant to rituximab, or \textcircled{2} rituximab-resistant human lymphoma cell line Daudi-RR (inventors of the present invention established this rituximab-resistant clones Daudi-RR from Daudi; the method of developing the rituximab-resistant clones are known, or will be apparent, to those skilled in the art, such as develop according to reference (1) Czuczman et al. Clin. Cancer Res. (2008): 14:1561-1570 (10.1158/1078-0432.CCR-07-1254); or (2) Jazirehi et al. Cancer Res. (2007); 67:1270-1281 (10.1158/0008-5472.CAN-06-2184).

Inventors of the present invention expect that:
(1) The ability of human gamma delta T cells (Ctrl-gdT) to kill rituximab-responsive Daudi cells and rituximab-resistant Daudi-RR cells were similar. Unexpectedly, the killing capacity of rituximab-complexed human gamma delta T cells to rituximab-responsive Daudi cells and rituximab-resistant Daudi-RR cells were also similar;
(2) rituximab can only kill a small amount of rituximab-resistant cancer cells, and the killing effect of human gamma delta T cells co-cultured with rituximab (Ctrl-gdT+rituximab) is not significantly higher than the effect of human gamma delta T cells (Ctrl-gdT). Unexpectedly, the killing effect of human gamma delta T cells complexed with rituximab is significantly higher than the killing effect of human gamma delta T cells (Ctrl-gdT). This is an unexpected result and indicates that rituximab and human gamma delta T cell (Ctrl-gdT) in the rituximab-complexed human gamma delta T cells manifests synergy in killing rituximab-resistant cancer cells.

Therefore, ingredient-complexed cytotoxic cells such as rituximab-complexed human gamma delta T cells obtained by complexing rituximab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as rituximab-complexed human gamma delta T cells is able to solve the problem that no drug can cure abnormal cells that are resistant, refractory, insensitive, non-responsive, or inadequately responsive to current FDA-approved drugs (such as FDA-approved rituximab), as well as improving the treatment effectiveness of an ingredient in a subject who is resistant, refractory, insensitive, non-responsive, or inadequately responsive to the ingredient.

### Embodiment 1-2-9 Cytotoxic Function of the Avelumab-Complexed Human Gamma Delta T Cells to Kill Avelumab-Responsive or Avelumab-Resistant Human Breast Cancer Cells

The experimental method of this embodiment is almost the same as that of Embodiment 1-2-7, except that:
(1) the effector cell used in this Experiment were \textcircled{1} the cells in the 16-day gamma delta T cell suspension, or \textcircled{2} the cells in the avelumab-complexed human gamma delta T cell suspension, wherein the avelumab is an antibody against PD-L1 protein with product name as Bavencio R (purchased from Merck KGaA. Pfizer); and
(2) the target cells used in this Experiment were \textcircled{1} human breast cancer cell line MDA-MB-231 (HTB-26, purchased from ATCC) that is not resistant to avelumab, or \textcircled{2} avelumab-resistant human breast cancer cell line MDA-MB-468 (HTB-132, purchased from ATCC).

Inventors of the present invention expect that:
(1) The ability of human gamma delta T cells (Ctrl-gdT) to kill avelumab-responsive human breast cancer cell line MDA-MB-231 and avelumab-resistant human breast cancer cell line MDA-MB-468 were similar. Unexpectedly, the killing capacity of avelumab-complexed human gamma delta T cells to avelumab-responsive human breast cancer cell line MDA-MB-231 and avelumab-resistant human breast cancer cell line MDA-MB-468 were also similar;
(2) avelumab can only kill a small amount of avelumab-resistant cancer cells, and the killing effect of human gamma delta T cells co-cultured with avelumab (Ctrl-gdT+avelumab) is not significantly higher than the effect of human gamma delta T cells (Ctrl-gdT). Unexpectedly, the killing effect of human gamma delta T cells complexed with avelumab is significantly higher than the killing effect of human gamma delta T cells (Ctrl-gdT). This is an unexpected result and indicates that avelumab and human gamma delta T cell (Ctrl-gdT) in the avelumab-complexed human gamma delta T cells manifests synergy in killing avelumab-resistant cancer cells.

Therefore, ingredient-complexed cytotoxic cells such as avelumab-complexed human gamma delta T cells obtained by complexing avelumab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as avelumab-complexed human gamma delta T cells is able to solve the problem that no drug can cure abnormal cells that are resistant, refractory, insensitive, non-responsive, or inadequately responsive to current FDA-approved drugs (such as FDA-approved avelumab), as well as improving the treatment effectiveness of an ingredient in a subject who is resistant, refractory, insensitive, non-responsive, or inadequately responsive to the ingredient.

### Embodiment 1-2-10 Cytotoxic Function of the Avelumab-Complexed Human Gamma Delta T Cells to Kill Normal or Avelumab-Resistant Human Lung Cancer Cells

The experimental method of this embodiment is almost the same as that of Embodiment 1-2-9, except that: the target cells used in this Experiment were \textcircled{1} human lung cancer cell line H1650 (CRL-5883, purchased from ATCC) that is not resistant to avelumab, or \textcircled{2} avelumab-resistant human lung cancer cell line H2087 (CRL-5922, purchased from ATCC).

Inventors of the present invention expect that:
(1) The ability of human gamma delta T cells (Ctrl-gdT) to kill avelumab-responsive human lung cancer cell line H1650 and avelumab-resistant human lung cancer cell line H2087 were similar. Unexpectedly, the killing capacity of avelumab-complexed human gamma delta T cells to normal human lung cancer cell line H1650 and avelumab-resistant human lung cancer cell line H2087 were also similar;
(2) avelumab can only kill a small amount of avelumab-resistant cancer cells, and the killing effect of human gamma delta T cells co-cultured with avelumab (Ctrl-gdT+avelumab) is not significantly higher than the effect of human gamma delta T cells (Ctrl-gdT). Unexpectedly, the killing effect of human gamma delta T cells complexed with avelumab is significantly higher than the killing effect of human gamma delta T cells (Ctrl-gdT). This is an unexpected result and indicates that avelumab and human gamma delta T cell (Ctrl-gdT) in the avelumab-complexed human gamma delta T cells manifests synergy in killing avelumab-resistant cancer cells.

Therefore, ingredient-complexed cytotoxic cells such as avelumab-complexed human gamma delta T cells obtained by complexing avelumab and human gamma delta T cells (Ctrl-gdT) can be used for treating abnormal cells that are resistant, refractory, insensitive, non-responsive, or inadequately responsive to the ingredient, so that ingredient-complexed cytotoxic cells such as avelumab-complexed human gamma delta T cells is able to solve the problem that no drug can cure abnormal cells that are resistant, refractory, insensitive, non-responsive, or inadequately responsive to current FDA-approved drugs (such as FDA-approved avelumab), as well as improving the treatment effectiveness of an ingredient in a subject who is resistant, refractory, insensitive, non-responsive, or inadequately responsive to the ingredient.

### Embodiment 2: Activation Marker and Cytotoxic Molecule Expression of the Ingredient-Complexed Cytotoxic Cells after Co-Culture with Ingredient-Responsive or Ingredient-Resistant Target Cells

CD107a is an activation marker, and IFN gamma, TNF alpha, and granzyme B are cytotoxic molecules. Therefore, the expression of CD107a, IFN gamma, TNF alpha, and granzyme B in cytotoxic cells are used to indicate the activation state of the cytotoxic cells.

### Embodiment 2-1: Activation Marker and Cytotoxic Molecule Expression of the Ingredient-Complexed Human CD16⁺ Natural Killer Cells after Co-Culture with Ingredient-Responsive or Ingredient-Resistant Target Cells

### Embodiment 2-1-1 Activation Marker and Cytotoxic Molecule Expression of the Trastuzumab-Complexed Human CD16⁺ Natural Killer Cells after Co-Culture with Trastuzumab-Responsive or Trastuzumab-Resistant Human Breast Cancer Cells

This embodiment comprised a 96-well cell culture plate to carry out cytotoxicity test, and the wells in 96-well cell culture plate were divided into ACE1702 ET2 experimental wells, ACE1702 ET5 experimental wells. ACE1702 ET10 experimental wells. Ctrl-oNK ET2 experimental wells. Ctrl-oNK ET5 experimental wells. Ctrl-oNK ET10 experimental wells. Ctrl-oNK and Trastuzumab ET2 experimental wells, Ctrl-oNK and Trastuzumab ET5 experimental wells. Ctrl-oNK and Trastuzumab ET10 experimental wells, target control wells and medium background control wells.

The effector cells used in this embodiment were the cells in the 32-day cultured oNK cell suspension or the cells in the Trastuzumab-complexed human CD16⁺ natural killer cell suspension, and the target cells were sensitive BT-474 (HTB-20, purchased from ATCC) or resistant BT-474 clone 5 cell lines (CRL-3247, purchased from ATCC), which are adherent human breast cancer cell lines.

BT-474 or BT-474 clone 5 target cells were seeded in the ACE1702 ET2 experimental wells, ACE1702 ET5 experimental wells, ACE1702 ET10 experimental wells. Ctrl-oNK ET2 experimental wells, Ctrl-oNK ET5 experimental wells. Ctrl-oNK ET10 experimental wells. Ctrl-oNK and Trastuzumab ET2 experimental wells. Ctrl-oNK and Trastuzumab ET5 experimental wells. Ctrl-oNK and Trastuzumab ET10 experimental wells, and target control wells: hence, each well-contained 10000 target cells, and was allowed to sit 30 minutes, then the cell culture plate was incubated under the condition of 37° C. and 5% carbon dioxide for two hours.

A sample of the 16-day cultured oNK cell suspension (Ctrl-oNK) or the Trastuzumab-complexed human CD16⁺ natural killer cell suspension (ACE1702) was added to the ACE1702 ET2 experimental wells. ACE1702 ET5 experimental wells, ACE1702 ET10 experimental wells, Ctrl-oNK ET2 experimental wells. Ctrl-oNK ET5 experimental wells. Ctrl-oNK ET10 experimental wells, Ctrl-oNK and Trastuzumab ET2 experimental wells, Ctrl-oNK and Trastuzumab ET5 experimental wells. Ctrl-oNK and Trastuzumab ET10 experimental wells, and the ratio of the number of effector cells to the number of target cells (target cells) was 2, 5 and 10. The cell culture plate was placed in the incubator under the condition of 37° C. and 5% carbon dioxide for 5 hours.

The 96-well cell culture plate was centrifuged at 400×g for 5 minutes. The supernatants were removed and the cell pellets were washed with 0.2 mL of DPBS. Washed cell pellets were then stained with 100 uL of DPBS containing FITC-anti-human TNF alpha antibody (BioLegend, Cat.502906), anti-PE-anti-human CD56 antibody (BioLegend. Cat.), PE/Cy7-anti-human IFN gamma antibody (BioLegend, Cat.502528), Alexa Fluor 647-anti-human granzyme B antibody (BioLegend, Cat.), and APC-Cy7-anti-human CD107a antibody (BioLegend, Cat.328630) at 1:50 dilution for 10 minutes. The stained cells were centrifuged and washed with 0.2 mL of DPBS. The washed cells were resuspended with 0.5 mL of DPBS, and the CD56-positive gated populations were further analyzed for the percentage of TNF-α⁺, IFN-γ⁺, granzyme B⁺ and CD107a⁺. The mean fluorescence intensity of CD56-positive gated populations was also analyzed.

inventors of the present invention expect that:
(1) When co-cultured with BT-474 cancer cells or BT-474 clone 5 cancer cells, human CD16⁺ natural killer cells (Ctrl-oNK) express only a small amount of TNF-α, IFN-γ, granzyme B and CD107a.
(2) When co-cultured with BT-474 cancer cells with addition of Trastuzumab, human CD16⁺ natural killer cells (Ctrl-oNK) would express more TNF alpha, IFN gamma, granzyme B and CD107a. However, when co-cultured with BT-474 clone 5 cancer cells with addition of Trastuzumab, human CD16⁺ natural killer cells (Ctrl-oNK) would not express as many TNF alpha, IFN gamma, granzyme B and CD107a as when co-cultured with BT-474 cancer cells with addition of Trastuzumab. This result indicates that in the case of BT-474 cells, addition of Trastuzumab is able to highly activate human CD16⁺ natural killer cells (Ctrl-oNK) to destroy BT-474 tumor cells through mechanisms such as antibody-dependent cell cytotoxicity (ADCC); however, in the case of BT-474 clone 5 cells, addition of Trastuzumab failed to highly activate human CD16⁺ natural killer cells (Ctrl-oNK) to destroy Trastuzumab-resistant BT-474 clone 5 tumor cells through mechanisms such as antibody-dependent cell cytotoxicity (ADCC).
(3) Unexpectedly, whether it was co-cultured with BT-474 or BT-474 clone 5 cancer cells. Trastuzumab-complexed human CD16⁺ natural killer cells (ACE1702) express large amounts of TNF alpha. IFN gamma, granzyme B and CD107a, and the expression levels of various molecules such as TNF alpha. IFN gamma, granzyme B and CD107a are similar in both groups. A significant enhancement of cytotoxicity of ACE1702 against both BT-474 and BT-474 clone 5 compared to activation of human CD16⁺ natural killer cells (Ctrl-oNK) through equivalent amount of Trastuzumab was observed. These results indicate that as compare with addition of Trastuzumab (failed to highly activate human CD16⁺ natural killer cells to destroy Trastuzumab-resistant BT-474 clone 5 tumor cells), complexed Trastuzumab is able to highly activate Trastuzumab-complexed human CD16⁺ natural killer cells (ACE1702) to destroy both of BT-474 tumor cells and Trastuzumab-resistant BT-474 clone 5 tumor cells through mechanisms such as antibody-dependent cell cytotoxicity (ADCC).

### Embodiment 2-1-2 Activation Marker and Cytotoxic Molecule Expression of the Cetuximab-Complexed Human CD16⁺ Natural Killer Cells after Co-Culture with Cetuximab-Responsive or Cetuximab-Resistant Human Colon Cancer Cells

The experimental method of this embodiment is almost the same as that of Embodiment 2-1-1, except that:
(1) the effector cell used in this Experiment were \textcircled{1} the cells in the 45-day cultured oNK cell suspension, or \textcircled{2} the cells in the cetuximab-complexed human CD16⁺ natural killer cell suspension; and
(2) the target cells used in this Experiment were \textcircled{1} human colorectal adenocarcinoma cell line HCC827-luc (JCRB1516, purchased from JCRB) that is not resistant to cetuximab, or \textcircled{2} cetuximab-resistant human colorectal adenocarcinoma cell line HT-29 (HTB-38, purchased from ATCC).

inventors of the present invention expect that:
(1) Whether co-cultured with NCI-H508 cancer cells or HT-29 cancer cells, human CD16⁺ natural killer cells (Ctrl-oNK) express only a small amount of TNF alpha, IFN gamma, granzyme B and CD107a.
(2) When co-cultured with HCC827-luc cancer cells with addition of cetuximab, human CD16⁺ natural killer cells (Ctrl-oNK) would express more TNF alpha, IFN gamma, granzyme B and CD107a. However, when co-cultured with HT-29 cancer cells with addition of cetuximab, human CD16⁺ natural killer cells (Ctrl-oNK) would not express as many TNF alpha, IFN gamma, granzyme B and CD107a as when co-cultured with HCC827-luc cancer cells with addition of cetuximab. This result indicates that in the case of HCC827-luc cells, addition of cetuximab is able to highly activate human CD16⁺ natural killer cells (Ctrl-oNK) to destroy HCC827-luc tumor cells through mechanisms such as antibody-dependent cell cytotoxicity (ADCC); however, in the case of HT-29 cells, addition of cetuximab failed to highly activate human CD16⁺ natural killer cells (Ctrl-oNK) to destroy cetuximab-resistant HT-29 tumor cells through mechanisms such as antibody-dependent cell cytotoxicity (ADCC).
(3) Unexpectedly, whether it was co-cultured with HCC827-luc or HT-29 cancer cells, cetuximab-complexed human CD16⁺ natural killer cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of cetuximab (failed to highly activate human CD16⁺ natural killer cells to destroy cetuximab-resistant HT-29 tumor cells), complexed cetuximab is able to highly activate cetuximab-complexed human CD16⁺ natural killer cells to destroy both of HCC827-luc tumor cells and cetuximab-resistant HT-29 tumor cells through mechanisms such as antibody-dependent cell cytotoxicity (ADCC).

### Embodiment 2-1-3 Activation Marker and Cytotoxic Molecule Expression of the Cetuximab-Complexed Human CD16⁺ Natural Killer Cells after Co-Culture with Cetuximab-Responsive or Cetuximab-Resistant Human Tongue Cancer Cells

The experimental method of this embodiment is almost the same as that of Embodiment 2-1-1, except that:
(1) the effector cell used in this Experiment were \textcircled{1} the cells in the 37-day cultured oNK cell suspension, or (2) the cells in the cetuximab-complexed human CD16⁺ natural killer cell suspension; and
(2) the target cells used in this Experiment were \textcircled{1} human tongue squamous carcinoma cell line HSC-4 (JCRB0264, purchased from JCRB) that is not resistant to cetuximab, or \textcircled{2} cetuximab-resistant human tongue squamous carcinoma cell line SAS (JCRB0260, purchased from JCRB).

inventors of the present invention expect that:
(1) Whether co-cultured with HSC-4 cancer cells or SAS cancer cells, human CD16; natural killer cells (Ctrl-oNK) express only a small amount of TNF alpha, IFN gamma, granzyme B and CD107a.
(2) When co-cultured with HSC-4 cancer cells with addition of cetuximab, human CD16⁺ natural killer cells (Ctrl-oNK) would express more TNF alpha, IFN gamma, granzyme B and CD107a. However, when co-cultured with SAS cancer cells with addition of cetuximab, human CD16⁺ natural killer cells (Ctrl-oNK) would not express as many TNF alpha, IFN gamma, granzyme B and CD107a as when co-cultured with HSC-4 cancer cells with addition of cetuximab. This result indicates that in the case of HSC-4 cells, addition of cetuximab is able to highly activate human CD16⁺ natural killer cells (Ctrl-oNK) to destroy HSC-4 tumor cells through mechanisms such as antibody-dependent cell cytotoxicity (ADCC); however, in the case of SAS cells, addition of cetuximab failed to highly activate human CD16⁺ natural killer cells (Ctrl-oNK) to destroy cetuximab-resistant SAS tumor cells through mechanisms such as antibody-dependent cell cytotoxicity (ADCC).
(3) Unexpectedly, whether it was co-cultured with HSC-4 or SAS cancer cells, cetuximab-complexed human CD16⁺ natural killer cells express large amounts of TNF alpha, IFN gamma, granzyme B and CD107a, and the expression levels of various molecules such as TNF-g, IFN-γ, granzyme B and CD107a are similar in both groups. A significant enhancement of cytotoxicity of cetuximab-complexed human CD16⁺ natural killer cells against both HSC-4 and SAS compared to activation of human CD16″ natural killer cells (Ctrl-oNK) through equivalent amount of cetuximab was observed. These results indicate that as compare with co-cultured of cetuximab (failed to highly activate human CD16⁺ natural killer cells to destroy cetuximab-resistant SAS tumor cells), complexed cetuximab is able to highly activate cetuximab-complexed human CD16⁺ natural killer cells to destroy both of HSC-4 tumor cells and cetuximab-resistant SAS tumor cells through mechanisms such as antibody-dependent cell cytotoxicity (ADCC).

### Embodiment 2-2: Activation Marker and Cytotoxic Molecule Expression of the Ingredient-Complexed Human Gamma Delta T Cells after Co-Culture with Ingredient-Responsive or Ingredient-Resistant Target Cells

### Embodiment 2-2-1 Activation Marker and Cytotoxic Molecule Expression of the Trastuzumab-Complexed Human Gamma Delta T Cells after Co-Culture with Trastuzumab-Responsive or Trastuzumab-Resistant Human Breast Cancer Cells

The experimental method of this embodiment is almost the same as that of Embodiment 2-1-1, except that the effector cell used in this Experiment were \textcircled{1} the cells in the 16-day gamma delta T cell suspension, or \textcircled{2} the cells in the Trastuzumab-complexed human gamma delta T cell suspension.

inventors of the present invention expect that:
(1) Whether co-cultured with BT-474 cancer cells or BT-474 clone 5 cancer cells, human gamma delta T cells express only a small amount of TNF-α, IFN-γ, granzyme B and CD107a.
(2) When co-cultured with BT-474 cancer cells with addition of Trastuzumab, human gamma delta T cells would express more TNF-α, IFN-γ, granzyme B and CD107a. However, when co-cultured with BT-474 clone 5 cancer cells with addition of Trastuzumab, human gamma delta T cells would not express as many TNF alpha, IFN gamma, granzyme B and CD107a as when co-cultured with BT-474 cancer cells with addition of Trastuzumab. This result indicates that in the case of BT-474 cells, addition of Trastuzumab is able to highly activate human gamma delta T cells to destroy BT-474 tumor cells through mechanisms such as antibody-dependent cell cytotoxicity (ADCC); however, in the case of BT-474 clone 5 cells, addition of Trastuzumab failed to highly activate human gamma delta T cells to destroy Trastuzumab-resistant BT-474 clone 5 tumor cells through mechanisms such as antibody-dependent cell cytotoxicity (ADCC).
(3) Unexpectedly, whether it was co-cultured with BT-474 or BT-474 clone 5 cancer cells. Trastuzumab-complexed human gamma delta T cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of Trastuzumab (failed to highly activate human gamma delta T cells to destroy Trastuzumab-resistant BT-474 clone 5 tumor cells), complexed Trastuzumab is able to highly activate Trastuzumab-complexed human gamma delta T cells to destroy both of BT-474 tumor cells and Trastuzumab-resistant BT-474 clone 5 tumor cells through mechanisms such as antibody-dependent cell cytotoxicity (ADCC).

### Embodiment 2-2-2 Activation Marker and Cytotoxic Molecule Expression of the Cetuximab-Complexed Human Gamma Delta T Cells after Co-Culture with Cetuximab-Responsive Lung Cancer Cells or Cetuximab-Resistant Human Colon Cancer Cells

The experimental method of this embodiment is almost the same as that of Embodiment 2-1-2, except that the effector cell used in this Experiment were \textcircled{1} the cells in the 16-day gamma delta T cell suspension, or \textcircled{2} the cells in the cetuximab-complexed human gamma delta T cell suspension.

inventors of the present invention expect that:
(1) Whether co-cultured with HCC827-luc cancer cells or HT-29 cancer cells, human gamma delta T cells express only a small amount of TNF-α, IFN-γ, granzyme B and CD107a.
(2) When co-cultured with HCC827-luc cancer cells with addition of cetuximab, human gamma delta T cells would express more TNF-α, IFN-γ, granzyme B and CD107a. However, when co-cultured with HT-29 cancer cells with addition of cetuximab, human gamma delta T cells would not express as many TNF-α, IFN-γ, granzyme B and CD107a as when co-cultured with HCC827-luc cancer cells with addition of cetuximab. This result indicates that in the case of HCC827-luc cells, addition of cetuximab is able to highly activate human gamma delta T cells to destroy HCC827-luc tumor cells through mechanisms such as antibody-dependent cell cytotoxicity (ADCC); however, in the case of HT-29 cells, addition of cetuximab failed to highly activate human gamma delta T cells to destroy cetuximab-resistant HT-29 tumor cells through mechanisms such as antibody-dependent cell cytotoxicity (ADCC).
(3) Unexpectedly, whether it was co-cultured with HCC827-luc or HT-29 cancer cells, cetuximab-complexed human gamma delta T cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of cetuximab (failed to highly activate human gamma delta T cells to destroy cetuximab-resistant HT-29 tumor cells), complexed cetuximab is able to highly activate cetuximab-complexed human gamma delta T cells to destroy both of NCI-H508 tumor cells and cetuximab-resistant HT-29 tumor cells through mechanisms such as antibody-dependent cell cytotoxicity (ADCC).

### Embodiment 2-2-3 Activation Marker and Cytotoxic Molecule Expression of the Cetuximab-Complexed Human Gamma Delta T Cells after Co-Culture with Cetuximab-Responsive or Cetuximab-Resistant Human Tongue Cancer Cells

The experimental method of this embodiment is almost the same as that of Embodiment 2-1-1, except that:
(1) the effector cell used in this Experiment were \textcircled{1} the cells in the 16-day gamma delta T cell suspension, or \textcircled{2} the cells in the cetuximab-complexed human gamma delta T cell suspension; and
(2) the target cells used in this Experiment were \textcircled{1} human tongue squamous carcinoma cell line HSC-4 (JCRB0264, purchased from JCRB) that is not resistant to cetuximab, or \textcircled{2} cetuximab-resistant human tongue squamous carcinoma cell line SAS (JCRB0260, purchased from JCRB).

inventors of the present invention expect that:
(1) Whether co-cultured with HSC-4 cancer cells or SAS cancer cells, human gamma delta T cells express only a small amount of TNF-α, IFN-γ, granzyme B and CD107a.
(2) When co-cultured with HSC-4 cancer cells with addition of cetuximab, human gamma delta T cells would express more TNF-α, IFN-γ, granzyme B and CD107a. However, when co-cultured with SAS cancer cells with addition of cetuximab, human gamma delta T cells would not express as many TNF-α, IFN-γ, granzyme B and CD107a as when co-cultured with HSC-4 cancer cells with addition of cetuximab. This result indicates that in the case of HSC-4 cells, addition of cetuximab is able to highly activate human gamma delta T cells to destroy HSC-4 tumor cells through mechanisms such as antibody-dependent cell cytotoxicity (ADCC); however, in the case of SAS cells, addition of cetuximab failed to highly activate human gamma delta T cells to destroy cetuximab-resistant SAS tumor cells through mechanisms such as antibody-dependent cell cytotoxicity (ADCC).
(3) Unexpectedly, whether it was co-cultured with HSC-4 or SAS cancer cells, cetuximab-complexed human gamma delta T cells express large amounts of TNF alpha, IFN gamma, granzyme B and CD107a, and the expression levels of various molecules such as TNF-α, IFN-γ, granzyme B and CD107a are similar in both groups. These results indicate that as compare with co-cultured of cetuximab (failed to highly activate human gamma delta T cells to destroy cetuximab-resistant SAS tumor cells), complexed cetuximab is able to highly activate cetuximab-complexed human gamma delta T cells to destroy both of HSC-4 tumor cells and cetuximab-resistant SAS tumor cells through mechanisms such as antibody-dependent cell cytotoxicity (ADCC).

### Embodiment 2-2-4 Activation Marker and Cytotoxic Molecule Expression of the Rituximab-Complexed Human Gamma Delta T Cells after Co-Culture with Rituximab-Responsive or Rituximab-Resistant Human Lymphoma Cells

The experimental method of this embodiment is almost the same as that of Embodiment 2-1-1, except that:
(1) the effector cell used in this Experiment were \textcircled{1} the cells in the 16-day gamma delta T cell suspension, or \textcircled{2} the cells in the rituximab-complexed human gamma delta T cell suspension; and
(2) the target cells used in this Experiment were \textcircled{1} suspended human Burkitts lymphoma cell line Raji (CCL-86, purchased from ATCC) that is not resistant to rituximab, or \textcircled{2} rituximab-resistant human lymphoma cell line Raji-2R80 (or Raji-2RH; inventors of the present invention established this rituximab-resistant clones Raji-2R80 or Raji-2RH from Raji; the method of developing the rituximab-resistant clones are known, or will be apparent, to those skilled in the art, such as develop according to reference (1) Czuczman et al. Clin. Cancer Res. (2008): 14:1561-1570 (10.1158/1078-0432.CCR-07-1254); or (2) Jazirehi et al. Cancer Res. (2007): 67:1270-1281 (10.1158/0008-5472.CAN-06-2184).

inventors of the present invention expect that:
(1) Whether co-cultured with Raji cancer cells, Raji-2R80 cancer cells, or Raji-2RH cancer cells, human gamma delta T cells express only a small amount of TNF alpha, IFN gamma, granzyme B and CD107a.
(2) When co-cultured with Raji cancer cells with addition of rituximab, human gamma delta T cells would express more TNF-α, IFN-γ, granzyme B and CD107a. However, when co-cultured with Raji-2R80 (or Raji-2RH) cancer cells with addition of rituximab, human gamma delta T cells would not express as many TNF-α, IFN-γ, granzyme B and CD107a as when co-cultured with Raji cancer cells with addition of rituximab. This result indicates that in the case of Raji cells, addition of rituximab is able to highly activate human gamma delta T cells to destroy Raji tumor cells through mechanisms such as antibody-dependent cell cytotoxicity (ADCC); however, in the case of Raji-2R80 (or Raji-2RH) cells, addition of rituximab failed to highly activate human gamma delta T cells to destroy rituximab-resistant Raji-2R80 (or Raji-2RH) tumor cells through mechanisms such as antibody-dependent cell cytotoxicity (ADCC).
(3) Unexpectedly, whether it was co-cultured with Raji, Raji-2R80 or Raji-2RH cancer cells, rituximab-complexed human gamma delta T cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of rituximab (failed to highly activate human gamma delta T cells to destroy rituximab-resistant Raji-2R80 tumor cells), complexed rituximab is able to highly activate rituximab-complexed human gamma delta T cells to destroy both of Raji tumor cells and rituximab-resistant Raji-2R80 (or Raji-2RH) tumor cells through mechanisms such as antibody-dependent cell cytotoxicity (ADCC).

### Embodiment 2-2-5 Activation Marker and Cytotoxic Molecule Expression of the Rituximab-Complexed Human Gamma Delta T Cells after Co-Culture with Rituximab-Responsive or Rituximab-Resistant Human Lymphoma Cells

The experimental method of this embodiment is almost the same as that of Embodiment 2-1-1, except that:
(1) the effector cell used in this Experiment were \textcircled{1} the cells in the 16-day gamma delta T cell suspension, or \textcircled{2} the cells in the rituximab-complexed human gamma delta T cell suspension; and
(2) the target cells used in this Experiment were \textcircled{1} human lymphoma cell line Daudi (CCL-213, purchased from ATCC) that is not resistant to rituximab, or \textcircled{2} rituximab-resistant human lymphoma cell line Daudi-RR (inventors of the present invention established this rituximab-resistant clones Daudi-RR from Daudi; the method of developing the rituximab-resistant clones are known, or will be apparent, to those skilled in the art, such as develop according to reference Jazirehi et al. Cancer Res. (2007); 67:1270-1281 (10.1158/0008-5472.CAN-06-2184).

inventors of the present invention expect that:
(1) Whether co-cultured with Daudi cancer cells or Daudi-RR cancer cells, human gamma delta T cells express only a small amount of TNF-α, IFN-γ, granzyme B and CD107a.
(2) When co-cultured with Daudi cancer cells with addition of rituximab, human gamma delta T cells would express more TNF-α, IFN-γ, granzyme B and CD107a. However, when co-cultured with Daudi-RR cancer cells with addition of rituximab, human gamma delta T cells would not express as many TNF-α, IFN-γ, granzyme B and CD107a as when co-cultured with Daudi cancer cells with addition of rituximab. This result indicates that in the case of Daudi cells, addition of rituximab is able to highly activate human gamma delta T cells to destroy Daudi tumor cells through mechanisms such as antibody-dependent cell cytotoxicity (ADCC); however, in the case of Daudi-RR cells, addition of rituximab failed to highly activate human gamma delta T cells to destroy rituximab-resistant Daudi-RR tumor cells through mechanisms such as antibody-dependent cell cytotoxicity (ADCC).
(3) Unexpectedly, whether it was co-cultured with Daudi or Daudi-RR cancer cells, rituximab-complexed human gamma delta T cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of rituximab (failed to highly activate human gamma delta T cells to destroy rituximab-resistant Daudi-RR tumor cells), complexed rituximab is able to highly activate rituximab-complexed human gamma delta T cells to destroy both of Daudi tumor cells and rituximab-resistant Daudi-RR tumor cells through mechanisms such as antibody-dependent cell cytotoxicity (ADCC).

### Embodiment 2-2-6 Activation Marker and Cytotoxic Molecule Expression of the Avelumab-Complexed Human Gamma Delta T Cells after Co-Culture with Avelumab-Responsive or Avelumab-Resistant Human Breast Cancer Cells

The experimental method of this embodiment is almost the same as that of Embodiment 2-1-1, except that:
(1) the effector cell used in this Experiment were \textcircled{1} the cells in the 16-day gamma delta T cell suspension, or \textcircled{2} the cells in the avelumab-complexed human gamma delta T cell suspension; and
(2) the target cells used in this Experiment were \textcircled{1} human breast cancer cell line MDA-MB-231 (HTB-26, purchased from ATCC) that is not resistant to avelumab, or \textcircled{2} avelumab-resistant human breast cancer cell line MDA-MB-468 (HTB-132, purchased from ATCC).

inventors of the present invention expect that:
(1) Whether co-cultured with MDA-MB-231 cancer cells or MDA-MB-468 cancer cells, human gamma delta T cells express only a small amount of TNF alpha, IFN gamma, granzyme B and CD107a.
(2) When co-cultured with MDA-MB-231 cancer cells with addition of avelumab, human gamma delta T cells would express more TNF-α, IFN-γ, granzyme B and CD107a. However, when co-cultured with MDA-MB-468 cancer cells with addition of avelumab, human gamma delta T cells would not express as many TNF-α, IFN-γ, granzyme B and CD107a as when co-cultured with MDA-MB-231 cancer cells with addition of avelumab. This result indicates that in the case of MDA-MB-231 cells, addition of avelumab is able to highly activate human gamma delta T cells to destroy MDA-MB-231 tumor cells through mechanisms such as antibody-dependent cell cytotoxicity (ADCC); however, in the case of MDA-MB-468 cells, addition of avelumab failed to highly activate human gamma delta T cells to destroy avelumab-resistant MDA-MB-468 tumor cells through mechanisms such as antibody-dependent cell cytotoxicity (ADCC).
(3) Unexpectedly, whether it was co-cultured with MDA-MB-231 or MDA-MB-468 cancer cells, avelumab-complexed human gamma delta T cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of avelumab (failed to highly activate human gamma delta T cells to destroy avelumab-resistant MDA-MB-468 tumor cells), complexed avelumab is able to highly activate avelumab-complexed human gamma delta T cells to destroy both of MDA-MB-231 tumor cells and avelumab-resistant MDA-MB-468 tumor cells through mechanisms such as antibody-dependent cell cytotoxicity (ADCC).

### Embodiment 2-2-7 Activation Marker and Cytotoxic Molecule Expression of the Avelumab-Complexed Human Gamma Delta T Cells after Co-Culture with Avelumab-Responsive or Avelumab-Resistant Human Lung Cancer Cells

The experimental method of this embodiment is almost the same as that of Embodiment 2-1-1, except that:
(1) the effector cell used in this Experiment were \textcircled{1} the cells in the 16-day gamma delta T cell suspension, or \textcircled{2} the cells in the avelumab-complexed human gamma delta T cell suspension; and
(2) the target cells used in this Experiment were \textcircled{1} human lung cancer cell line H1650 (CRL-5883, purchased from ATCC) that is not resistant to avelumab, or \textcircled{2} avelumab-resistant human lung cancer cell line H2087 (CRL-5922, purchased from ATCC).

inventors of the present invention expect that:
(1) Whether co-cultured with H1650 cancer cells or H2087 cancer cells, human gamma delta T cells express only a small amount of TNF alpha, IFN gamma, granzyme B and CD107a.
(2) When co-cultured with H1650 cancer cells with addition of avelumab, human gamma delta T cells would express more TNF-α, IFN-γ, granzyme B and CD107a. However, when co-cultured with H2087 cancer cells with addition of avelumab, human gamma delta T cells would not express as many TNF-α, IFN-γ, granzyme B and CD107a as when co-cultured with H1650 cancer cells with addition of avelumab. This result indicates that in the case of H1650 cells, addition of avelumab is able to highly activate human gamma delta T cells to destroy H1650 tumor cells through mechanisms such as antibody-dependent cell cytotoxicity (ADCC); however, in the case of H2087 cells, addition of avelumab failed to highly activate human gamma delta T cells to destroy avelumab-resistant H2087 tumor cells through mechanisms such as antibody-dependent cell cytotoxicity (ADCC).
(3) Unexpectedly, whether it was co-cultured with H1650 or H2087 cancer cells, avelumab-complexed human gamma delta T cells express large amounts of TNF-α, IFN-γ, granzyme B and CD107a, and the expression levels of various molecules such as TNF-α, IFN-γ, granzyme B and CD107a are similar in both groups. These results indicate that as compare with co-cultured of avelumab (failed to highly activate human gamma delta T cells to destroy avelumab-resistant H2087 tumor cells), complexed avelumab is able to highly activate avelumab-complexed human gamma delta T cells to destroy both of H1650 tumor cells and avelumab-resistant H2087 tumor cells through mechanisms such as antibody-dependent cell cytotoxicity (ADCC).

### Embodiment 3: The Effect of Immunosuppressive Microenvironment on the Cytotoxic Function of Ingredient-Complexed Human Cytotoxic Cells

### Embodiment 3-1: The Effect of Hypoxia on the Cytotoxic Function of Ingredient-Complexed Human Cytotoxic Cells

Lactate dehydrogenase (LDH) cytotoxicity assay kit (Cat No, 88954, Pierce Biotechnology, USA) was used in this embodiment to detect the cytotoxic ability of the effector cells toward target cells. This embodiment comprised a 96 well culture plate to carry out cytotoxicity test, and the wells were divided into Effector cell spontaneous LDH release control wells (ESR, including ACE1702 spontaneous LDH release control wells and Ctrl-oNK spontaneous LDH release control wells). Target cell spontaneous LDH release control wells (TSR). Experimental wells (including ACE1702 Experimental wells and Ctrl-oNK Experimental wells). Target cell maximum LDH release control well (TMR), Volume correction control well (VCC, this well is for correcting the volume increase caused by addition of lysis buffer), and Culture medium background control well (CMB, this well is for correcting the contributions caused by LDH activity that may be present in serum-containing culture medium).

The effector cells used in this embodiment were \textcircled{1} the cells in the 26-day cultured oNK cell suspension (Ctrl-oNK), or \textcircled{2} the cells in the Trastuzumab-complexed human CD16⁺ natural killer cell suspension (ACE1702), and the target cells were an adherent human ovarian cancer cell line SK-OV-3 (HTB-77, purchased from ATCC).

Target cells were seeded in Target cell spontaneous LDH release control wells (TSR), Experimental wells, and Target cell maximum LDH release control well (TMR); hence, each well contained 1.2×10⁴ target cells, and were allowed to sit 2-4 hours.

Effector cells (ACE1702 or Ctrl-oNK) and target cells (SK-OV-3) were pretreated with hypoxic condition (0% oxygen condition) for 18 hours at 37° C. Pre-treated Effector cells (ACE1702 or Ctrl-oNK) was added to the Effector cell spontaneous LDH release control wells (ESR) and experimental wells, and the ratio of the number of effector cell to the number of target cells was 5. The same volume of serum-containing culture medium was added into the Volume correction control well (VCC) and Culture medium background control well (CMB).

The effector cells and target cells were co-cultured in the 96 well culture plate with hypoxia condition for 18 hours, and 10 μL lysis buffer was added into Target cell maximum LDH release control well (TMR) and Volume correction control well (VCC) before harvesting the supernatant.

The 96 well culture plate was centrifuged at room temperature at 400×g for 3 min, then 50 μL supernatant was transferred from each well to the new wells of a different new 96-well plate respectively. The wells were observed under the microscope to inspect if any cell was present, 50 μL Reaction Mixture (containing a substrate “tetrazolium salt” of Lactate dehydrogenase; commercially available from Pierce Biotechnology: Lactate dehydrogenase cytotoxicity assay kit. Cat No, 88954) was added to each of the new wells and mix gently, then incubated at room temperature for 5-15 minutes. The Stop Solution was added to each of the new wells and the absorbance at 490 nm and 680 nm was measured.

As the greater the OD₄₉₀-OD₆₈₀, the higher the Lactate dehydrogenase released from lysed target cells. OD₄₉₀-OD₆₈₀ can be used to convert the percentage of target cells that are lysed in the Experimental well. Please refer to the formula described in the Lactate dehydrogenase (LDH) cytotoxicity assay kit (Cat No, 88954, Pierce Biotechnology. USA) to convert the OD₄₉₀-OD₆₈₀ to the percentage of target cells that are lysed in the Experimental well.

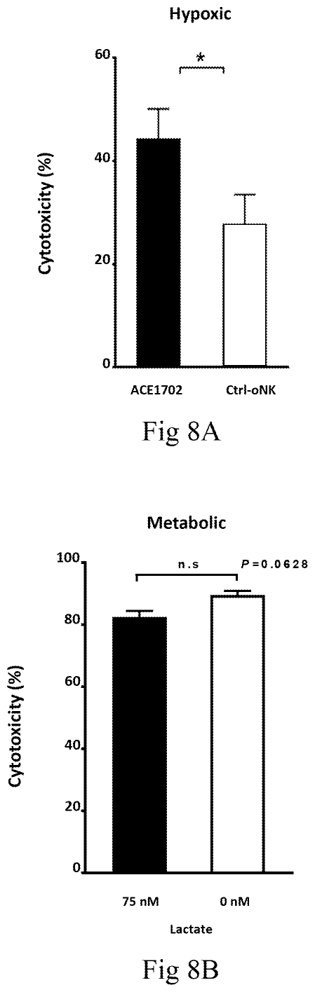

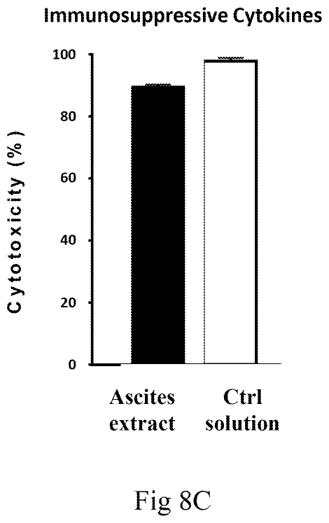

Please refer to FIG. 8A. FIG. 8A is a bar chart presenting the effect of hypoxia on the cytotoxic function of ingredient-complexed cytotoxic cells and the non-complexed human cytotoxic cells. Cells in the Trastuzumab-complexed human CD16⁺ natural killer cell suspension (ACE1702) were pre-treated in hypoxic condition and then cocultured with ovarian cancer cell line SK-OV-3 cells with hypoxic condition at E/T ratio 5 at 37° C. for 18 hours, and the lysis percentage of SK-OV-3 cells was 43%. With regards to the cytotoxicity of Ctrl-oNK, the percentage of SK-OV-3 lysis by the cells in the 24-day cultured oNK cell suspension (Ctrl-oNK) was 25%. The result indicates that ACE1702 cells maintained enhanced cytotoxicity against human ovarian cancer cell line SK-OV-3 cells compared to Ctrl-oNK cells in immunosuppressive microenvironment such as hypoxic conditions.

### Embodiment 3-2: The Effect of Lactate (a Metabolic Waste) on the Cytotoxic Function of Ingredient-Complexed Human Cytotoxic Cells

The experimental method of this embodiment is almost the same as that of Embodiment 3-1, except that:
(1) The effector cells used in this Experiment were the cells in the Trastuzumab-complexed human CD16⁺ natural killer cell suspension; and
(2) The effector cells were pretreated with 0 or 75 mM lactate for 1 hour at room temperature instead of pretreated with hypoxic condition (0% oxygen condition) for 18 hours at 37° C.
(3) The experimental wells include 0 mM lactate Experimental wells and 75 mM lactate Experimental wells.
(4) The effector cells and target cells in the 96 well culture plate were co-cultured for 15 hours.

Please refer to FIG. 8B. FIG. 8B is a bar chart presenting the effect of lactate (a metabolic waste) on the cytotoxic function of ingredient-complexed cytotoxic cells. Cells in the Trastuzumab-complexed human CD16⁺ natural killer cell suspension (ACE1702) were pre-treated with 75 mM lactate and then co-cultured with ovarian cancer cell line SK-OV-3 cells at E/T ratio 5 at 37° C. for 15 hours, and the lysis percentage of SK-OV-3 cells was 80%. With regard to the cytotoxicity of ACE1702 pre-treated with 0 mM lactate, the lysis percentage of SK-OV-3 cells was 90% (p>0.05). The result indicates that ACE1702 cells maintained high cytotoxicity against human ovarian cancer cell line SK-OV-3 cells in immunosuppressive microenvironment such as acidic conditions.

### Embodiment 3-3: The Effect of Ascites Extract (Comprising Immunosuppressive Cytokines) on the Cytotoxic Function of Ingredient-Complexed Human Cytotoxic Cells

The experimental method of this embodiment is almost the same as that of Embodiment 3-1, except that:
(1) The effector cells used in this Experiment were the cells in the Trastuzumab-complexed human CD16⁺ natural killer cell suspension (ACE1702); and
(2) The effector cells were not pre-treated with hypoxic condition (0% oxygen condition) for 18 hours at 37° C.
(3) Experimental wells include 0% ascites extract Experimental wells and 50% ascites extract Experimental wells.
(4) The effector cells and target cells in the 96 well culture plate were co-cultured at E:T ratio of 10 in oNK growth medium in the absence or presence of 50% (v/v %) of ascites extract for 18 hours. Wherein the ascites extract was obtained based on the following method:

Abdominal Paracentesis was performed by a medical doctor. In brief, a paracentesis needle was inserted slowly into the abdomen of a human subject with ovarian cancer to abstract the abdomen fluid from the human subject. The abstracted abdomen fluid was centrifuged at 1500×g for 5 minutes to remove free-floating cells. The supernatant was collected as ascites extract. The ascites extract was examined by microscopy or flow cytometry to make sure that it is cell-free.

Please refer to FIG. 8C. FIG. 8C is a bar chart presenting the effect of ascites extract (comprising immunosuppressive cytokines) on the cytotoxic function of ingredient-complexed human cytotoxic cells. Cells in the Trastuzumab-complexed human CD16⁺ natural killer cell suspension (ACE1702) were cocultured with ovarian cancer cell line SK-OV-3 cells at E/T ratio 5 at 37° C. for 18 hours in the presence of 50% (v/v %) of ascites extract, and the lysis percentage of SK-OV-3 cells was 90%. With regards to the cytotoxicity of ACE1702 cultured in the absence of ascites extract, the lysis percentage of SK-OV-3 cells was 95% (p>0.05). The result indicates that ACE1702 cells maintained high cytotoxicity against human ovarian cancer cell line SK-OV-3 cells in immunosuppressive microenvironment such as comprising immunosuppressive cytokines.

### Embodiment 4: Cytotoxicity of Ingredient-Complexed Cytotoxic Cells Against Ingredient-Resistant Solid Tumor in a Subject

### Embodiment 4-1: Cytotoxicity of Ingredient-Complexed Human CD16⁺ Natural Killer Cells Against Ingredient-Resistant Solid Tumor in a Subject

Luciferase-expressing trastuzumab-resistant breast cancer cell line BT474 Clone 5 (CRL-3247, purchased from ATCC) was intraperitoneally injected into each of the 25 female NOG mice (Jackson Laboratory) on Day 0. The mice were divided into 5 groups randomly.
(1) Mice in the Ctrl-oNK group were treated with 5000000 cells in the 24-day cultured oNK cell suspension on Day 0, 3, 7, 10, 14, and 17.
(2) Mice in the ACE1702 group were treated with 5000000 cells in the trastuzumab-complexed human CD16⁺ natural killer cell suspension on Day 0, 3, 7, 10, 14, and 17.
(3) Mice in the Ctrl-oNK and Trastuzumab group were treated with 5000000 cells in the 24-day cultured oNK cell suspension and 2.75 ng of trastuzumab (an antibody against HER2 protein with product name as Herceptin was purchased from Roche, Swiss) on Day 0, 3, 7, 10, 14, and 17. Therefore, the amount of trastuzumab administered into the mice in the Ctrl-oNK and trastuzumab group (2.75 ng of trastuzumab) was same as the total amount of the trastuzumab linked to the cells that were administered into the mice in the ACE1702 group.
(4) Mice in the trastuzumab group were treated with 2.75 ng of trastuzumab on Day 0, 3, 7, 10, 14, and 17.
(5) Mice in the control group were treated with Vehicle (cell medium only, such as cell culture medium, DMEM culture medium or XVIVO 10 culture medium described in Experiment 1-1-1) on Day 0, 3, 7, 10, 14, and 17.

Luminescence was detected by AMI HTX (Spectral Imaging) on Day 0, 3, 7, 10, 14 and 17 and weekly after Day 17 until the end of the experiment.

Inventor of the present invention expect:

The fluorescent images of mice show that: bioluminescence image of mice treated with cells in the trastuzumab-complexed human CD16⁺ natural killer cell suspension demonstrate significant reduction. Therefore, the ingredient-complexed cytotoxic cells of the present invention could treat ingredient-resistant solid tumor in a subject as well as treating abnormal cells located in the immunosuppressive microenvironment such as solid tumor.

To further understand the mechanism of the ingredient-complexed cytotoxic cells of the present invention reducing the abnormal cells located in the immunosuppressive microenvironment such as solid tumor, inventor of the present invention proceed cell migration studies of Embodiment 5 and Embodiment 6.

### Embodiment 4-2 Cytotoxicity of Ingredient-Complexed Human Gamma Delta T Cells Against Ingredient-Resistant Solid Tumor in a Subject

Luciferase-expressing trastuzumab-resistant breast cancer cell line BT474 Clone 5 (CRL-3247, purchased from ATCC) was intraperitoneally injected into each of the 25 female NOG mice (Jackson Laboratory) on Day 0. The mice were divided into 5 groups randomly.
(1) Mice in the Ctrl-gdT group were treated with 10000000 cells in the 16-day gamma delta T cell suspension on Day 0, 3, 7, 10, 14, and 17.
(2) Mice in the ACE-gdT-HER2 group were treated with 10000000 cells in the trastuzumab-complexed human gamma delta T cell suspension on Day 0, 3, 7, 10, 14, and 17.
(3) Mice in the Ctrl-gdT and trastuzumab group were treated with 10000000 cells in the 16-day gamma delta T cell suspension and 2.75 ng of trastuzumab (an antibody against HER2 protein with product name as Herceptin was purchased from Roche, Swiss) on Day 0, 3, 7, 10, 14 and 17. Therefore, the amount of trastuzumab administered into the mice in the Ctrl-gdT and trastuzumab group (2.75 ng of Trastuzumab) was same as the total amount of the trastuzumab linked to the cells that were administered into the mice in the ACE-gdT-HER2 group.
(4) Mice in the trastuzumab group were treated with 2.75 ng of trastuzumab on Day 0, 3, 7, 10, 14 and 17.
(5) Mice in the control group were treated with Vehicle (cell medium only, such as complete growth medium described in Experiment 1-2-1) on Day 0, 3, 7, 10, 14 and 17.

Luminescence was detected by AMI HTX (Spectral Imaging) on Day 0, 3, 7, 10, 14 and 17 and weekly after Day 17 until the end of the experiment.

Inventor of the present invention expect:

The luminescence images of mice show that: bioluminescence image of mice treated with cells in the trastuzumab-complexed human gamma delta T cell suspension demonstrate significant reduction. Therefore, the ingredient-complexed cytotoxic cells of the present invention could treat ingredient-resistant solid tumor in a subject as well as treating abnormal cells located in the immunosuppressive microenvironment such as solid tumor.

To further understand the mechanism of the ingredient-complexed cytotoxic cells of the present invention reducing the abnormal cells located in the immunosuppressive microenvironment such as solid tumor, inventor of the present invention proceed cell migration studies of Embodiment 5 and Embodiment 6.

### Embodiment 5: Ingredient-Complexed Cytotoxic Cell Migration Study

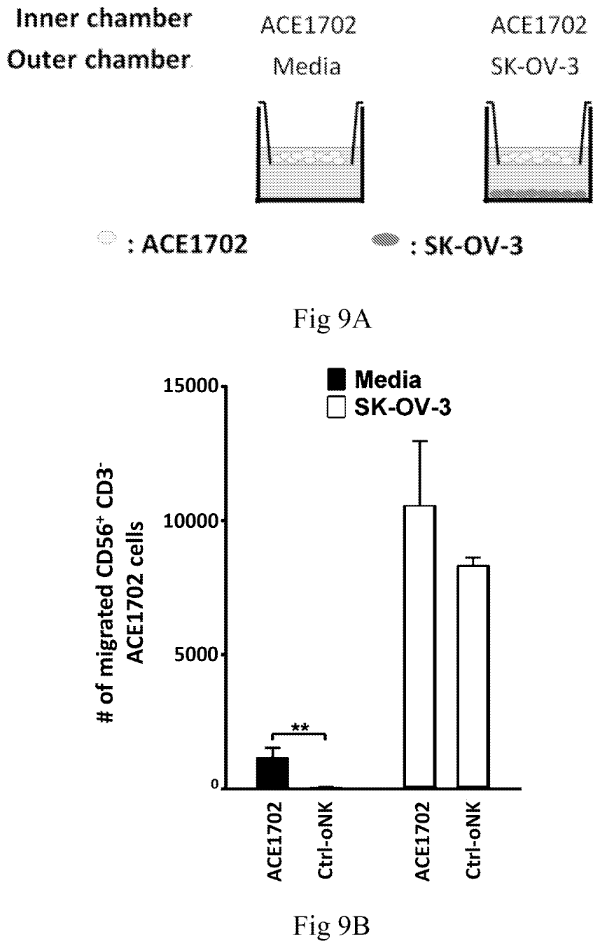

Please refer to FIG. 9A. FIG. 9A illustrates the assay design of ingredient-complexed cytotoxic cell migration study. The outer chamber used in this embodiment was a 24-well plate (ThermoScientific, Cat, 142475). Wells of the outer chamber were divided into medium wells and SK-OV-3 wells. The inner chambers used in this embodiment were polycarbonate cell culture inserts (Millipore, Cat. PITP01250). Cell medium (such as cell culture medium, DMEM culture medium or XVIVO 10 culture medium described in Experiment 1-1-1) or 2×10⁵ SK-OV-3 cells (Target cells) in Cell medium were seeded in the wells of the outer chamber. After 2 hours of attachment, polycarbonate cell culture inserts with pore size of 3 μm (Millicell cell culture insert) were inserted into each well of the outer chamber, and 1×10⁶ cells in the trastuzumab-complexed human CD16⁺ natural killer cell suspension were seeded into each inner chamber for 19 hours of incubation.

Cells in the outer chamber were harvested and stained with PE-conjugated mouse anti-human CD56 (BioLegend, #304606) and PE-Cy5-conjugated CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended CD56/CD3-gated cells was analyzed by flow cytometry.

Please refer to FIG. 9B. FIG. 9B is a bar chart presenting the migratory capacity of the ingredient-complexed cytotoxic cells into a region where cancer cells are located. FIG. 9B demonstrates only small amount of trastuzumab-complexed human CD16⁺ natural killer cells (trastuzumab-complexed oNK cells expressing CD56 and CD16) was detected in medium wells: on the other hand, a large amount of trastuzumab-complexed human CD16⁺ natural killer cells (trastuzumab-complexed oNK cells expressing CD56 and CD16) was detected in SK-OV-3 wells. Moreover, the amount of trastuzumab-complexed human CD16⁺ natural killer cell in SK-OV-3 wells is 9.1 times more than those in medium wells. That is, compared to the number of ingredient-complexed cytotoxic cells moving to the outer chamber containing medium, the number of ingredient-complexed cytotoxic cells moving through the membrane at the bottom of the polycarbonate cell culture inserts and migrate to the outer chamber containing the target cells increased significantly by 9.1 times (each condition was performed in triplicate, and the data was presented as mean±S.D. Statistical analysis was performed using t test. **, p<0.01.)

Inventors of the present invention expect that if the forementioned SK-OV-3 is replaced with target cells resistant to an ingredient (such as BT-474 clone 5 cell line, HT-29 cell line, and SAS cell line), large amount of cytotoxic cells complexed with the ingredient (ingredient-complexed cytotoxic cells) in the inner chamber can also pass through the membrane at the bottom of the polycarbonate cell culture inserts and move to the outer chamber containing the target cells resistant to an ingredient.

### Embodiment 6: CD3⁺ T Cells Migration Study

### Embodiment 6-1: Effect of the Ingredient-Complexed Human CD16⁺ Natural Killer Cells on the Migratory Capacity of the CD3⁺ T Cells

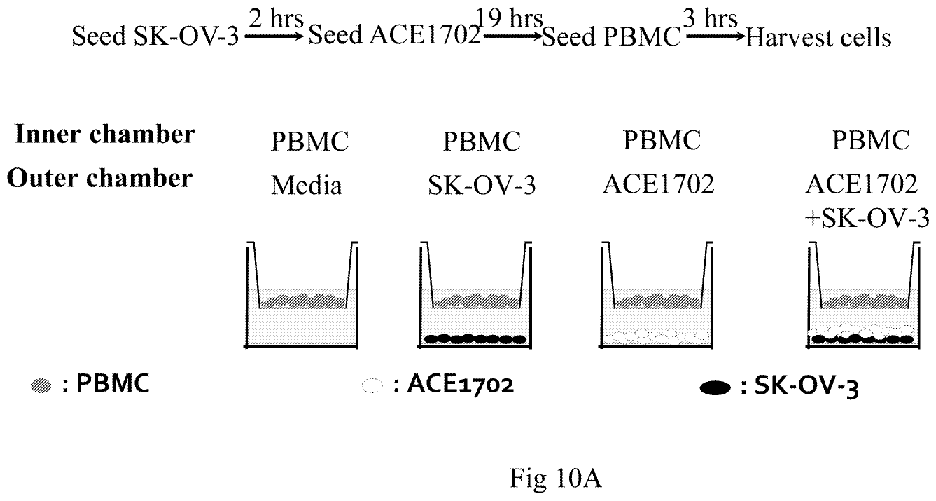

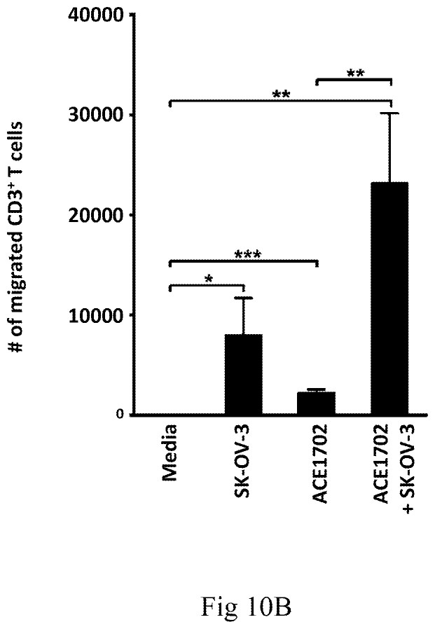

Please refer to FIG. 10A. FIG. 10A illustrates the assay design of CD3⁺ T cells migration study. The outer chamber used in this embodiment was a 24-well plate (ThermoScientific, Cat, 142475). The inner chambers used in this embodiment were polycarbonate cell culture inserts with pore size of 3 μm (Millicell cell culture insert: Cat. PITP01250).

There were four groups in this embodiment, which were (1) medium group, (2) SK-OV-3 group, (3) ACE1702 group, and (4) SK-OV-3 and ACE1702 group.
(1) Medium group: cell medium (such as cell culture medium, DMEM culture medium or XVIVO 10 culture medium described in Experiment 1-1-1) was added into the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(2) SK-OV-3 group: 4×10⁵ SK-OV-3 cells (Target cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(3) ACE1702 group: 1×10⁶ cells in the trastuzumab-complexed human CD16⁺ natural killer cell suspension were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(4) SK-OV-3 and ACE1702 group: 4×10⁵ SK-OV-3 cells (Target cells) and 1×10⁶ cells in the trastuzumab-complexed human CD16⁺ natural killer cell suspension were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.

Cells in the outer chamber were harvested and stained with CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended CD3⁺ gated cells was analyzed by flow cytometry.

Please refer to FIG. 10B. FIG. 10B is a bar chart presenting the effect of the ingredient-complexed cytotoxic cells on the migratory capacity of the CD3⁺ T cells into a region where cancer cells are located. FIG. 10B shows that the outer chamber of the medium group contains 35 CD3⁺ T cells; the outer chamber of the SK-OV-3 group contains 8031 CD3⁺ T cells; the outer chamber of the ACE1702 group contains 2257 CD3⁺ T cells; the outer chamber of SK-OV-3 and ACE1702 group contains 23183 CD3⁺ T cells. That is, compared with the number of CD3⁺ T cells moving to the outer chamber containing medium, the number of CD3⁺ T cells moving through the membrane at the bottom of the polycarbonate cell culture inserts and migrate to the outer chamber containing target cells alone, ACE1702 alone, and co-culture of ACE1702 and target cells increased significantly up to 229.5 times, 64.5 times, and 662.4 times respectively (each condition was performed in triplicate and the data was presented as mean±S.D. Statistical analysis was performed using t test. *, p<0.05; **, p<0.01; ***, p<0.001.)

These results demonstrate that the ingredient-complexed cytotoxic cells in the outer chamber (lesion simulation) that comprises target cells such as SK-OV-3 cells would significantly increase the migratory capacity of CD3⁺ T cells into the lesion.

By comparing among the CD3⁺ T cells migration efficacy of the SK-OV-3 group and the ACE1702 group, it demonstrates that the target cells (such as SK-OV-3) and ingredient-complexed cytotoxic cells in the outer chamber of the SK-OV-3 and ACE1702 group unexpectedly manifest synergy in the CD3⁺ T cells migration efficacy.

If the forementioned SK-OV-3 is replaced with target cells resistant to an ingredient (such as BT-474 clone 5 cell line. HT-29 cell line, and SAS cell line), inventors of the present invention expect that it would have results similar to the forementioned experiments, including:
(1) Cytotoxic cells complexed with the ingredient (ingredient-complexed cytotoxic cells) in the outer chamber (lesion simulation) that comprises target cells resistant to the ingredient would significantly increase the migratory capacity of CD3⁺ T cells into the lesion; and
(2) The target cells resistant to the ingredient and the ingredient-complexed cytotoxic cells in the outer chamber would unexpectedly manifest synergy in the CD3⁺ T cells migration efficacy.

### Embodiment 6-2: Effect of the Ingredient-Complexed Human Gamma Delta T Cells on the Migratory Capacity of the CD3⁺ T Cells and CD56⁺CD3-NK Cells

There were four groups in this embodiment, which were (1) medium group, (2) SK-OV-3 group, (3) ACE-gdT-HER2 group, and (4) SK-OV-3 and ACE-gdT-HER2 group.
(1) Medium group: cell medium (such as complete growth medium described in Experiment 1-2-1) was added into the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells and CD56⁺CD3-NK cells in PBMC cells.
(2) SK-OV-3 group: 4×10⁵ SK-OV-3 cells (Target cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells and CD56⁺CD3-NK cells in PBMC cells.
(3) ACE-gdT-HER2 group: 1×10⁶ cells in the trastuzumab-complexed human gamma delta T cell suspension were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells and CD56⁺CD3-NK cells in PBMC cells.
(4) SK-OV-3 and ACE-gdT-HER2 group: 4×10⁵ SK-OV-3 cells (Target cells) and 1×10⁶ cells in the trastuzumab-complexed human gamma delta T cell suspension were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells and CD56 CD3-NK cells in PBMC cells.

Cells in the outer chamber were harvested and stained with CD56 and CD3 antibodies at room temperature for 10 minutes while avoiding the light. Cell mixtures were then centrifuged at room temperature at 400×g for 3 minutes. Supernatant was removed, and cell pellet was resuspended with 1 ml of DPBS. Centrifugation was repeated, and 0.5 mL of DPBS-resuspended CD3⁺ and CD56 CD3-gated cells was analyzed by flow cytometry.

Inventors of the present invention expect that the ingredient-complexed cytotoxic cells in the outer chamber (lesion simulation) that comprises target cells such as SK-OV-3 cells would significantly increase the migratory capacity of CD3⁺ T cells and CD56 CD3-NK cells into the lesion. By comparing among the CD3⁺ T cells and CD56 CD3-NK cells migration efficacy of the SK-OV-3 group and the ACE-gdT-HER2 group, it is expected that the target cells (such as SK-OV-3) and ingredient-complexed cytotoxic cells in the outer chamber of the SK-OV-3 and ACE-gdT-HER2 group surprisingly manifest synergy in the CD3⁺ T cells and CD56 CD3-NK cells migration efficacy.

If the forementioned SK-OV-3 is replaced with target cells resistant to an ingredient (such as BT-474 clone 5 cell line. HT-29 cell line, and SAS cell line), inventors of the present invention expect that it would have results similar to the forementioned experiments, including:
(1) Cytotoxic cells complexed with the ingredient (ingredient-complexed cytotoxic cells) in the outer chamber (lesion simulation) that comprises target cells resistant to the ingredient would significantly increase the migratory capacity of CD3⁺ T cells and CD56 CD3-NK cells into the lesion; and
(2) The target cells resistant to the ingredient and the ingredient-complexed cytotoxic cells in the outer chamber would surprisingly manifest synergy in the CD3⁺ T cells and CD56⁺CD3-NK cells migration efficacy.

### Embodiment 7: Effect of the Number of Complexed Ingredients on the Function of Ingredient-Complexed Cytotoxic Cells

### Embodiment 7-1: Effect of the Number of Complexed Ingredients on the Function of the Ingredient-Complexed Human CD16⁺ Natural Killer Cells

### Embodiment 7-1-1 Preparation of Human CD16⁺ Natural Killer Cells Complexed with Different Number of Ingredient

In this experiment, the cultured oNK cell suspensions that was obtained by culturing for 24 days with the culture method disclosed in the Experiment 1-1-1 (24-day cultured oNK cell suspension of the present invention, refer to as 24-day cultured oNK cell suspension) were used to prepare human CD16⁺ natural killer cells complexed with different number of ingredient (such as trastuzumab, cetuximab, rituximab, or avelumab).

After preparing cell-ssDNA conjugate by binding different amount of cell linkers to the cells in the 24-day cultured oNK cell suspension and then mixing the cell-ssDNA conjugate and the ingredient-ssDNA according to the chemical conjugation technology described in Embodiment 1-1-4, human CD16⁺ natural killer cells complexed with different number of ingredient (such as trastuzumab, cetuximab, rituximab, or avelumab) were obtained.

The ingredient-complexed human CD16⁺ natural killer cell suspension was mixed with phycoerythrin-conjugated goat-anti-human Fab antibody (e.g., purchased form Jackson ImmunoResearch Laboratories. Inc.), so that the complexed ingredient (such as trastuzumab, cetuximab, rituximab, or avelumab) was interacted specifically with the phycoerythrin-conjugated goat-anti-human Fab antibody.

To convert mean fluorescence intensity of phycoerythrin-conjugated goat-anti-human Fab antibody into number of the ingredient (such as trastuzumab, cetuximab, rituximab, or avelumab) complexed on each of the human CD16⁺ natural killer cells, standard curves derived from Quantum™ Simply Cellular® kit (Bangs Laboratories, Inc, #815) were developed. There were 5 bottles of microspheres (+ populations “#1, #2, #3 and #1” coated with increasing amounts of anti-human IgG antibody, 1 uncoated blank) in the Quantum™ Simply Cellular® kit. Ten microliter of anti-human IgG antibody-bound microspheres, including #1, #2, #3 and #4 microsphere were individually incubated with 1 μg/mL of the ingredient (such as trastuzumab, cetuximab, rituximab, or avelumab) in total 0.1 mL reaction volume at room temperature for 30 minutes. For the blank microsphere, the similar procedure but without the addition of the ingredient (such as trastuzumab, cetuximab, rituximab, or avelumab) was performed. The trastuzumab-bound #1 to #4 microspheres and the blank microspheres were then detected by the above mentioned phycoerythrin-conjugated goat-anti-human Fab antibody. Microspheres were washed with 0.5 mL of DPBS and the suspension was centrifuged at 400×g at room temperature for 5 minutes. The supernatant was removed and the suspended QSC microspheres were analyzed by flow cytometry. Acquired mean fluorescence intensity value of each microsphere was inserted into respective columns of manufacturer-provided calculation sheet (QuickCal V2.3) to generate the corresponding standard curve following manufacturer's instruction. After individually developing the standard curve of the absolute number of each ingredient (such as trastuzumab, cetuximab, rituximab, or avelumab), 0, 1000±500, 3000±500.6000±500, 12000±3000, 20000±5000, 30000±5000, 50000±5000, or 130000±5000 ingredient molecules (such as trastuzumab molecules, cetuximab molecules, rituximab molecules, or avelumab molecules) was next inserted to the QuickCal sheet to convert into corresponding fluorescence intensity value of the phycoerythrin-conjugated goat-anti-human Fab antibody-stained human CD16^{±} natural killer cells that complexed with 0, 1000±500, 3000±500, 6000±500, 12000±3000, 20000±5000, 30000±5000, 50000±5000, or 130000±5000 ingredient molecules per cell.

The cell sorter (BD FACSMelody, BD FACSAria III, SONY SH800S, etc.) was used to isolate human CD16^{±} natural killer cells that complexed with 0, 1000±500, 3000=500, 6000±500, 12000±3000, 20000±5000, 30000±5000, 50000±5000, or 130000±5000 ingredient molecules (such as trastuzumab molecules, cetuximab molecules, rituximab molecules, or avelumab molecules) per cell.

There were nine kinds of effector cells used in the following Embodiments 7-1-2 to 7-1-5:
(1) oNK cells complexed with 0 trastuzumab molecule (Ctrl-oNK cells): cells in the 28-day cultured oNK cell suspension were used in this group.
(2) oNK cells complexed with 1000 trastuzumab molecules: human CD16⁺ natural killer cells that complexed with 500 to 1500 trastuzumab molecules per cell were used in this group.
(3) oNK cells complexed with 3000 trastuzumab molecules: human CD16⁺ natural killer cells that complexed with 2500 to 3500 trastuzumab molecules per cell were used in this group.
(4) oNK cells complexed with 6000 trastuzumab molecules: human CD16⁺ natural killer cells that complexed with 5500 to 6500 trastuzumab molecules per cell were used in this group.
(5) oNK cells complexed with 12000 trastuzumab molecules: human CD16⁺ natural killer cells that complexed with 9000 to 15000 trastuzumab molecules per cell were used in this group.
(6) oNK cells complexed with 20000 trastuzumab molecules: human CD16⁺ natural killer cells that complexed with 15000 to 25000 trastuzumab molecules per cell were used in this group.
(7) oNK cells complexed with 30000 trastuzumab molecules: human CD16⁺ natural killer cells that complexed with 25000 to 35000 trastuzumab molecules per cell were used in this group.
(8) oNK cells complexed with 50000 trastuzumab molecules: human CD16⁺ natural killer cells that complexed with 45000 to 55000 trastuzumab molecules per cell were used in this group.
(9) oNK cells complexed with 130000 trastuzumab molecules: human CD16⁺ natural killer cells that complexed with 125000 to 135000 trastuzumab molecules per cell were used in this group.

### Embodiment 7-1-2 Effect of the Number of Complexed Ingredients on the Cytotoxic Function of the Ingredient-Complexed Human CD16⁺ Natural Killer Cells

CellTiter-Glo® Luminescent Cell Viability Assay (Promega, USA) was used in this embodiment to detect the cytotoxic ability of the effector cells toward target cells. First, wells in a CELLSTAR® 96 well plates (Cat. Number 655083, purchased from Greiner) were divided into:
(1) target control well.
(2) oNK cells complexed with 0 trastuzumab molecule basal well.
(3) oNK cells complexed with 0 trastuzumab molecule and target experimental well.
(4) oNK cells complexed with 1000 trastuzumab molecule basal well.
(5) oNK cells complexed with 1000 trastuzumab molecule and target experimental well.
(6) oNK cells complexed with 3000 trastuzumab molecule basal well.
(7) oNK cells complexed with 3000 trastuzumab molecule and target cell experimental well.
(8) oNK cells complexed with 6000 trastuzumab molecule basal well, and
(9) oNK cells complexed with 6000 trastuzumab molecule and target cell experimental well.
(10) oNK cells complexed with 12000 trastuzumab molecule basal well, and
(11) oNK cells complexed with 12000 trastuzumab molecule and target cell experimental well.

The target cells used in this Experiment were \textcircled{1} human breast cancer cell line BT474 (HTB-20, purchased from ATCC), or \textcircled{2} BT474's derived trastuzumab-resistant clone BT474 Clone 5 (CRL-3247, purchased from ATCC).

Target cells (BT474 or BT474 Clone 5 cells) were seeded in:
the target control wells.
the oNK cells complexed with 0 trastuzumab molecule and target experimental wells.
the oNK cells complexed with 1000 trastuzumab molecule and target experimental wells.
the oNK cells complexed with 3000 trastuzumab molecule and target experimental wells.
the oNK cells complexed with 6000 trastuzumab molecule and target experimental wells, and
the oNK cells complexed with 12000 trastuzumab molecule and target experimental wells.
so that each well-contained 5000 target cells.

A sample of the 28-day cultured oNK cell suspension was added to:
the oNK cells complexed with 0 trastuzumab molecule basal wells, and
the oNK cells complexed with 0 trastuzumab molecule and target experimental wells.

A sample of the human CD16⁺ natural killer cells that complexed with 500 to 1500 trastuzumab molecules per cell was added to:
the oNK cells complexed with 1000 trastuzumab molecule basal wells, and
the oNK cells complexed with 1000 trastuzumab molecule and target experimental wells.

A sample of the human CD16⁺ natural killer cells that complexed with 2500 to 3500 trastuzumab molecules per cell was added to:
the oNK cells complexed with 3000 trastuzumab molecule basal wells, and
the oNK cells complexed with 3000 trastuzumab molecule and target experimental wells.

A sample of the human CD16⁺ natural killer cells that complexed with 5500 to 6500 trastuzumab molecules per cell was added to:
the oNK cells complexed with 6000 trastuzumab molecule basal wells, and
the oNK cells complexed with 6000 trastuzumab molecule and target experimental wells.

A sample of the human CD16⁺ natural killer cells that complexed with 9000 to 15000 trastuzumab molecules per cell was added to:
the oNK cells complexed with 12000 trastuzumab molecule basal wells, and
the oNK cells complexed with 12000 trastuzumab molecule and target experimental wells.

The ratio of the number of effector cell to the number of target cell (BT474 or BT474 Clone 5 cells) in these wells was the same in each group.

The CELLSTAR® 96 well plates were incubated at 37° C. in 5% CO₂ for 4 hours. After 4 hours of incubation, the culture were mixed with 50 μL of CellTiter® Glo substrate (provided in the CellTiter-Glo® luminescent cell viability assay kit. Promega. Cat. G7570) and incubated at room temperature without light for 12 minutes. The luminescence of each well was measured and recorded by luminescence plate reader (Synergy H1. BioTek Instruments, USA).

Wherein, the greater the number of live cells remained in the well, the higher the luminescence detected by the Synergy H1 system. Therefore, the luminescence can be used to convert the percentage of target cells that are lysed in the experimental well. The formula used to convert the luminescence to the percentage of target cells that are lysed in the experimental wells are as follows:

$$Percentage of lysed target cell in “ oNK cells complexed with 0 trastuzumab molecule and target experimental well ” ( \% ) = 1 - [ ( luminescence of oNK cells complexed with 0 trastuzumab molecule and target experimental well - luminescence of oNK cells complexed with 0 trastuzumab molecule basal well ) \div ( luminescence of target control well ) ] \times 100 \%$$

$$Percentage of lysed target cell in “ oNK cells complexed with 1000 trastuzumab molecule and target experimental well ” ( \% ) = 1 - [ ( luminescence of oNK cells complexed with 1000 trastuzumab molecule and target experimental well - luminescence of oNK cells complexed with 1000 trastuzumab molecule basal well ) \div ( luminescence of target control well ) ] \times 100 \%$$

$$Percentage of lysed target cell in “ oNK cells complexed with 3000 trastuzumab molecule and target experimental well ” ( \% ) = 1 - [ ( luminescence of oNK cells complexed with 3000 trastuzumab molecule and target experimental well - luminescence of oNK cells complexed with 3000 trastuzumab molecule basal well ) \div ( luminescence of target control well ) ] \times 100 \%$$

$$Percentage of lysed target cell in “ oNK cells complexed with 6000 trastuzumab molecule and target experimental well ” ( \% ) = 1 - [ ( luminescence of oNK cells complexed with 6000 trastuzumab molecule and target experimental well - luminescence of oNK cells complexed with 6000 trastuzumab molecule basal well ) \div ( luminescence of target control well ) ] \times 100 \%$$

$$Percentage of lysed target cell in “ oNK cells complexed with 12000 trastuzumab molecule and target experimental well ” ( \% ) = 1 - [ ( luminescence of oNK cells complexed with 12000 trastuzumab molecule and target experimental well - luminescence of oNK cells complexed with 12000 trastuzumab molecule basal well ) \div ( luminescence of target control well ) ] \times 100 \%$$

The results of the present invention suggest that:
compare to Ctrl-oNK cells, oNK cells complexed with at least 1000 trastuzumab molecules per cell (for example, complexed with 1000, 3000, 3500, 6000, or 12000 trastuzumab molecules per cell) can kill more BT474 cells (p<0.05);
in contrast, compare to Ctrl-oNK cells, oNK cells complexed with 1000˜3000 trastuzumab molecules per cell can not kill more trastuzumab-resistant BT474 Clone 5 cells; and oNK cells complexed with more than 3000 trastuzumab molecules per cell (for example, complexed with 3500, 6000, or 12000 trastuzumab molecules per cell) can kill more trastuzumab-resistant BT474 Clone 5 cells compare to Ctrl-oNK cells (p<0.05).

Moreover, the results of the present invention suggest that if the forementioned trastuzumab-complexed human CD16⁺ natural killer cells are replaced with cetuximab-complexed human CD16⁺ natural killer cells, and BT474 and BT474 Clone 5 cells are replaced with target cells HCC827 and HT-29 (or HSC-4 and SAS), it would have results similar to the forementioned experiments. That is, the results of the present invention suggest that:
compare to Ctrl-oNK cells, oNK cells complexed with at least 1000 cetuximab molecules per cell (for example, complexed with 1000, 3000, 3500, 6000, or 12000 cetuximab molecules per cell) can kill more HCC827 cells (or HSC-4 cells) (p<0.05);
in contrast, compare to Ctrl-oNK cells, oNK cells complexed with 1000˜3000 cetuximab molecules per cell can not kill more cetuximab-resistant HT-29 cells (or SAS cells); and oNK cells complexed with more than 3000 cetuximab molecules per cell (for example, complexed with 3500, 6000, or 12000 cetuximab molecules per cell) can kill more cetuximab-resistant HT-29 cells (or SAS cells) compare to Ctrl-oNK cells (p<0.05).

Furthermore, the results of the present invention suggest that if the forementioned trastuzumab-complexed human CD16⁺ natural killer cells are replaced with rituximab-complexed human CD16⁺ natural killer cells, and BT474 and BT474 Clone 5 cells are replaced with target cells Raji and Raji-2R80 (or Raji-2RH), it would have results similar to the forementioned experiments. That is, the results of the present invention suggest that:
compare to Ctrl-oNK cells, oNK cells complexed with at least 1000 rituximab molecules per cell (for example, complexed with 1000, 3000, 3500, 6000, or 12000 rituximab molecules per cell) can kill more Raji cells (p<0.05);
in contrast, compare to Ctrl-oNK cells, oNK cells complexed with 1000˜3000 rituximab molecules per cell can not kill more rituximab-resistant Raji-2R80 (or Raji-2RH); and oNK cells complexed with more than 3000 rituximab molecules per cell (for example, complexed with 3500, 6000, or 12000 rituximab molecules per cell) can kill more rituximab-resistant Raji-2R80 (or Raji-2RH) in comparison to Ctrl-oNK cells (p<0.05).

In addition, the results of the present invention suggest that if the forementioned trastuzumab-complexed human CD16⁺ natural killer cells are replaced with avelumab-complexed human CD16⁺ natural killer cells, and BT474 and BT474 Clone 5 cells are replaced with target cells MDA-MB-231 and MDA-MB-468 (or H1650 and H2087), it would have results similar to the forementioned experiments. That is, the results of the present invention suggest that:
compare to Ctrl-oNK cells, oNK cells complexed with at least 1000 avelumab molecules per cell (for example, complexed with 1000, 3000, 3500, 6000, or 12000 avelumab molecules per cell) can kill more MDA-MB-231 cells (or H1650) (p<0.05);
in contrast, compare to Ctrl-oNK cells, oNK cells complexed with 1000˜3000 avelumab molecules per cell can not kill more avelumab-resistant MDA-MB-468 (or H2087); and oNK cells complexed with more than 3000 avelumab molecules per cell (for example, complexed with 3500, 6000, or 12000 avelumab molecules per cell) can kill more avelumab-resistant MDA-MB-468 (or H2087) in comparison to Ctrl-oNK cells (p<0.05).

### Embodiment 7-1-3 Effect of the Number of Complexed Ingredients on the Expression of Activation Marker and Cytotoxic Molecule in the Ingredient-Complexed Human CD16⁺ Natural Killer Cells after Co-Culture with Ingredient-Responsive or Ingredient-Resistant Target Cells

This embodiment comprised a 96-well cell culture plate to carry out cytotoxicity test, and the wells in 96-well cell culture plate were divided into:
oNK cells complexed with 0 trastuzumab molecule experimental wells.
oNK cells complexed with 1000 trastuzumab molecules experimental wells.
oNK cells complexed with 3000 trastuzumab molecules experimental wells.
oNK cells complexed with 6000 trastuzumab molecules experimental wells.
oNK cells complexed with 12000 trastuzumab molecules experimental wells.
target control wells, and
medium background control wells.

The target cells used in this embodiment were sensitive BT-474 (HTB-20, purchased from ATCC) or resistant BT-474 clone 5 cell lines (CRL-3247, purchased from ATCC), which are adherent human breast cancer cell lines.

BT-474 or BT-474 clone 5 target cells were seeded in the following wells:
oNK cells complexed with 0 trastuzumab molecule experimental wells.
oNK cells complexed with 1000 trastuzumab molecules experimental wells.
oNK cells complexed with 3000 trastuzumab molecules experimental wells.
oNK cells complexed with 6000 trastuzumab molecules experimental wells.
oNK cells complexed with 12000 trastuzumab molecules experimental wells, and
target control wells. Hence, each well-contained 10000 target cells, and was allowed to sit 30 minutes, then the cell culture plate was incubated under the condition of 37° C. and 5% carbon dioxide for two hours.

A sample of the 23-day cultured oNK cell suspension was added to the oNK cells complexed with 0 trastuzumab molecule experimental wells.

A sample of the human CD16⁺ natural killer cells that complexed with 500 to 1500 trastuzumab molecules per cell was added to the oNK cells complexed with 1000 trastuzumab molecule experimental wells.

A sample of the human CD16⁺ natural killer cells that complexed with 2500 to 3500 trastuzumab molecules per cell was added to the oNK cells complexed with 3000 trastuzumab molecule experimental wells.

A sample of the human CD16⁺ natural killer cells that complexed with 5500 to 6500 trastuzumab molecules per cell was added to the oNK cells complexed with 6000 trastuzumab molecule experimental wells.

A sample of the human CD16⁺ natural killer cells that complexed with 9000 to 15000 trastuzumab molecules per cell was added to the oNK cells complexed with 12000 trastuzumab molecule experimental wells.

The ratio of the number of effector cell to the number of target cell (BT474 or BT474 Clone 5 cells) in these wells was 1:1. The cell culture plate was placed in the incubator under the condition of 37° C. and 5% carbon dioxide for 5 hours.

The 96-well cell culture plate was centrifuged at 400×g for 5 minutes. The supernatants were removed and the cell pellets were washed with 0.2 mL of DPBS. Washed cell pellets were then stained with 100 uL of DPBS containing FITC-anti-human TNF alpha antibody (BioLegend. Cat.502906), anti-PE-anti-human CD56 antibody (BioLegend. Cat.). PE/Cy7-anti-human IFN gamma antibody (BioLegend, Cat.502528). Alexa Fluor 647-anti-human granzyme B antibody (BioLegend, Cat.), and APC-Cy7-anti-human CD107a antibody (BioLegend. Cat.328630) at 1:50 dilution for 10 minutes. The stained cells were centrifuged and washed with 0.2 mL of DPBS. The washed cells were resuspended with 0.5 mL of DPBS, and the CD56-positive gated populations were further analyzed for the percentage of TNF-α⁺, IFN-γ⁺, granzyme B⁺ and CD107a⁺. The mean fluorescence intensity of CD56-positive gated populations was also analyzed.

inventors of the present invention expect that:
oNK cells complexed with at least 1000 trastuzumab molecules per cell (for example, complexed with 1000, 3000, 3500, 6000, or 12000 trastuzumab molecules per cell) can express more TNF-α. IFN-γ, granzyme B and CD107a after co-culture with BT474 cells compare to that expressed by Ctrl-oNK cells after co-culture with BT474 cells (p<0.05);
in contrast, oNK cells complexed with 1000˜3000 trastuzumab molecules per cell can not express more TNF-α, IFN-γ, granzyme B and CD107a after co-culture with trastuzumab-resistant BT474 Clone 5 cells compare to that expressed by Ctrl-oNK cells after co-culture with BT474 Clone 5 cells; and oNK cells complexed with more than 3000 trastuzumab molecules per cell (for example, complexed with 3500, 6000, or 12000 trastuzumab molecules per cell) can express more TNF-α. IFN-γ, granzyme B and CD107a after co-culture with trastuzumab-resistant BT474 Clone 5 cells compare to that expressed by Ctrl-oNK cells after co-culture with BT474 Clone 5 cells

Inventors of the present invention expect that if the forementioned trastuzumab-complexed human CD16⁺ natural killer cells are replaced with cetuximab-complexed human CD16⁺ natural killer cells, and BT474 and BT474 Clone 5 cells are replaced with target cells HCC827 and HT-29 (or HSC-4 and SAS), it would have results similar to the forementioned experiments. That is, inventors of the present invention expect that:
oNK cells complexed with 1000 cetuximab molecules per cell (for example, complexed with 1000, 3000, 3500, 6000, or 12000 cetuximab molecules per cell) can express more TNF-α, IFN-γ, granzyme B and CD107a after co-culture with HCC827 cells (or HSC-4 cells) compare to that expressed by Ctrl-oNK cells after co-culture with HCC827 cells (or HSC-4 cells) (p<0.05);
in contrast, oNK cells complexed with 1000˜3000 cetuximab molecules per cell cannot express more TNF-α, IFN-γ, granzyme B and CD107a after co-culture with cetuximab-resistant HT-29 cells (or SAS cells) compare to that expressed by Ctrl-oNK cells after co-culture with cetuximab-resistant HT-29 cells (or SAS cells); and oNK cells complexed with more than 3000 cetuximab molecules per cell (for example, complexed with 3500, 6000, or 12000 cetuximab molecules per cell) can express more TNF-α, IFN-γ, granzyme B and CD107a after co-culture with cetuximab-resistant HT-29 cells (or SAS cells) (p<0.05).

Inventors of the present invention expect that if the forementioned trastuzumab-complexed human CD16⁺ natural killer cells are replaced with rituximab-complexed human CD16⁺ natural killer cells, and BT474 and BT474 Clone 5 cells are replaced with target cells Raji and Raji-2R80 (or Raji-2RH), it would have results similar to the forementioned experiments. That is, inventors of the present invention expect that:
oNK cells complexed with 1000 rituximab molecules per cell (for example, complexed with 1000, 3000, 3500, 6000, or 12000 rituximab molecules per cell) can express more TNF-α, IFN-γ, granzyme B and CD107a after co-culture with Raji cells compare to that expressed by Ctrl-oNK cells after co-culture with Raji cells (p<0.05);
in contrast, oNK cells complexed with 1000˜3000 rituximab molecules per cell cannot express more TNF-α, IFN-γ, granzyme B and CD107a after co-culture with rituximab-resistant Raji-2R80 (or Raji-2RH) compare to that expressed by Ctrl-oNK cells after co-culture with rituximab-resistant Raji-2R80 (or Raji-2RH); and oNK cells complexed with more than 3000 rituximab molecules per cell (for example, complexed with 3500, 6000, or 12000 rituximab molecules per cell) can express more TNF-α, IFN-γ, granzyme B and CD107a after co-culture with rituximab-resistant Raji-2R80 (or Raji-2RH) in comparison to that expressed by Ctrl-oNK cells after co-cultured with Raji-2R80 (p<0.05).

Inventors of the present invention expect that if the forementioned trastuzumab-complexed human CD16⁺ natural killer cells are replaced with avelumab-complexed human CD16⁺ natural killer cells, and BT474 and BT474 Clone 5 cells are replaced with target cells MDA-MB-231 and MDA-MB-468 (or H1650 and H2087), it would have results similar to the forementioned experiments. That is, inventors of the present invention expect that:
oNK cells complexed with at least 1000 avelumab molecules per cell (for example, complexed with 1000, 3000, 3500, 6000, or 12000 avelumab molecules per cell) can express more TNF alpha, IFN gamma, granzyme B and CD107a after co-culture with MDA-MB-231 cells (or H1650) compare to that expressed by Ctrl-oNK cells after co-culture with MDA-MB-231 cells (or H1650) (p<0.05);
in contrast, oNK cells complexed with 1000˜3000 avelumab molecules per cell cannot express more TNF-α, IFN-γ, granzyme B and CD107a after co-culture with avelumab-resistant MDA-MB-468 (or H2087) compare to that expressed by Ctrl-oNK cells after co-culture with avelumab-resistant MDA-MB-468 (or H2087); and oNK cells complexed with more than 3000 avelumab molecules per cell (for example, complexed with 3500, 6000, or 12000 avelumab molecules per cell) can express more TNF-α, IFN-γ, granzyme B and CD107a after co-culture with avelumab-resistant MDA-MB-468 (or H2087) in comparison to that expressed by Ctrl-oNK cells after co-cultured with MDA-MB-468 (or H2087) (p<0.05).

### Embodiment 7-1-4 Effect of the Number of Complexed Ingredients on the Migration of the Ingredient-Complexed Human CD16⁺ Natural Killer Cells

The outer chamber used in this embodiment was a 24-well plate (ThermoScientific, Cat. 142475). The inner chambers used in this embodiment were polycarbonate cell culture inserts (Millipore. Cat. PITP01250). There were six groups in this embodiment, which were:
(1) oNK cells complexed with 0 trastuzumab molecule groups: 2×10⁵ target cells (BT474 or BT474 Clone 5 cells) were seeded in the outer chamber. After 2 hours of attachment, polycarbonate cell culture inserts with pore size of 5 μm (Millicell cell culture insert) were inserted into the outer chamber, and 1×10⁶ cells in the 36-day cultured oNK cell suspension were seeded into each inner chamber for 19 hours of incubation.
(2) oNK cells complexed with 1000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human CD16⁺ natural killer cells that complexed with 500 to 1500 trastuzumab molecules per cell were seeded into each inner chamber for 19 hours of incubation.
(3) oNK cells complexed with 3000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human CD16⁺ natural killer cells that complexed with 2500 to 3500 trastuzumab molecules per cell were seeded into each inner chamber for 19 hours of incubation.
(4) oNK cells complexed with 6000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human CD16⁺ natural killer cells that complexed with 5500 to 6500 trastuzumab molecules per cell were seeded into each inner chamber for 19 hours of incubation.
(5) oNK cells complexed with 12000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human CD16⁺ natural killer cells that complexed with 9000 to 15000 trastuzumab molecules per cell were seeded into each inner chamber for 19 hours of incubation.
(6) oNK cells complexed with 30000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human CD16⁺ natural killer cells that complexed with 25000 to 35000 trastuzumab molecules per cell were seeded into each inner chamber for 19 hours of incubation.

Cells in the outer chamber were harvested and stained with PE-conjugated mouse anti-human CD56 (BioLegend, #304606) and PE-Cy5-conjugated CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended CD56⁺/CD3 gated cells was analyzed by flow cytometry.

The results of the present invention suggest that
compare to the amount of Ctrl-oNK cells moving to the outer chamber containing the BT474 cells, more amount of oNK cells complexed with at least 1000 trastuzumab molecules per cell (for example, complexed with 1000, 3000, 6000, 12000, or 30000 trastuzumab molecules per cell) in the inner chamber can pass through the membrane at the bottom of the polycarbonate cell culture inserts and move to the outer chamber containing the BT474 cells (p<0.05);
in contrast, compare to the amount of Ctrl-oNK cells moving to the outer chamber containing the trastuzumab-resistant BT474 Clone 5 cells, similar amount of oNK cells complexed with 1000˜3000 trastuzumab molecules per cell pass through the membrane at the bottom of the polycarbonate cell culture inserts to move to the outer chamber containing the trastuzumab-resistant BT474 Clone 5 cells, and more amount of oNK cells complexed with more than 3000 trastuzumab molecules per cell (for example, complexed with 6000, 12000, or 30000 trastuzumab molecules per cell) in the inner chamber can pass through the membrane at the bottom of the polycarbonate cell culture inserts and move to the outer chamber containing the trastuzumab-resistant BT474 Clone 5 cells (p<0.05).

Moreover, the results of the present invention suggest that if the forementioned trastuzumab-complexed human CD16⁺ natural killer cells are replaced with cetuximab-complexed human CD16⁺ natural killer cells, and BT474 and BT474 Clone 5 cells are replaced with target cells HCC827-luc and HT-29 (or HSC-4 and SAS), it would have results similar to the forementioned experiments. That is, the results of the present invention suggest that:
compare to the amount of Ctrl-oNK cells moving to the outer chamber containing the HCC827-luc cells (or HSC-4 cells), more amount of oNK cells complexed with at least 1000 cetuximab molecules per cell (for example, complexed with 1000, 3000, 6000, 12000, or 30000 cetuximab molecules per cell) can pass through the membrane at the bottom of the polycarbonate cell culture inserts and move to the outer chamber containing the HCC827-luc cells (or HSC-4 cells) (p<0.05);
in contrast, compare to the amount of Ctrl-oNK cells moving to the outer chamber containing the cetuximab-resistant HT-29 cells (or SAS cells), a similar amount of oNK cells complexed with 1000˜3000 cetuximab molecules per cell pass through the membrane at the bottom of the polycarbonate cell culture insert plates to move to the outer chamber containing cetuximab-resistant HT-29 cells (or SAS cells), and more amount of oNK cells complexed with more than 3000 cetuximab molecules per cell (for example, complexed with 6000, 12000, or 30000 cetuximab molecules per cell) in the inner chamber can pass through the membrane at the bottom of the polycarbonate cell culture inserts and move to the outer chamber containing the cetuximab-resistant HT-29 cells (or SAS cells) (p<0.05).

Furthermore, the results of the present invention suggest that if the forementioned trastuzumab-complexed human CD16⁺ natural killer cells are replaced with rituximab-complexed human CD16⁺ natural killer cells, and BT474 and BT474 Clone 5 cells are replaced with target cells Raji and Raji-2R80 (or Raji-2RH), it would have results similar to the forementioned experiments. That is, the results of the present invention suggest that:
compare to the amount of Ctrl-oNK cells moving to the outer chamber containing Raji cells, more amount of oNK cells complexed with at least 1000 rituximab molecules per cell (for example, complexed with 1000, 3000, 6000, 12000, or 30000 rituximab molecules per cell) can pass through the membrane at the bottom of the polycarbonate cell culture insert plates and move to the outer chamber containing Raji cells (p<0.05);
in contrast, compare to the amount of Ctrl-oNK cells moving to the outer chamber containing the rituximab-resistant Raji-2R80 (or Raji-2RH), a similar amount of oNK cells complexed with 1000˜3000 rituximab molecules per cell pass through the membrane at the bottom of the polycarbonate cell culture inserts to move to the outer chamber containing rituximab-resistant Raji-2R80 (or Raji-2RH), and more amount of oNK cells complexed with more than 3000 rituximab molecules per cell (for example, complexed with 6000, 12000, or 30000 rituximab molecules per cell) in the inner chamber can pass through the membrane at the bottom of the polycarbonate cell culture inserts and move to the outer chamber containing the rituximab-resistant Raji-2R80 (or Raji-2RH) (p<0.05).

In addition, the results of the present invention suggest that if the forementioned trastuzumab-complexed human CD16⁺ natural killer cells are replaced with avelumab-complexed human CD16⁺ natural killer cells, and BT474 and BT474 Clone 5 cells are replaced with target cells MDA-MB-231 and MDA-MB-468 (or H1650 and H2087), it would have results similar to the forementioned experiments. That is, the results of the present invention suggest that:
compare to the amount of Ctrl-oNK cells moving to the outer chamber containing MDA-MB-231 cells (or H1650), more amount of oNK cells complexed with at least 1000 avelumab molecules per cell (for example, complexed with 1000, 3000, 6000, 12000, or 30000 avelumab molecules per cell) can pass through the membrane at the bottom of the polycarbonate cell culture inserts and move to the outer chamber containing MDA-MB-231 cells (or H1650) (p<0.05).
in contrast, compare to the amount of Ctrl-oNK cells moving to the outer chamber containing the avelumab-resistant MDA-MB-468 (or H2087), a similar amount of oNK cells complexed with 1000˜3000 avelumab molecules per cell pass through the membrane at the bottom of the polycarbonate cell culture inserts to move to the outer chamber containing avelumab-resistant MDA-MB-468 (or H2087), and more amount of oNK cells complexed with more than 3000 avelumab molecules per cell (for example, complexed with 6000, 12000, or 30000 avelumab molecules per cell) in the inner chamber can pass through the membrane at the bottom of the polycarbonate cell culture inserts and move to the outer chamber containing the avelumab-resistant MDA-MB-468 (or H2087) (p<0.05).

### Embodiment 7-1-5 Effect of the Human CD16⁺ Natural Killer Cells Complexed with Different Number of Ingredients on the Migratory Capacity of the CD3⁺ T Cells

The outer chamber used in this embodiment was a 24-well plate (ThermoScientific. Cat. 142475). The inner chambers used in this embodiment were polycarbonate cell culture inserts with pore size of 3 μm (Millicell cell culture insert; Millipore, Cat. PITP01250).

There were six groups in this embodiment, which were:
(1) oNK cells complexed with 0 trastuzumab molecule groups: 4×10⁵ target cells (BT474 or BT474 Clone 5 cells) and 1×10⁶ cells in the 33-day cultured oNK cell suspension were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(2) oNK cells complexed with 1000 trastuzumab molecules groups: 4×10⁵ target cells (BT474 or BT474 Clone 5 cells) and 1×10⁶ human CD16⁺ natural killer cells that complexed with 500 to 1500 trastuzumab molecules per cell were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(3) oNK cells complexed with 3000 trastuzumab molecules groups: 4×10⁵ target cells (BT474 or BT474 Clone 5 cells) and 1×10⁶ human CD16⁺ natural killer cells that complexed with 2500 to 3500 trastuzumab molecules per cell were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(4) oNK cells complexed with 6000 trastuzumab molecules groups: 4×10⁵ target cells (BT474 or BT474 Clone 5 cells) and 1×10⁶ human CD16⁺ natural killer cells that complexed with 5500 to 6500 trastuzumab molecules per cell were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(5) oNK cells complexed with 12000 trastuzumab molecules groups: 4×10⁵ target cells (BT474 or BT474 Clone 5 cells) and 1×10⁶ human CD16⁺ natural killer cells that complexed with 9000 to 15000 trastuzumab molecules per cell were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(6) oNK cells complexed with 30000 trastuzumab molecules groups: 4×10⁵ target cells (BT474 or BT474 Clone 5 cells) and 1×10⁶ human CD16⁺ natural killer cells that complexed with 25000 to 35000 trastuzumab molecules per cell were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.

Cells in the outer chamber were harvested and stained with CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended CD3⁺ gated cells was analyzed by flow cytometry.

Inventors of the present invention expect that:

A large amount of CD3⁺ T cells in the inner chamber can pass through the membrane at the bottom of the polycarbonate cell culture inserts and move to the outer chamber containing the BT474 cells and oNK cells complexed with at least 1000 trastuzumab molecules per cell, but CD3⁺ T cells can not pass through the membrane at the bottom of the polycarbonate cell culture inserts to move to the outer chamber containing the trastuzumab-resistant BT474 Clone 5 cells and oNK cells complexed with small numbers of trastuzumab molecules per cell until the number of complexed trastuzumab reaches more than 3000 trastuzumab molecules per cell.

Moreover, inventors of the present invention expect that if the forementioned trastuzumab-complexed human CD16⁺ natural killer cells are replaced with cetuximab-complexed human CD16⁺ natural killer cells, and BT474 and BT474 Clone 5 cells are replaced with target cells HCC827 and HT-29 (or HSC-4 and SAS), it would have results similar to the forementioned experiments. That is, inventors of the present invention expect that a large amount of CD3⁺ T cells can pass through the membrane at the bottom of the polycarbonate cell culture inserts and move to the outer chamber containing the HCC827 cells (or HSC-4 cells) and oNK cells complexed with at least 1000 cetuximab molecules per cell, but CD3⁺ T cells can not pass through the membrane at the bottom of the polycarbonate cell culture inserts to move to the outer chamber containing cetuximab-resistant HT-29 cells (or SAS cells) and oNK cells complexed with small numbers of cetuximab molecules per cell until the number of complexed cetuximab reaches more than 3000 cetuximab molecules per cell.

Furthermore, inventors of the present invention expect that if the forementioned trastuzumab-complexed human CD16⁺ natural killer cells are replaced with rituximab-complexed human CD16⁺ natural killer cells, and BT474 and BT474 Clone 5 cells are replaced with target cells Raji and Raji-2R80 (or Raji-2RH), it would have results similar to the forementioned experiments. That is, inventors of the present invention expect that a large amount of CD3⁺ T cells can pass through the membrane at the bottom of the polycarbonate cell culture inserts and move to the outer chamber containing Raji cells and oNK cells complexed with at least 1000 rituximab molecules per cell, but CD3⁺ T cells can not pass through the membrane at the bottom of the polycarbonate cell culture inserts to move to the outer chamber containing rituximab-resistant Raji-2R80 (or Raji-2RH) and oNK cells complexed with small numbers of rituximab molecules per cell until the number of complexed rituximab reaches more than 3000 rituximab molecules per cell.

Inventors of the present invention expect that if the forementioned trastuzumab-complexed human CD16⁺ natural killer cells are replaced with avelumab-complexed human CD16⁺ natural killer cells, and BT474 and BT474 Clone 5 cells are replaced with target cells MDA-MB-231 and MDA-MB-468 (or H1650 and H2087), it would have results similar to the forementioned experiments. That is, inventors of the present invention expect that a large amount of CD3⁺ T cells can pass through the membrane at the bottom of the polycarbonate cell culture inserts and move to the outer chamber containing MDA-MB-231 cells (or H1650) and oNK cells complexed with at least 1000 avelumab molecules per cell, but CD3⁺ T cells can not pass through the membrane at the bottom of the polycarbonate cell culture inserts to move to the outer chamber containing avelumab-resistant MDA-MB-468 (or H2087) and oNK cells complexed with small numbers of avelumab molecules per cell until the number of complexed avelumab reaches more than 3000 avelumab molecules per cell.

### Embodiment 7-2: Effect of the Number of Complexed Ingredients on the Function of the Ingredient-Complexed Human Gamma Delta T Cells

### Embodiment 7-2-1 Preparation of Human Gamma Delta T Cells Complexed with Different Number of Ingredient

In this experiment, the cultured gamma delta T cell suspensions that was obtained by culturing for 16 days with the culture method disclosed in the Experiment 1-2-1 (16-day gamma delta T cell suspension of the present invention, refer to as 16-day gamma delta T cell suspension) were used to prepare human gamma delta T cells complexed with different number of ingredient (such as trastuzumab, cetuximab, rituximab, or avelumab).

After preparing cell-ssDNA conjugate by binding different amount of cell linkers to the cells in the 16-day gamma delta T cell suspension and then mixing the cell-ssDNA conjugate and the ingredient-ssDNA according to the chemical conjugation technology described in Embodiment 1-1-4, gamma delta T cells complexed with different number of ingredients (such as trastuzumab, cetuximab, rituximab, or avelumab) were obtained.

The ingredient-complexed human gamma delta T cell suspension was mixed with phycoerythrin-conjugated goat-anti-human Fab antibody (e.g., purchased form Jackson ImmunoResearch Laboratories, Inc.), so that the complexed ingredient (such as trastuzumab, cetuximab, rituximab, or avelumab) was interacted specifically with the phycoerythrin-conjugated goat-anti-human Fab antibody.

To convert mean fluorescence intensity of phycoerythrin-conjugated goat-anti-human Fab antibody into number of the ingredient (such as trastuzumab, cetuximab, rituximab, or avelumab) complexed on each of the human gamma delta T cell s, standard curves derived from Quantum™ Simply Cellular® kit (Bangs Laboratories, Inc, #815) were developed. There were 5 bottles of microspheres (+ populations “#1, #2, #3 and #4” coated with increasing amounts of anti-human IgG antibody, 1 uncoated blank) in the Quantum™ Simply Cellular® kit. Ten microliter of anti-human IgG antibody-bound microspheres, including #1, #2, #3 and #4 microsphere were individually incubated with 1 μg/mL of the ingredient (such as trastuzumab, cetuximab, rituximab, or avelumab) in total 0.1 mL reaction volume at room temperature for 30 minutes. For the blank microsphere, the similar procedure but without the addition of the ingredient (such as trastuzumab, cetuximab, rituximab, or avelumab) was performed. The trastuzumab-bound #1 to #1 microspheres and the blank microspheres were then detected by the above mentioned phycoerythrin-conjugated goat-anti-human Fab antibody. Microspheres were washed with 0.5 mL of DPBS and the suspension was centrifuged at 400×g at room temperature for 5 minutes. The supernatant was removed and the suspended QSC microspheres were analyzed by flow cytometry. Acquired mean fluorescence intensity value of each microsphere was inserted into respective columns of manufacturer-provided calculation sheet (QuickCal V2.3) to generate the corresponding standard curve following manufacturer's instruction. After individually developing the standard curve of the absolute number of each ingredient (such as trastuzumab, cetuximab, rituximab, or avelumab), 0, 1000±500, 3000±500, 6000±500, 12000±3000, 20000±5000, 30000±5000, 50000±5000, or 130000±5000 ingredient molecules (such as trastuzumab molecules, cetuximab molecules, rituximab molecules, or avelumab molecules) was next inserted to the QuickCal sheet to convert into corresponding fluorescence intensity value of the phycoerythrin-conjugated goat-anti-human Fab antibody-stained human gamma delta T cells that complexed with 0, 1000±500, 3000±500, 6000±500, 12000±3000, 20000±5000, 30000±5000, 50000±5000, or 130000±5000 ingredient molecules per cell.

The cell sorter (BD FACSMelody, BD FACSAria III. SONY SH800S, etc.) was used to isolate human gamma delta T cells that complexed with 0, 1000±500, 3000±500, 6000±500, 12000±3000, 20000±5000, 30000±5000, 50000±5000, or 130000±5000 ingredient molecules (such as trastuzumab molecules, cetuximab molecules, rituximab molecules, or avelumab molecules) per cell.

There were nine kinds of effector cells used in each of the Embodiments 7-2-2 to 7-2-5:
(1) gdT cells complexed with 0 trastuzumab molecule: cells in the 16-day gamma delta T cell suspension were used in this group.
(2) gdT cells complexed with 1000 trastuzumab molecules: human gamma delta T cells that complexed with 500 to 1500 trastuzumab molecules per cell were used in this group.
(3) gdT cells complexed with 3000 trastuzumab molecules: human gamma delta T cells that complexed with 2500 to 3500 trastuzumab molecules per cell were used in this group.
(4) gdT cells complexed with 6000 trastuzumab molecules: human gamma delta T cells that complexed with 5500 to 6500 trastuzumab molecules per cell were used in this group.
(5) gdT cells complexed with 12000 trastuzumab molecules: human gamma delta T cells that complexed with 9000 to 15000 trastuzumab molecules per cell were used in this group.
(6) gdT cells complexed with 20000 trastuzumab molecules: human gamma delta T cells that complexed with 15000 to 25000 trastuzumab molecules per cell were used in this group.
(7) gdT cells complexed with 30000 trastuzumab molecules: human gamma delta T cells that complexed with 25000 to 35000 trastuzumab molecules per cell were used in this group.
(8) gdT cells complexed with 50000 trastuzumab molecules: human gamma delta T cells that complexed with 45000 to 55000 trastuzumab molecules per cell were used in this group.
(9) gdT cells complexed with 130000 trastuzumab molecules: human gamma delta T cells that complexed with 125000 to 135000 trastuzumab molecules per cell were used in this group.

### Embodiment 7-2-2 Effect of the Number of Complexed Ingredients on the Cytotoxic Function of the Ingredient-Complexed Human Gamma Delta T Cells

CellTiter-Glo® Luminescent Cell Viability Assay (Promega. USA) was used in this embodiment to detect the cytotoxic ability of the effector cells toward target cells. First, wells in a CELLSTAR® 96 well plates (Cat. Number 655083, purchased from Greiner) were divided into:
(1) target control well.
(2) gdT cells complexed with 0 trastuzumab molecule basal well.
(3) gdT cells complexed with 0 trastuzumab molecule and target experimental well.
(4) gdT cells complexed with 1000 trastuzumab molecule basal well.
(5) gdT cells complexed with 1000 trastuzumab molecule and target experimental well.
(6) gdT cells complexed with 3000 trastuzumab molecule basal well.
(7) gdT cells complexed with 3000 trastuzumab molecule and target cell experimental well.
(8) gdT cells complexed with 6000 trastuzumab molecule basal well, and
(9) gdT cells complexed with 6000 trastuzumab molecule and target cell experimental well.
(10) gdT cells complexed with 12000 trastuzumab molecule basal well, and
(11) gdT cells complexed with 12000 trastuzumab molecule and target cell experimental well.

The target cells used in this Experiment were \textcircled{1} human breast cancer cell line BT474 (HTB-20, purchased from ATCC), or \textcircled{2} BT474's derived trastuzumab-resistant clone BT474 Clone 5 (CRL-3247, purchased from ATCC).

Target cells (BT474 or BT474 Clone 5 cells) were seeded in:
the target control wells.
the gdT cells complexed with 0 trastuzumab molecule and target experimental wells.
the gdT cells complexed with 1000 trastuzumab molecule and target experimental wells.
the gdT cells complexed with 3000 trastuzumab molecule and target experimental wells.
the gdT cells complexed with 6000 trastuzumab molecule and target experimental wells, and
the gdT cells complexed with 12000 trastuzumab molecule and target experimental wells.
so that each well-contained 5000 target cells.

A sample of the 16-day gamma delta T cell suspension (Ctrl-gdT cells) was added to:
the gdT cells complexed with 0 trastuzumab molecule basal wells, and
the gdT cells complexed with 0 trastuzumab molecule and target experimental wells.

A sample of the human gamma delta T cells that complexed with 500 to 1500 trastuzumab molecules per cell was added to:
the gdT cells complexed with 1000 trastuzumab molecule basal wells, and
the gdT cells complexed with 1000 trastuzumab molecule and target experimental wells.

A sample of the human gamma delta T cells that complexed with 2500 to 3500 trastuzumab molecules per cell was added to:
the gdT cells complexed with 3000 trastuzumab molecule basal wells, and
the gdT cells complexed with 3000 trastuzumab molecule and target experimental wells.

A sample of the human gamma delta T cells that complexed with 5500 to 6500 trastuzumab molecules per cell was added to:
the gdT cells complexed with 6000 trastuzumab molecule basal wells, and
the gdT cells complexed with 6000 trastuzumab molecule and target experimental wells.

A sample of the human gamma delta T cells that complexed with 9000 to 15000 trastuzumab molecules per cell was added to:
the gdT cells complexed with 12000 trastuzumab molecule basal wells, and
the gdT cells complexed with 12000 trastuzumab molecule and target experimental wells.

The ratio of the number of effector cell to the number of target cell (BT474 or BT474 Clone 5 cells) in these wells was the same in each group.

The CELLSTAR® 96 well plates were incubated at 37° C. in 5% CO₂ for 4 hours. After 4 hours of incubation, the culture were mixed with 50 μL of CellTiter® Glo substrate (provided in the CellTiter-Glo® luminescent cell viability assay kit. Promega. Cat. G7570) and incubated at room temperature without light for 12 minutes. The luminescence of each well was measured and recorded by luminescence plate reader (Synergy H1. BioTek Instruments, USA).

Wherein, the greater the number of live cells remained in the well, the higher the luminescence detected by the Synergy H1 system. Therefore, the luminescence can be used to convert the percentage of target cells that are lysed in the experimental well. The formula used to convert the luminescence to the percentage of target cells that are lysed in the experimental wells are as follows:

$$Percentage of lysed target cell in “ gdT cells complexed with 0 trastuzumab molecule and target experimental well ” ( \% ) = 1 - [ ( luminescence of gdT cells complexed with 0 trastuzumab molecule and target experimental well - luminescence of gdT cells complexed with 0 trastuzumab molecule basal well ) \div ( luminescence of target control well ) ] \times 100 \%$$

$$Percentage of lysed target cell in “ gdT cells complexed with 1000 trastuzumab molecule and target experimental well ” ( \% ) = 1 - [ ( luminescence of gdT cells complexed with 1000 trastuzumab molecule and target experimental well - luminescence of gdT cells complexed with 1000 trastuzumab molecule basal well ) \div ( luminescence of target control well ) ] \times 100 \%$$

$$Percentage of lysed target cell in “ gdT cells complexed with 3000 trastuzumab molecule and target experimental well ” ( \% ) = 1 - [ ( luminescence of gdT cells complexed with 3000 trastuzumab molecule and target experimental well - luminescence of gdT cells complexed with 3000 trastuzumab molecule basal well ) \div ( luminescence of target control well ) ] \times 100 \%$$

$$Percentage of lysed target cell in “ gdT cells complexed with 6000 trastuzumab molecule and target experimental well ” ( \% ) = 1 - [ ( luminescence of gdT cells complexed with 6000 trastuzumab molecule and target experimental well - luminescence of gdT cells complexed with 6000 trastuzumab molecule basal well ) \div ( luminescence of target control well ) ] \times 100 \%$$

$$Percentage of lysed target cell in “ gdT cells complexed with 12000 trastuzumab molecule and target experimental well ” ( \% ) = 1 - [ ( luminescence of gdT cells complexed with 12000 trastuzumab molecule and target experimental well - luminescence of gdT cells complexed with 12000 trastuzumab molecule basal well ) \div ( luminescence of target control well ) ] \times 100 \%$$

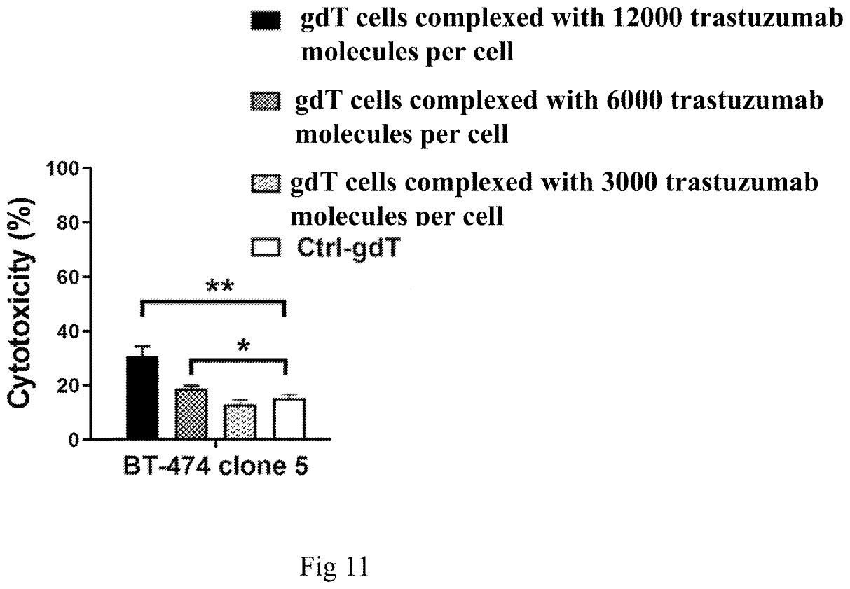

The results show that:
compare to Ctrl-gdT cells, gdT cells complexed with at least 1000 trastuzumab molecules per cell (for example, complexed with 1000, 3000, 3500, 6000, or 12000 trastuzumab molecules per cell) can kill more BT474 cells (p<0.05);
   in contrast, please refer to FIG. 11, compare to Ctrl-gdT cells, gdT cells complexed with 1000˜3000 trastuzumab molecules per cell can not kill more trastuzumab-resistant BT474 Clone 5 cells; and gdT cells complexed with more than 3000 trastuzumab molecules per cell (for example, complexed with 3500, 6000, or 12000 trastuzumab molecules per cell) can kill more trastuzumab-resistant BT474 Clone 5 cells compare to Ctrl-gdT cells (p<0.05).

Moreover, the results of the present invention suggest that if the forementioned trastuzumab-complexed human gamma delta T cells are replaced with cetuximab-complexed human gamma delta T cells, and BT474 and BT474 Clone 5 cells are replaced with target cells HCC827 and HT-29 (or HSC-4 and SAS), it would have results similar to the forementioned experiments. That is, the results of the present invention suggest that:
compare to Ctrl-oNK cells, gdT cells complexed with at least 1000 cetuximab molecules per cell (for example, complexed with 1000, 3000, 3500, 6000, or 12000 cetuximab molecules per cell) can kill more HCC827 cells (or HSC-4 cells) (p<0.05);
in contrast, compare to Ctrl-gdT cells, gdT cells complexed with 1000˜3000 cetuximab molecules per cell can not kill more cetuximab-resistant HT-29 cells (or SAS cells); and gdT cells complexed with more than 3000 cetuximab molecules per cell (for example, complexed with 3500, 6000, or 12000 cetuximab molecules per cell) can kill more cetuximab-resistant HT-29 cells (or SAS cells) compare to Ctrl-gdT cells (p<0.05).

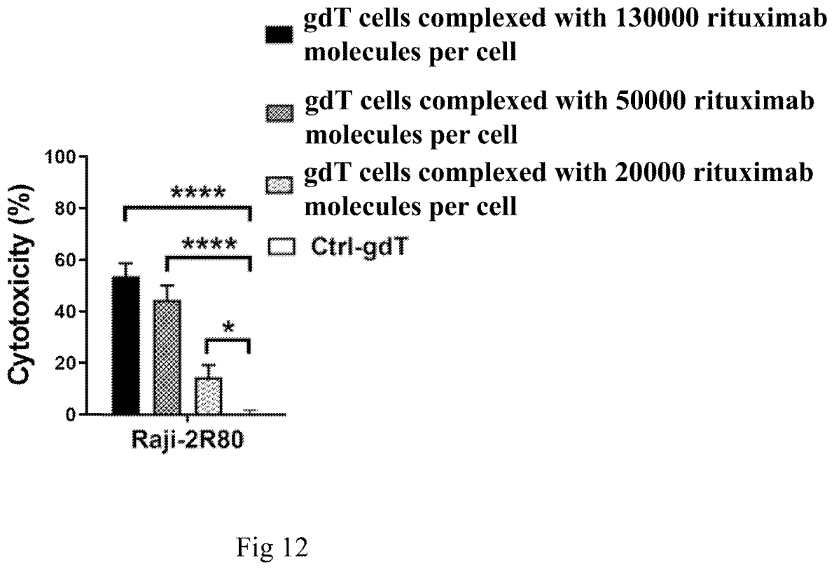

Furthermore, the results show that if the forementioned trastuzumab-complexed human gamma delta T cells are replaced with rituximab-complexed human gamma delta T cells (anti-CD 20 antibody-complexed human gamma delta T cells), and BT474 and BT474 Clone 5 cells are replaced with target cells Raji and Raji-2R80 (or Raji-2RH), it would have results similar to the forementioned experiments. That is, the results show that:
compare to Ctrl-gdT cells, gdT cells complexed with at least 1000 rituximab molecules per cell (for example, complexed with 1000, 3000, 3500, 6000, 12000, 20000, 50000, or 130000 rituximab molecules per cell) can kill more Raji cells (p<0.05);
in contrast, please refer to FIG. 12, compare to Ctrl-gdT cells, gdT cells complexed with 1000˜3000 rituximab molecules per cell can not kill more rituximab-resistant Raji-2R80 (or Raji-2RH); and gdT cells complexed with more than 3000 rituximab molecules per cell (for example, complexed with 3500, 6000, 12000, 20000, 50000, or 130000 rituximab molecules per cell) can kill more rituximab-resistant Raji-2R80 (or Raji-2RH) (p<0.05).

In addition, the results of the present invention suggest that if the forementioned trastuzumab-complexed human gamma delta T cells are replaced with avelumab-complexed human gamma delta T cells, and BT474 and BT474 Clone 5 cells are replaced with target cells MDA-MB-231 and MDA-MB-468 (or H1650 and H2087), it would have results similar to the forementioned experiments. That is, the results of the present invention suggest that:
compare to Ctrl-gdT cells, gdT cells complexed with at least 1000 avelumab molecules per cell (for example, complexed with 1000, 3000, 3500, 6000, or 12000 avelumab molecules per cell) can kill more MDA-MB-231 cells (or H1650) (p<0.05);
in contrast, compare to Ctrl-gdT cells, gdT cells complexed with 1000˜3000 avelumab molecules per cell can not kill more avelumab-resistant MDA-MB-468 (or H2087); and gdT cells complexed with more than 3000 avelumab molecules per cell (for example, complexed with 3500, 6000, or 12000 avelumab molecules per cell) can kill more avelumab-resistant MDA-MB-468 (or H2087).

### Embodiment 7-2-3 Effect of the Number of Complexed Ingredients on the Expression of Activation Marker and Cytotoxic Molecule in the Ingredient-Complexed Human Gamma Delta T Cells after Co-Culture with Ingredient-Responsive or Ingredient-Resistant Target Cells

This embodiment comprised a 96-well cell culture plate to carry out cytotoxicity test, and the wells in 96-well cell culture plate were divided into:
gdT cells complexed with 0 trastuzumab molecule experimental wells.
gdT cells complexed with 1000 trastuzumab molecules experimental wells.
gdT cells complexed with 3000 trastuzumab molecules experimental wells.
gdT cells complexed with 6000 trastuzumab molecules experimental wells.
gdT cells complexed with 12000 trastuzumab molecules experimental wells, and
medium background control wells.

The target cells used in this embodiment were sensitive BT-474 (HTB-20, purchased from ATCC) or resistant BT-474 clone 5 cell lines (CRL-3247, purchased from ATCC), which are adherent human breast cancer cell lines.

BT-474 or BT-474 clone 5 target cells were seeded in the following wells:
gdT cells complexed with 0 trastuzumab molecule experimental wells.
gdT cells complexed with 1000 trastuzumab molecules experimental wells.
gdT cells complexed with 3000 trastuzumab molecules experimental wells.
gdT cells complexed with 6000 trastuzumab molecules experimental wells.
gdT cells complexed with 12000 trastuzumab molecules experimental wells, and
target control wells. Hence, each well-contained 5000 target cells, and was allowed to sit 30 minutes, then the cell culture plate was incubated under the condition of 37° C. and 5% carbon dioxide for two hours.

A sample of the 16-day gamma delta T cell suspension was added to the gdT cells complexed with 0 trastuzumab molecule experimental wells.

A sample of the human gamma delta T cells that complexed with 500 to 1500 trastuzumab molecules per cell was added to the gdT cells complexed with 1000 trastuzumab molecule experimental wells.

A sample of the human gamma delta T cells that complexed with 2500 to 3500 trastuzumab molecules per cell was added to the gdT cells complexed with 3000 trastuzumab molecule experimental wells.

A sample of the human gamma delta T cells that complexed with 5500 to 6500 trastuzumab molecules per cell was added to the gdT cells complexed with 6000 trastuzumab molecule experimental wells.

A sample of the human gamma delta T cells that complexed with 9000 to 15000 trastuzumab molecules per cell was added to the gdT cells complexed with 12000 trastuzumab molecule experimental wells.

The ratio of the number of effector cell to the number of target cell (BT474 or BT474 Clone 5 cells) in these wells was the same in each group. The cell culture plate was placed in the incubator under the condition of 37° C. and 5% carbon dioxide for 5 hours.

The 96-well cell culture plate was centrifuged at 400×g for 5 minutes. The supernatants were removed and the cell pellets were washed with 0.2 mL of DPBS. Washed cell pellets were then stained with 100 μL of DPBS containing FITC-anti-human TNF alpha antibody (BioLegend. Cat.502906), anti-PE-anti-human CD56 antibody (BioLegend, Cat.). PE/Cy7-anti-human IFN gamma antibody (BioLegend. Cat.502528). Alexa Fluor 647-anti-human granzyme B antibody (BioLegend. Cat.), and APC-Cy7-anti-human CD107a antibody (BioLegend, Cat.328630) at 1:50 dilution for 10 minutes. The stained cells were centrifuged and washed with 0.2 mL of DPBS. The washed cells were resuspended with 0.5 mL of DPBS, and the CD56-positive gated populations were further analyzed for the percentage of TNF alpha⁺, IFN gamma⁺, granzyme B⁺ and CD107a⁺. The mean fluorescence intensity of CD56-positive gated populations was also analyzed.

inventors of the present invention expect that:
gdT cells complexed with at least 1000 trastuzumab molecules per cell (for example, complexed with 1000, 3000, 3500, 6000, or 12000 trastuzumab molecules per cell) can express more TNF alpha, IFN gamma, granzyme B and CD107a after co-culture with BT474 cells compare to that expressed by Ctrl-gdT cells after co-culture with BT474 cells (p<0.05);
in contrast, gdT cells complexed with 1000˜3000 trastuzumab molecules per cell can not express more TNF alpha, IFN gamma, granzyme B and CD107a after co-culture with trastuzumab-resistant BT474 Clone 5 cells compare to that expressed by Ctrl-gdT cells after co-culture with BT474 Clone 5 cells; and gdT cells complexed with more than 3000 trastuzumab molecules per cell (for example, complexed with 3500, 6000, or 12000 trastuzumab molecules per cell) can express more TNF-α. IFN-γ, granzyme B and CD107a after co-culture with trastuzumab-resistant BT474 Clone 5 cells compare to that expressed by Ctrl-gdT cells after co-culture with BT474 Clone 5 cells.

Moreover, inventors of the present invention expect that if the forementioned trastuzumab-complexed human gamma delta T cells are replaced with cetuximab-complexed human gamma delta T cells, and BT474 and BT474 Clone 5 cells are replaced with target cells HCC827 and HT-29 (or HSC-4 and SAS), it would have results similar to the forementioned experiments. That is, inventors of the present invention expect that:
gdT cells complexed with at least 1000 cetuximab molecules per cell (for example, complexed with 1000, 3000, 3500, 6000, or 12000 cetuximab molecules per cell) can express more TNF alpha, IFN gamma, granzyme B and CD107a after co-culture with HCC827 cells (or HSC-4 cells) compare to that expressed by Ctrl-gdT cells after co-culture with HCC827 cells (or HSC-4 cells) (p<0.05);
in contrast, gdT cells complexed with 1000˜3000 cetuximab molecules per cell can not express more TNF alpha, IFN gamma, granzyme B and CD107a after co-culture with cetuximab-resistant HT-29 cells (or SAS cells) compare to that expressed by Ctrl-gdT cells after co-culture with cetuximab-resistant HT-29 cells (or SAS cells); and gdT cells complexed with more than 3000 cetuximab molecules per cell (for example, complexed with 3500, 6000, or 12000 cetuximab molecules per cell) can express more TNF-α, IFN-γ, granzyme B and CD107a after co-culture with cetuximab-resistant HT-29 cells (or SAS cells) (p<0.05).

Furthermore, inventors of the present invention expect that if the forementioned trastuzumab-complexed human gamma delta T cells are replaced with rituximab-complexed human gamma delta T cells, and BT474 and BT474 Clone 5 cells are replaced with target cells Raji and Raji-2R80 (or Raji-2RH), it would have results similar to the forementioned experiments. That is, inventors of the present invention expect that:
gdT cells complexed with at least 1000 rituximab molecules per cell (for example, complexed with 1000, 3000, 3500, 6000, or 12000 rituximab molecules per cell) can express more TNF alpha, IFN gamma, granzyme B and CD107a after co-culture with Raji cells compare to that expressed by Ctrl-gdT cells after co-culture with Raji cells;
in contrast, gdT cells complexed with 1000˜3000 rituximab molecules per cell can not express more TNF alpha, IFN gamma, granzyme B and CD107a after co-culture with rituximab-resistant Raji-2R80 (or Raji-2RH) compare to that expressed by Ctrl-gdT cells after co-culture with rituximab-resistant Raji-2R80 (or Raji-2RH); and gdT cells complexed with more than 3000 rituximab molecules per cell (for example, complexed with 3500, 6000, or 12000 rituximab molecules per cell) can express more TNF-α, IFN-γ, granzyme B and CD107a after co-culture with rituximab-resistant Raji-2R80 (or Raji-2RH) (p<0.05).

In addition, inventors of the present invention expect that if the forementioned trastuzumab-complexed human gamma delta T cells are replaced with avelumab-complexed human gamma delta T cells, and BT474 and BT474 Clone 5 cells are replaced with target cells MDA-MB-231 and MDA-MB-468 (or H1650 and H2087), it would have results similar to the forementioned experiments. That is, inventors of the present invention expect that:
gdT cells complexed with at least 1000 avelumab molecules per cell (for example, complexed with 1000, 3000, 3500, 6000, or 12000 avelumab molecules per cell) can express more TNF alpha, IFN gamma, granzyme B and CD107a after co-culture with MDA-MB-231 cells (or H1650) compare to that expressed by Ctrl-gdT cells after co-culture with MDA-MB-231 cells (or H1650) (p<0.05);
in contrast, gdT cells complexed with 1000˜3000 avelumab molecules per cell can not express more TNF alpha, IFN gamma, granzyme B and CD107a after co-culture with avelumab-resistant MDA-MB-468 (or H2087) compare to that expressed by Ctrl-gdT cells after co-culture with avelumab-resistant MDA-MB-468 (or H2087); and gdT cells complexed with more than 3000 avelumab molecules per cell (for example, complexed with 3500, 6000, or 12000 avelumab molecules per cell) can express more TNF-α, IFN-γ, granzyme B and CD107a after co-culture with avelumab-resistant MDA-MB-468 (or H2087) (p<0.05).

### Embodiment 7-2-4 Effect of the Number of Complexed Ingredients on the Migration of the Ingredient-Complexed Human Gamma Delta T Cells

The outer chamber used in this embodiment was a 24-well plate (ThermoScientific, Cat. 142475). The inner chambers used in this embodiment were polycarbonate cell culture inserts (Millipore. Cat. PITP01250). There were six groups in this embodiment, which were:
(1) gdT cells complexed with 0 trastuzumab molecule groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ cells in the 16-day gamma delta T cell suspension were seeded into each inner chamber for 19 hours of incubation.
(2) gdT cells complexed with 1000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human gamma delta T cells that complexed with 500 to 1500 trastuzumab molecules per cell were seeded into each inner chamber for 19 hours of incubation.
(3) gdT cells complexed with 3000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human gamma delta T cells that complexed with 2500 to 3500 trastuzumab molecules per cell were seeded into each inner chamber for 19 hours of incubation.
(4) gdT cells complexed with 6000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human gamma delta T cells that complexed with 5500 to 6500 trastuzumab molecules per cell were seeded into each inner chamber for 19 hours of incubation.
(5) gdT cells complexed with 12000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human gamma delta T cells that complexed with 9000 to 15000 trastuzumab molecules per cell were seeded into each inner chamber for 19 hours of incubation.
(6) gdT cells complexed with 30000 trastuzumab molecules groups: 2×10⁵ target cells (BT474 or BT474 Clone 5 cells) were seeded in the outer chamber. After 2 hours of attachment, polycarbonate cell culture inserts with pore size of 3 μm (Millicell cell culture insert) were inserted into the outer chamber, and 1×10⁶ human gamma delta T cells that complexed with 25000 to 35000 trastuzumab molecules per cell were seeded into each inner chamber for 19 hours of incubation.

Cells in the outer chamber were harvested and stained with FITC-conjugated mouse anti-human TCRVd2 (BioLegend, #331406) and PE-Cy5-conjugated CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended TCRVd2⁺/CD3⁺ gated cells was analyzed by flow cytometry.

The results of the present invention suggest that:

Compare to the amount of Ctrl-gdT cells moving to the outer chamber containing the BT474 cells, more amount of gdT cells complexed with at least 1000 trastuzumab molecules per cell (for example, complexed with 1000, 3000, 6000, 12000, or 30000 trastuzumab molecules per cell) in the inner chamber can pass through the membrane at the bottom of the polycarbonate cell culture inserts and move to the outer chamber containing the BT474 cells (p<0.05);
in contrast, compare to the amount of Ctrl-gdT cells moving to the outer chamber containing the trastuzumab-resistant BT474 Clone 5 cells, similar amount of gdT cells complexed with 1000˜3000 trastuzumab molecules per cell pass through the membrane at the bottom of the polycarbonate cell culture inserts to move to the outer chamber containing the trastuzumab-resistant BT474 Clone 5 cells, and more amount of gdT cells complexed with more than 3000 trastuzumab molecules per cell (for example, complexed with 6000, 12000, or 30000 trastuzumab molecules per cell) in the inner chamber can pass through the membrane at the bottom of the polycarbonate cell culture inserts and move to the outer chamber containing the trastuzumab-resistant BT474 Clone 5 cells (p<0.05).

Moreover, the results of the present invention suggest that that if the forementioned trastuzumab-complexed human gamma delta T cells are replaced with cetuximab-complexed human gamma delta T cells, and BT474 and BT474 Clone 5 cells are replaced with target cells NCI-H508 and HT-29 (or HSC-4 and SAS), it would have results similar to the forementioned experiments. That is, the results of the present invention suggest that:
compare to the amount of Ctrl-gdT cells moving to the outer chamber containing the HCC827 cells (or HSC-4 cells), more amount of gdT cells complexed with at least 1000 cetuximab molecules per cell (for example, complexed with 1000, 3000, 6000, 12000, or 30000 cetuximab molecules per cell) can pass through the membrane at the bottom of the polycarbonate cell culture inserts and move to the outer chamber containing the HCC827 cells (or HSC-4 cells) (p<0.05);
in contrast, compare to the amount of Ctrl-gdT cells moving to the outer chamber containing the cetuximab-resistant HT-29 cells (or SAS cells), a similar amount of gdT cells complexed with 1000˜3000 cetuximab molecules per cell pass through the membrane at the bottom of the polycarbonate cell culture inserts to move to the outer chamber containing cetuximab-resistant HT-29 cells (or SAS cells), and more amount of gdT cells complexed with more than 3000 cetuximab molecules per cell (for example, complexed with 6000, 12000, or 30000 cetuximab molecules per cell) in the inner chamber can pass through the membrane at the bottom of the polycarbonate cell culture inserts and move to the outer chamber containing the cetuximab-resistant HT-29 cells (or SAS cells) (p<0.05).

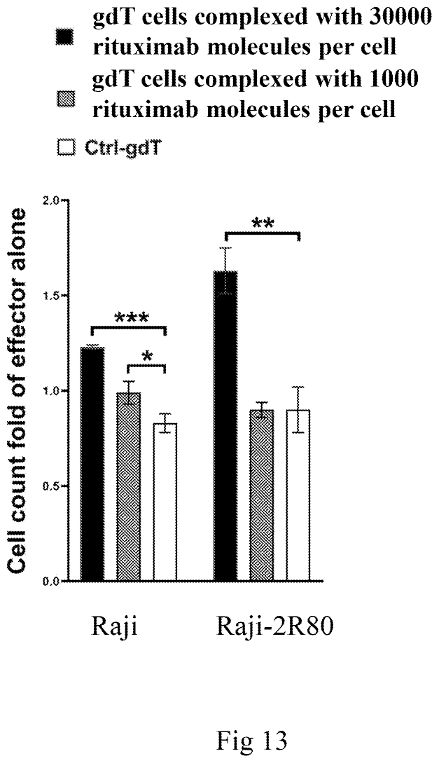

Moreover, please refer to FIG. 13. FIG. 13 shows that if the forementioned trastuzumab-complexed human gamma delta T cells are replaced with rituximab-complexed human gamma delta T cells, and BT474 and BT474 Clone 5 cells are replaced with target cells Raji and Raji-2R80 (or Raji-2RH), it would have results similar to the forementioned experiments. That is. FIG. 13 shows that:
compare to the amount of Ctrl-gdT cells moving to the outer chamber containing Raji cells, more amount of gdT cells complexed with at least 1000 rituximab molecules per cell (for example, complexed with 1000, 3000, 6000, 12000, or 30000 rituximab molecules per cell) can pass through the membrane at the bottom of the polycarbonate cell culture inserts and move to the outer chamber containing Raji cells (p<0.05);
in contrast, compare to the amount of Ctrl-gdT cells moving to the outer chamber containing the rituximab-resistant Raji-2R80 (or Raji-2RH), a similar amount of gdT cells complexed with 1000˜3000 rituximab molecules per cell pass through the membrane at the bottom of the polycarbonate cell culture inserts to move to the outer chamber containing rituximab-resistant Raji-2R80 (or Raji-2RH), and more amount of gdT cells complexed with more than 3000 rituximab molecules per cell (for example, complexed with 6000, 12000, or 30000 rituximab molecules per cell) in the inner chamber can pass through the membrane at the bottom of the polycarbonate cell culture inserts and move to the outer chamber containing the rituximab-resistant Raji-2R80 (or Raji-2RH) (p<0.05).

Furthermore, the results of the present invention suggest that if the forementioned trastuzumab-complexed human gamma delta T cells are replaced with avelumab-complexed human gamma delta T cells, and BT474 and BT474 Clone 5 cells are replaced with target cells MDA-MB-231 and MDA-MB-468 (or H1650 and H2087), it would have results similar to the forementioned experiments. That is, the results of the present invention suggest that:
compare to the amount of Ctrl-gdT cells moving to the outer chamber containing MDA-MB-231 cells (or H1650), more amount of gdT cells complexed with at least 1000 avelumab molecules per cell (for example, complexed with 1000, 3000, 6000, 12000, or 30000 avelumab molecules per cell) can pass through the membrane at the bottom of the polycarbonate cell culture inserts and move to the outer chamber containing MDA-MB-231 cells (or H1650) (p<0.05).
in contrast, compare to the amount of Ctrl-gdT cells moving to the outer chamber containing the avelumab-resistant MDA-MB-468 (or H2087), a similar amount of gdT cells complexed with 1000˜3000 avelumab molecules per cell pass through the membrane at the bottom of the polycarbonate cell culture inserts to move to the outer chamber containing avelumab-resistant MDA-MB-468 (or H2087), and more amount of gdT cells complexed with more than 3000 avelumab molecules per cell (for example, complexed with 6000, 12000, or 30000 avelumab molecules per cell) in the inner chamber can pass through the membrane at the bottom of the polycarbonate cell culture inserts and move to the outer chamber containing the avelumab-resistant MDA-MB-468 (or H2087) (p<0.05).

### Embodiment 7-2-5 Effect of the Human Gamma Delta T Cells Complexed with Different Number of Ingredients on the Migratory Capacity of the CD3⁺ T Cells and CD56⁺CD3-NK Cells

The outer chamber used in this embodiment was a 24-well plate (ThermoScientific, Cat. 142475). The inner chambers used in this embodiment were polycarbonate cell culture inserts with pore size of 3 μm (Millicell cell culture insert; Millipore, Cat. PITP01250).

There were 14 groups comprising group (1), group (2), group (3), group (3)′, group (+), group (4)′, group (5), group (5)′, group (6), group (6)′, group (7), group (7)′, group (8), and group (8)′ in this embodiment, which were:
(1) Medium group (group (1)): basal medium of gdT cells (RPMI-1640, purchased from Sigma-Aldrich, ThermoFisher Scientific. ATCC, etc.) were placed in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells and CD56⁺CD3⁻ in PBMC cells.
(2) Target cell alone group (group (2)): 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells CD56⁺CD3⁻ in PBMC cells.
(3) gdT cells complexed with 0 trastuzumab molecule groups: 1×10⁶ cells in the 16-day gamma delta T cell suspension in the presence (group (3)) or absence (group (3)′) of 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T and CD56⁺CD3-cells in PBMC cells.
(4) gdT cells complexed with 1000 trastuzumab molecules groups: 1×10⁶ human gamma delta T cells that complexed with 500 to 1500 trastuzumab molecules per cell in the presence (group (4)) or absence (group (4)′) of 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T and CD56⁺CD3-cells in PBMC cells.
(5) gdT cells complexed with 3000 trastuzumab molecules groups: 1×10⁶ human gamma delta T cells that complexed with 2500 to 3500 trastuzumab molecules per cell in the presence (group (5)) or absence (group (5)′) of 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T and CD56⁺CD3-NK cells in PBMC cells.
(6) gdT cells complexed with 6000 trastuzumab molecules groups: 1×10⁶ human gamma delta T cells that complexed with 5500 to 6500 trastuzumab molecules per cell in the presence (group (6)) or absence (group (6)′) of 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T and CD56⁺CD3-NK cells in PBMC cells.
(7) gdT cells complexed with 12000 trastuzumab molecules groups: 1×10⁶ human gamma delta T cells that complexed with 9000 to 15000 trastuzumab molecules per cell in the presence (group (7)) or absence (group (7)′) of 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T and CD56⁺CD3-NK cells in PBMC cells.
(8) gdT cells complexed with 30000 trastuzumab molecules groups: 1×10⁶ human gamma delta T cells that complexed with 25000 to 35000 trastuzumab molecules per cell in the presence (group (8)) or absence (group (8)′) of 4×10⁵ target cells (BT474 or BT474 Clone 5 cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T and CD56⁺CD3-cells in PBMC cells.

Cells in the outer chamber were harvested and stained with CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended CD3⁺ and CD56⁺CD3-gated cells was analyzed by flow cytometry.

Inventors of the present invention expect that:

A large amount of CD3⁺ T cells in the inner chamber can pass through the membrane at the bottom of the polycarbonate cell culture inserts and move to the outer chamber containing the BT474 cells and gdT cells complexed with at least 1000 trastuzumab molecules per cell, but CD3⁺ T cells can not pass through the membrane at the bottom of the polycarbonate cell culture inserts to move to the outer chamber containing the trastuzumab-resistant BT474 Clone 5 cells and gdT cells complexed with small numbers of trastuzumab molecules per cell until the number of complexed trastuzumab reaches more than 3000 trastuzumab molecules per cell.

Moreover, inventors of the present invention expect that if the forementioned trastuzumab-complexed human gamma delta T cells are replaced with cetuximab-complexed human gamma delta T cells, and BT474 and BT474 Clone 5 cells are replaced with target cells NCI-H508 and HT-29 (or HSC-4 and SAS), it would have results similar to the forementioned experiments. That is, inventors of the present invention expect that a large amount of CD3⁺ T cells can pass through the membrane at the bottom of the polycarbonate cell culture inserts and move to the outer chamber containing the HCC827 cells (or HSC-4 cells) and gdT cells complexed with at least 1000 cetuximab molecules per cell, but CD3⁺ T cells can not pass through the membrane at the bottom of the polycarbonate cell culture inserts to move to the outer chamber containing cetuximab-resistant HT-29 cells (or SAS cells) and gdT cells complexed with small numbers of cetuximab molecules per cell until the number of complexed cetuximab reaches more than 3000 cetuximab molecules per cell.

Furthermore, inventors of the present invention expect that if the forementioned trastuzumab-complexed human gamma delta T cells are replaced with rituximab-complexed human gamma delta T cells, and BT474 and BT474 Clone 5 cells are replaced with target cells Raji and Raji-2R80 (or Raji-2RH), it would have results similar to the forementioned experiments. That is, inventors of the present invention expect that a large amount of CD3⁺ T cells can pass through the membrane at the bottom of the polycarbonate cell culture inserts and move to the outer chamber containing Raji cells and gdT cells complexed with at least 1000 rituximab molecules per cell, but CD3⁺ T cells can not pass through the membrane at the bottom of the polycarbonate cell culture inserts to move to the outer chamber containing rituximab-resistant Raji-2R80 (or Raji-2RH) and gdT cells complexed with small numbers of rituximab molecules per cell until the number of complexed rituximab reaches more than 3000 rituximab molecules per cell.

In addition, inventors of the present invention expect that if the forementioned trastuzumab-complexed human gamma delta T cells are replaced with avelumab-complexed human gamma delta T cells, and BT474 and BT474 Clone 5 cells are replaced with target cells MDA-MB-231 and MDA-MB-468 (or H1650 and H2087), it would have results similar to the forementioned experiments. That is, inventors of the present invention expect that a large amount of CD3⁺ T cells can pass through the membrane at the bottom of the polycarbonate cell culture inserts and move to the outer chamber containing MDA-MB-231 cells (or H1650) and gdT cells complexed with at least 1000 avelumab molecules per cell, but CD3⁺ T cells can not pass through the membrane at the bottom of the polycarbonate cell culture inserts to move to the outer chamber containing avelumab-resistant MDA-MB-468 (or H2087) and gdT cells complexed with small numbers of avelumab molecules per cell until the number of complexed avelumab reaches more than 3000 avelumab molecules per cell.

### Embodiment 8: Cytotoxic Cells Complexed with an Ingredient that was Concluded to be Ineffective or without Adequate Efficacy in Treating a Disease, and the Uses of the Ingredient-Complexed Cytotoxic Cells in Treating the Disease

Codrituzumab (GC33) is an antibody against glypican-3 (GPC3). Glypican-3 (GPC3) is overexpressed in some kinds of tumors such as hepatocellular carcinoma. Even though codrituzumab is successful in phase I clinical trial, it failed in Phase II clinical trial and thus not an FDA-approved ingredient for the treatment of tumors such as hepatocellular carcinoma (Takahiro Nishida and Hiroaki Kataoka, 2019).

The following describes a specific embodiment of preparing codrituzumab-complexed natural killer cells and codrituzumab-complexed gamma delta T cells, as well as the uses thereof, but the application of the invention is not limited thereto, which means any ingredient that was concluded to be ineffective or without adequate efficacy in treating a disease to be conjugated with any cytotoxic cells are intended to be included in the scope of the invention.

### Embodiment 8-1: Human CD16⁺ Natural Killer Cells Complexed with an Ingredient that was Concluded to be Ineffective or without Adequate Efficacy in Treating a Disease, and the Uses of the Ingredient-Complexed Human CD16⁺ Natural Killer Cells in Treating the Disease

### Embodiment 8-1-1: Cytotoxic Function of the Codrituzumab-Complexed Human CD16⁺ Natural Killer Cells to Kill Human Hepatocellular Carcinoma Cell Line

xCELLigence Real Time Cell Analysis System (xCELLigence RTCA system, ACEA Biosciences Inc., USA) was used in this embodiment to detect the cytotoxic ability of the effector cell toward target cells:
(1) The wells in xCELLigence E-Plate were divided into control wells, codrituzumab C2 experimental well, codrituzumab C5 experimental well, codrituzumab C10 experimental well, Ctrl-oNK ET2 experimental well. Ctrl-oNK ET5 experimental well, Ctrl-oNK ET10 experimental well, Ctrl-oNK and codrituzumab ET2 experimental well. Ctrl-oNK and codrituzumab ET5 experimental well. Ctrl-oNK and codrituzumab ET10 experimental well. ACE-GPC3-ONK ET2 experimental well. ACE-GPC3-ONK ET5 experimental well, ACE-GPC3-ONK ET10 experimental well, and target cell maximum lysis control well.
(2) The effector cell used in this Experiment were \textcircled{1} the cells in the 23-day cultured oNK cell suspension, or \textcircled{2} the cells in the codrituzumab-complexed human CD16⁺ natural killer cell suspension; and
(3) The target cells used in this Experiment were human hepatocellular carcinoma cell line HuH-7 (JCRB0403, purchased from JCRB).

HuH-7 target cells were seeded in the control wells, codrituzumab C2 experimental well, codrituzumab C5 experimental well, codrituzumab C10 experimental well. Ctrl-oNK ET2 experimental well. Ctrl-oNK ET5 experimental well. Ctrl-oNK ET10 experimental well, Ctrl-oNK and codrituzumab ET2 experimental well. Ctrl-oNK and codrituzumab ET5 experimental well. Ctrl-oNK and codrituzumab ET10 experimental well, ACE-GPC3-ONK ET2 experimental well, ACE-GPC3-ONK ET5 experimental well. ACE-GPC3-ONK ET10 experimental well, and target cell maximum lysis control well; hence, each well-contained 20000 target cells, and was allowed to sit 30 minutes.

40000, 100000, or 200000 cells in the codrituzumab-complexed human CD16⁺ natural killer cell suspension was added to the ACE-GPC3-ONK ET2 experimental well, ACE-GPC3-ONK ET5 experimental well, and ACE-GPC3-ONK ET10 experimental well respectively; hence, the ratio of the number of effector cell (the total cells in the codrituzumab-complexed human CD16⁺ natural killer cell suspension) to the number of HuH-7 cells (target cells) was 2, 5, and 10.

Both of 40000, 100000, or 200000 cells in the 23-day cultured oNK suspension and 1.10, 2.75, or 5.5 ng of codrituzumab (purchased from Creative Biolabs) were added to the “Ctrl-oNK and codrituzumab ET2 experimental well”. “Ctrl-oNK and codrituzumab ET5 experimental well”, or “Ctrl-oNK and codrituzumab ET10 experimental well” respectively. Therefore, the ratio of the number of effector cell (the total cells in the 23-day cultured oNK suspension) to the number of HuH-7 cells (target cells) was 2, 5, or 10; the amount of codrituzumab in the “Ctrl-oNK and codrituzumab ET2 experimental well”. “Ctrl-oNK and codrituzumab ET5 experimental well”, or “Ctrl-oNK and codrituzumab ET10 experimental well” was respectively same as the total amount of the codrituzumab linked to the cells in the “ACE-GPC3-ONK ET2 experimental well”. “ACE-GPC3-ONK ET5 experimental well”, or “ACE-GPC3-ONK ET10 experimental well”.

1.10, 2.75, or 5.5 ng of codrituzumab (purchased from Creative Biolabs) was respectively added to the “codrituzumab C2 experimental well”. “codrituzumab C5 experimental well”, or “codrituzumab C10 experimental well”. Therefore, the amount of codrituzumab in the “codrituzumab C2 experimental well”. “codrituzumab C5 experimental well”, or “codrituzumab C10 experimental well” was respectively same as the total amount of the codrituzumab linked to the cells in the “ACE-GPC3-ONK ET2 experimental well”. “ACE-GPC3-ONK ET5 experimental well”, or “ACE-GPC3-ONK ET10 experimental well”.

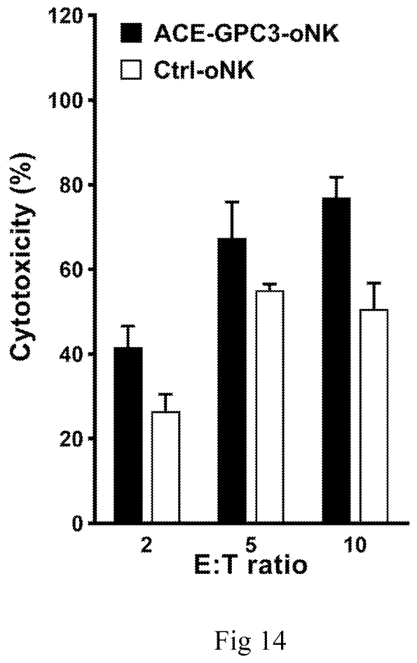

Added one tenth equal volume of lysis buffer to the sample into target cell maximum lysis control well; no sample or lysis buffer was added to control well. The xCELLigence E-Plate was placed in the xCELLigence Real Time Cell Analysis System to detect real time change in the cell index (CI) under the condition of 37° C. and 5% carbon dioxide. Please refer to FIG. 14. FIG. 14 shows that the killing effect of human CD16⁺ natural killer cells complexed with codrituzumab (ACE-GPC3-ONK) is significantly higher than the killing effect of human CD16⁺ natural killer cells (Ctrl-oNK).

Inventors of the present invention expect: By comparing among the cytotoxic efficacy of the codrituzumab C2 experimental well (or codrituzumab C5 experimental well or codrituzumab C10 experimental) and Ctrl-oNK ET2 experimental well (or Ctrl-oNK ET5 experimental well or Ctrl-oNK ET10 experimental well), it is expected that the codrituzumab and Ctrl-oNK in the “ACE-GPC3-ONK ET2 experimental well” (or “ACE-GPC3-ONK ET5 experimental well” or “ACE-GPC3-ONK ET10 experimental well”) surprisingly manifest synergy in the cytotoxic efficacy, and the killing capacity of the cells in the “ACE-GPC3-ONK ET2 experimental well” (or “ACE-GPC3-ONK ET5 experimental well” or “ACE-GPC3-oNK ET10 experimental well”) was significantly higher than that of the cells in the “Ctrl-oNK and Codrituzumab ET2 experimental well” (or “Ctrl-oNK and Codrituzumab ET5 experimental well” or “Ctrl-oNK and Codrituzumab ET10 experimental well”).

### Embodiment 8-1-2: Cytotoxicity of Codrituzumab-Complexed Human CD16⁺ Natural Killer Cells Against Hepatocellular Carcinoma (a Solid Tumor) in a Subject

Luciferase-expressing hepatocellular carcinoma cell line HuH-7 (JCRB1600, purchased from JCRB) was intraperitoneally injected into each of the 25 female NSG mice (Jackson Laboratory) on Day 0. The mice were divided into 5 groups randomly.
(1) Mice in the Ctrl-oNK group were treated with 5000000 cells in the 24-day cultured oNK cell suspension on Day 0, 3, 7, 10, 14, and 17.
(2) Mice in the ACE-GPC3-ONK group were treated with 5000000 cells in the codrituzumab-complexed human CD16⁺ natural killer cell suspension on Day 0, 3, 7, 10, 14 and 17.
(3) Mice in the Ctrl-oNK and codrituzumab group were treated with 5000000 cells in the 23-day cultured oNK cell suspension and 2.75 ng of codrituzumab (purchased from Creative BioLabs) on Day 0, 3, 7, 10, 14 and 17. Therefore, the amount of codrituzumab administered into the mice in the Ctrl-oNK and codrituzumab group (2.75 ng of codrituzumab) was same as the total amount of the codrituzumab linked to the cells that were administered into the mice in the ACE-GPC3-ONK group.
(4) Mice in the codrituzumab group were treated with 2.75 ng of codrituzumab on Day 0, 3, 7, 10, 14 and 17.
(6) Mice in the control group were treated with Vehicle (cell medium only, such as cell culture medium, DMEM culture medium or XVIVO 10 culture medium described in Experiment 1-1-1) on Day 0, 3, 7, 10, 14 and 17.

Luminescence was detected by AMI HTX (Spectral Imaging) on Day 0, 3, 7, 10, 14 and 17 and weekly after Day 17 until the end of the experiment.

Inventor of the present invention expect:
(1) The luminescence images of mice show that: bioluminescence image of mice treated with cells in the codrituzumab-complexed human CD16⁺ natural killer cell suspension demonstrates significant reduction. Therefore, the ingredient-complexed cytotoxic cells of the present invention could treat solid tumor in a subject as well as treat abnormal cells located in the immunosuppressive microenvironment such as solid tumor.
(2) Compare to the cytotoxic efficacy in the mice of “Ctrl-oNK group” and “codrituzumab group”, it is expected that the codrituzumab and Ctrl-oNK surprisingly manifest synergy in the cytotoxic efficacy in the mice of “ACE-GPC3-ONK group”, and the cytotoxic efficacy in the mice of “ACE-GPC3-oNK group” was significantly higher than that in the “Ctrl-oNK and codrituzumab group”.

### Embodiment 8-1-3: Effect of the Codrituzumab-Complexed Human CD16⁺ Natural Killer Cells on the Migratory Capacity of the CD3⁺ T Cells

There were four groups in this embodiment, which were (1) medium group, (2) HuH-7 group, (3) ACE-GPC3-oNK group, and (4) HuH-7 and ACE-GPC3-oNK group.
(1) Medium group: cell medium (such as cell culture medium, DMEM culture medium or XVIVO 10 culture medium described in Experiment 1-1-1) was added into the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(2) HuH-7 group: 4×10⁵ HuH-7 cells (Target cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cell.
(3) ACE-GPC3-ONK group: 1×10⁶ cells in the codrituzumab-complexed human CD16⁺ natural killer cell suspension cell medium (such as cell culture medium, DMEM culture medium or XVIVO 10 culture medium described in Experiment 1-1-1) was added into the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(4) HuH-7 and ACE-GPC3-oNK group: 4×10⁵ HuH-7 cells (Target cells) and 1×10⁶ cells in the codrituzumab-complexed human CD16⁺ natural killer cell suspension were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells

Cells in the outer chamber were harvested and stained with CD3 antibodies at room temperature for 10 minutes while avoiding the light. Cell mixtures were then centrifuged at room temperature at 400×g for 3 minutes. Supernatant was removed, and cell pellet was resuspended with 1 ml of DPBS. Centrifugation was repeated, and 0.5 mL of DPBS-resuspended CD3⁺ gated cells was analyzed by flow cytometry.

Inventors of the present invention expect that the ingredient-complexed cytotoxic cells in the outer chamber (lesion simulation) that comprises target cells such as HuH-7 cells would significantly increase the migratory capacity of CD3⁺ T cells into the lesion. By comparing among the CD3⁺ T cells migration efficacy of the HuH-7 group and the ACE-GPC3-oNK group, it is expected that the target cells (such as HuH-7) and ingredient-complexed cytotoxic cells in the outer chamber of the HuH-7 and ACE-GPC3-oNK group surprisingly manifest synergy in the CD3⁺ T cells migration efficacy.

### Embodiment 8-2: Human Gamma Delta T Cells Complexed with an Ingredient that was Concluded to be Ineffective or without Adequate Efficacy in Treating a Disease, and the Uses of the Ingredient-Complexed Human Gamma Delta T Cells in Treating the Disease

### Embodiment 8-2-1: Cytotoxic Function of the Codrituzumab-Complexed Human Gamma Delta T Cells to Kill Human Hepatocellular Carcinoma Cell Line

xCELLigence Real Time Cell Analysis System (xCELLigence RTCA system, ACEA Biosciences Inc., USA) was used in this embodiment to detect the cytotoxic ability of the effector cell toward target cells:
(1) The wells in xCELLigence E-Plate were divided into control wells, codrituzumab C2 experimental well, codrituzumab C5 experimental well, codrituzumab C10 experimental well, Ctrl-gdT ET2 experimental well. Ctrl-gdT ET5 experimental well. Ctrl-gdT ET10 experimental well. Ctrl-gdT and codrituzumab ET2 experimental well. Ctrl-gdT and codrituzumab ET5 experimental well. Ctrl-gdT and codrituzumab ET10 experimental well, ACE-gdT-GPC3 ET2 experimental well, ACE-gdT-GPC3 ET5 experimental well. ACE-gdT-GPC3 ET10 experimental well, and target cell maximum lysis control well.
(2) The effector cell used in this Experiment were \textcircled{1} the cells in the 16-day gamma delta T cell suspension, or \textcircled{2} the cells in the codrituzumab-complexed human gamma delta T cell suspension; and
(3) The target cells used in this Experiment were human hepatocellular carcinoma cell line HuH-7 (JCRB0403, purchased from JCRB).

HuH-7 target cells were seeded in the control wells, codrituzumab C2 experimental well, codrituzumab C5 experimental well, codrituzumab C10 experimental well. Ctrl-gdT ET2 experimental well, Ctrl-gdT ET5 experimental well. Ctrl-gdT ET10 experimental well. Ctrl-gdT and codrituzumab ET2 experimental well. Ctrl-gdT and codrituzumab ET5 experimental well. Ctrl-gdT and codrituzumab ET10 experimental well. ACE-gdT-GPC3 ET2 experimental well, ACE-gdT-GPC3 ET5 experimental well. ACE-gdT-GPC3 ET10 experimental well, and target cell maximum lysis control well; hence, each well-contained 20000 target cells, and was allowed to sit 30 minutes.

40000, 100000, or 200000 cells in the codrituzumab-complexed human gamma delta T cell suspension was added to the ACE-gdT-GPC3 ET2 experimental well. ACE-gdT-GPC3 ET5 experimental, and ACE-gdT-GPC3 ET10 experimental well respectively; hence, the ratio of the number of effector cell (the total cells in the codrituzumab-complexed human gamma delta T cell suspension) to the number of HuH-7 cells (target cells) was 2, 5, and 10.

All of 40000, 100000 or 200000 cells in the 16-day gamma delta T cell suspension and 1.10, 2.75, or 5.5 ng of codrituzumab (purchased from Creative BioLabs) were added to the “Ctrl-gdT and codrituzumab ET2 experimental well”. “Ctrl-gdT and codrituzumab ET5 experimental well”, or “Ctrl-gdT and codrituzumab ET10 experimental well” respectively. Therefore, the ratio of the number of effector cell (the total cells in the 16-day gamma delta T cell suspension) to the number of HuH-7 cells (target cells) was 2, 5 or 10; the amount of codrituzumab in the “Ctrl-gdT and codrituzumab ET2 experimental well”. “Ctrl-gdT and codrituzumab ET5 experimental well”, or “Ctrl-gdT and codrituzumab ET10 experimental well” was respectively same as the total amount of the codrituzumab linked to the cells in the “ACE-gdT-GPC3 ET2 experimental well”. “ACE-gdT-GPC3 ET5 experimental well”, or “ACE-gdT-GPC3 ET10 experimental well”.

1.10, 2.75, or 5.5 ng of codrituzumab (purchased from Creative BioLabs) was respectively added to the “codrituzumab C2 experimental well”. “codrituzumab C5 experimental well”, or “codrituzumab C10 experimental well”. Therefore, the amount of codrituzumab in the “codrituzumab C2 experimental well”. “codrituzumab C5 experimental well”, or “codrituzumab C10 experimental well” was respectively same as the total amount of the codrituzumab linked to the cells in the “ACE-gdT-GPC3 ET2 experimental well”. “ACE-gdT-GPC3 ET5 experimental well”, or “ACE-gdT-GPC3 ET10 experimental well”.

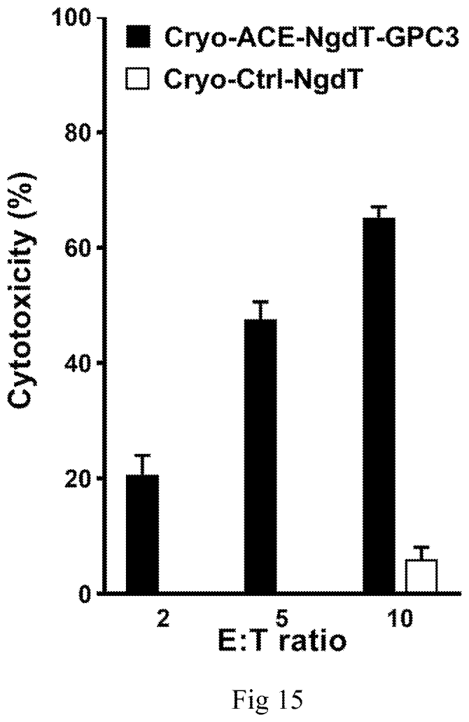

Added one tenth equal volume of lysis buffer to the sample into target cell maximum lysis control well; no sample or lysis buffer was added to control well. The xCELLigence E-Plate was placed in the xCELLigence Real Time Cell Analysis System to detect real time change in the cell index (CI) under the condition of 37° C. and 5% carbon dioxide. Please refer to FIG. 15. FIG. 15 shows that the killing effect of human gamma delta T cells complexed with codrituzumab (Cryo-ACE-NgdT-GPC3) is significantly higher than the killing effect of human gamma delta T cells (Cryo-Ctrl-NgdT).

Inventors of the present invention expect: By comparing among the cytotoxic efficacy of the codrituzumab C2 experimental well (or codrituzumab C5 experimental well or codrituzumab C10 experimental well) and Ctrl-gdT ET2 experimental well (or Ctrl-gdT ET5 experimental well or Ctrl-gdT ET10 experimental well), it is expected that the codrituzumab and Ctrl-gdT in the “ACE-gdT-GPC3 ET2 experimental well” (or “ACE-gdT-GPC3 ET5 experimental well” or “ACE-gdT-GPC3 ET10 experimental well”) unexpectedly manifest synergy in the cytotoxic efficacy, and the killing capacity of the cells in the “ACE-gdT-GPC3 ET2 experimental well” (or “ACE-gdT-GPC3 ET5 experimental well” or “ACE-gdT-GPC3 ET10 experimental well”) was significantly higher than that of the cells in the “Ctrl-gdT and codrituzumab ET2 experimental well” (or “Ctrl-gdT and codrituzumab ET5 experimental well” or “Ctrl-gdT and codrituzumab ET10 experimental well”).

### Embodiment 8-2-2: Cytotoxicity of Codrituzumab-Complexed Human Gamma Delta T Cells Against Hepatocellular Carcinoma (a Solid Tumor) in a Subject

Luciferase-expressing hepatocellular carcinoma cell line HuH-7 (JCRB1600, purchased from JCRB) was intraperitoneally injected into each of the 25 female NSG mice (Jackson Laboratory) on Day 0. The mice were divided into 5 groups.
(1) Mice in the Ctrl-gdT group were treated with 5000000 cells in the 16-day gamma delta T cell suspension on Day 0, 3, 7, 10, 14 and 17.
(2) Mice in the ACE-gdT-GPC3 group were treated with 5000000 cells in the codrituzumab-complexed human gamma delta T cell suspension on Day 0, 3, 7, 10, 14 and 17.
(3) Mice in the Ctrl-gdT and codrituzumab group were treated with 5000000 cells in the 16-day gamma delta T cell suspension and 2.75 ng of codrituzumab (purchased from Creative BioLabs) on Day 0, 3, 7, 10, 14, and 17. Therefore, the amount of codrituzumab to be administered into the mice in the Ctrl-gdT and codrituzumab group (2.75 ng of codrituzumab) was same as the total amount of the codrituzumab linked to the cells that were administered into the mice in the ACE-gdT-GPC3 group.
(4) Mice in the codrituzumab group were treated with 2.75 ng of codrituzumab on Day 0, 3, 7, 10 14, and 17.
(5) Mice in the control group were treated with Vehicle (cell medium only, such as complete growth medium described in Experiment 1-2-1) on Day 0, 3, 7, 10, 14, and 17.

Luminescence was detected by AMI HTX (Spectral Imaging) on Day 0, 3 and 7 and weekly after Day 7 until the end of the experiment.

Inventor of the present invention expect:
(1) The luminescence images of mice show that: bioluminescence image of mice treated with cells in the codrituzumab-complexed human gamma delta T cell suspension demonstrate significant reduction. Therefore, the ingredient-complexed cytotoxic cells of the present invention could treat solid tumor in a subject as well as treat abnormal cells located in the immunosuppressive microenvironment such as solid tumor.
(2) Compare to the cytotoxic efficacy in the mice of “Ctrl-gdT group” and “codrituzumab group”, it is expected that the codrituzumab and Ctrl-gdT unexpectedly manifest synergy in the cytotoxic efficacy in the mice of “ACE-gdT-GPC3 group”, and the cytotoxic efficacy in the mice of “ACE-gdT-GPC3 group” was significantly higher than that in the “Ctrl-gdT and codrituzumab group”.

### Embodiment 8-2-3: Effect of the Codrituzumab-Complexed Human Gamma Delta T Cells on the Migratory Capacity of the CD3⁺ T Cells

There were four groups in this embodiment, which were (1) medium group, (2) HuH-7 group, (3) ACE-gdT-GPC3 group, and (4) HuH-7 and ACE-gdT-GPC3 group.
(1) Medium group: cell medium (such as complete growth medium described in Experiment 1-2-1) was added into the outer chamber. After 2 hours, inner chamber was inserted into the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(2) HuH-7 group: 4×10⁵ HuH-7 cells (Target cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(3) ACE-gdT-GPC3 group: 1×10⁶ cells in the codrituzumab-complexed human gamma delta T cell suspension were seeded into the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.
(4) HuH-7 and ACE-gdT-GPC3 group: 1×10⁶ cells in the codrituzumab-complexed human gamma delta T cell suspension and 4×10⁵ HuH-7 cells (Target cells) were seeded in the outer chamber for 19 hours of incubation. The supernatant was transferred into a new outer chamber. Then 1×10⁶ donor-derived PBMC cells were seeded into the inner chamber placed in the new outer chamber for subsequent 3-hr of incubation to evaluate the migration of CD3⁺ T cells in PBMC cells.

Cells in the outer chamber were harvested and stained with CD3 antibodies at room temperature for 10 minutes while avoiding the light. Cell mixtures were then centrifuged at room temperature at 400×g for 3 minutes. Supernatant was removed, and cell pellet was resuspended with 1 mL of DPBS. Centrifugation was repeated, and 0.5 mL of DPBS-resuspended CD3⁺ gated cells was analyzed by flow cytometry.

Inventors of the present invention expect that the ingredient-complexed cytotoxic cells in the outer chamber (lesion simulation) that comprises target cells such as HuH-7 cells would significantly increase the migratory capacity of CD3⁺ T cells into the lesion. By comparing among the CD3⁺ T cells migration efficacy of the HuH-7 group and the ACE-gdT-GPC3 group, it is expected that the target cells (such as HuH-7) and ingredient-complexed cytotoxic cells in the outer chamber of the HuH-7 and ACE-gdT-GPC3 group surprisingly manifest synergy in the CD3⁺ T cells migration efficacy.

From the embodiments of the present invention, the immune system of subjects, and the pharmacological mechanism of ingredients, those skilled in the art would understand that all of the ingredients complexed with (or conjugated with, or linked to) all of the cytotoxic cells based on the disclosure, teaching, or suggestion of the present invention can be used for improving the effectiveness of said ingredients (such as FDA-approved drugs or ingredients that were successful in the Phase I trial but failed in Phase II or Phase III trials) in treating abnormal cells that are resistant, refractory, insensitive, non-responsive, or inadequately responsive to the ingredient. Therefore, ingredient-complexed cytotoxic cells of the present invention can be used in treating various kinds of abnormal cells associated with various kinds of diseases such as hyperproliferative diseases, advanced stage diseases, HIV or other viral infectious diseases, fungi infectious diseases, bacteria infectious diseases, protozoan infectious diseases, autoimmune diseases, neuronal diseases, hematopoietic cell-related diseases, metabolic syndromes, and pathogenic diseases.

The foregoing descriptions are merely the preferred embodiments of the present invention and are not intended to limit the scope of the patent application of the present invention. Therefore, any alteration or modification that does not depart from the spirits disclosed herein should be included within the scope of the patent application of the present invention.
Reference 1—J Rey 1. D Olive, G Sébahoun. T O'Callaghan. Régis T Costello, 2005. New insights into cytotoxic effector cells. Bull Cancer, 92 (11): 935-943.
Reference 2—I Lupu. N Voiculescu. N Bacalbasa. I Cojocaru. V Vrancian, and C Giurcaneanu, 2016. Cutaneous complications of molecular targeted therapy used in oncology. J Med Life. 9 (1): 19-25.
Reference 3—Lee M. Ellis and Daniel J. Hicklin, 2009. Resistance to Targeted Therapies: Refining Anticancer Therapy in the Era of Molecular Oncology. Clin Cancer Res, 15 (24): 7471-7478.
Reference 4—Justin N. Bottsford-Miller, Robert L. Coleman, and Anil K. Sood, 2012. Resistance and Escape From Antiangiogenesis Therapy: Clinical Implications and Future Strategies. J Clin Oncol, 30 (32): 4026-4034.
Reference 5—J P Hughes. S Rees. S B Kalindjian, and K L Philpott, 2011. Principles of early drug discovery. Br J Pharmacol, 162 (6): 1239-1249.
Reference 6—David H. Munn and Vincenzo Bronte, 2016. Immune suppressive mechanisms in the tumor microenvironment. Curr Opin Immunol, 39:1-6.
Reference 7—Pernick, N, 2018.
http://www.pathologyoutlines.com/topic/cdmarkerscd56.html
http://www.pathologyoutlines.com/topic/cdmarkerscd3.html
http://www.pathologyoutlines.com/topic/cdmarkerscd2.html
http://www.pathologyoutlines.com/topic/cdmarkerscd16.html
Reference 8—Zimmermann, J. Nicolaus. T. Neuert. G. and Blank, K, 2010. Thiol-based, site-specific and covalent immobilization of biomolecules for single-molecule experiments. Nat. Protoc. 5 (6): 975-985.

1. A method of treating a disease, comprising administering an effective amount of effector cells to a subject with the disease; the effector cells comprise a surface and a population of targeting units complexed to the surface of the effector cells; wherein a targeting unit in the population comprises a first ingredient; and the first ingredient is characterized in that: (a) it exhibits specific interaction with a biological marker expressed by abnormal cells associated with the disease; (b) it is not produced by the effector cell; and (c) it is determined to be ineffective or without adequate efficacy in treating the subject with the disease or concluded to be ineffective or without adequate efficacy in treating the disease at the end of a clinical trial.

2. A method of increasing the migratory capacity of immune cells into lesion of a disease, comprising administering an effective amount of effector cells to a subject with the disease; the effector cells comprise a surface and a population of targeting units complexed to the surface of the effector cells; wherein a targeting unit in the population comprises a first ingredient, and the first ingredient is characterized in that: (a) it exhibits specific interaction with a biological marker expressed by abnormal cells located in lesion of the disease; (b) it is not produced by the effector cell; and (c) it is determined to be ineffective or without adequate efficacy in treating the subject with the disease or concluded to be ineffective or without adequate efficacy in treating the disease at the end of a clinical trial.

3. An in vitro method of reducing the number of abnormal cells associated with a disease, comprising contacting a plurality of abnormal cells associated with the disease with an effective amount of effector cells; the cytotoxic cells comprise a surface and a population of targeting units complexed to the surface of the effector cells; wherein a targeting unit in the population comprises a first ingredient; and the first ingredient is characterized in that: (a) it exhibits specific interaction with a biological marker expressed by the abnormal cells associated with the disease; (b) it is not produced by the effector cell; and (c) it is determined to be ineffective or without adequate efficacy in treating the abnormal cells associated with the disease.

4. The method of claim 1, wherein the effector cells are cytotoxic cells.

5. The method of claim 1, wherein the effector cells comprise more than 3000 targeting units per cell.

6. The method of claim 1, wherein the effector cells are CD16⁺ cells.

7. The method of claim 1, wherein the first ingredient comprises an Fe receptor recognized region.

8. The method of claim 1, wherein the first ingredient is a monoclonal antibody of an IgG subtype that induces ADCC; or the first ingredient is other antibody; or the first ingredient comprises an antigen-binding unit.

9. The method of claim 1, wherein the first ingredient is not a nucleic acid.

10. The method of claim 1, wherein the effector cells are capable of mediating an antibody-dependent cell cytotoxicity (ADCC) response.

11. The method of claim 1, wherein the effector cells are capable of inducing the migration of CD3⁺ T cells.

12. The method of claim 1, wherein after co-cultured with target cells expressing the biological marker, the effector cells express CD107a.

13. The method of claim 1, wherein after co-cultured with target cells expressing the biological marker, the effector cells express interferon-γ (IFN-γ) or Tumor Necrosis Factor-α (TNF-α), or a combination thereof.

14. The method of claim 1, wherein the first ingredient is an FDA-approved ingredient for the treatment of the disease.

15. The method of claim 14, wherein the first ingredient is rituximab, trastuzumab, cetuximab, alemtuzumab, avelumab, daratumumab, elotuzumab, obinutuzumab, vorsetuzumab, cusatuzumab, durvalumab, panitumumab, or amatuzimab.

16. The method of claim 1, wherein the first ingredient has been successful in phase I clinical trial but is not an FDA-approved ingredient for the treatment of the disease.

17. The method of claim 16, wherein the first ingredient is codrituzumab, solanezumab, bimagrumab, traklokinumab, or bococizumab.

18. The method of claim 1, wherein the effector cells to be administered to the subject are derived from autologous effector cells or allogeneic effector cells.

19. The method of claim 1, wherein the effector cells are administered without inducing cell expansion prior to administration.

20. The method of claim 1, wherein the disease is selected from the group consisting of hyperproliferative diseases, advanced stage disease, HIV or other viral infectious diseases, fungi infectious diseases, bacteria infectious diseases, protozoan infectious diseases, autoimmune diseases, neuronal diseases, hematopoietic cell-related diseases, metabolic syndromes, and pathogenic diseases.

21. The method of claim 20, wherein the disease is a hyperproliferative or advanced stage disease selected from the group consisting of solid tumors and liquid tumors.

22. The method of claim 21, wherein the first ingredient exhibits specific interaction with a biological marker selected from the group consisting of cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis (a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, and MAGE, or any combination thereof, or the first ingredient exhibits specific interaction with a cancer antigen selected from the group consisting of HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF (F3), nectin-4, FAP, GPC3, FGFR3, a killer-cell immunoglobulin-like receptors (KIRs), ROR1, ROR2, PD-1 (CD279), CTLA-4 (CD152), TIM-3 (HAVCR2), an immune checkpoint receptor, an immune checkpoint receptor ligand, a receptor tyrosine kinase-like orphan receptor, a TNF receptor protein, an immunoglobulin protein, a cytokine receptor, an integrin, and activating NK cell receptors, and combinations thereof.

23. The method of claim 1, wherein the targeting unit is complexed to the surface of the effector cell via an interaction between a first linker conjugated to the first ingredient and a second linker conjugated to the surface of the effector cell.

24. The method of claim 23, wherein the first linker is covalently or non-covalently conjugated to the first ingredient; or the second linker is covalently or non-covalently conjugated to the surface of the effector cell; or a combination thereof.

25. The method of claim 23, wherein the first linker or the second linker is conjugated to a native functional group of the first ingredient or the surface of the effector cell, wherein the native functional group is an amino acid, a sugar, or an amine.

26. The method of claim 25, wherein the native functional group comprises a sugar, an amine, or an amino acid; or wherein the native functional group is not an azide-modified sugar; or wherein the native functional group comprises an amino acid selected from the group consisting of lysine, cysteine, tyrosine, threonine, serine, aspartic acid, glutamic acid and tryptophan.

27. The method of claim 25, wherein the second linker is directly, covalently linked to the native functional group of the effector cell; wherein the direct, covalent link between the second linker and the native functional group of effector cell is prepared by contacting the effector cell with the second linker, such that the second linker is directly, covalently linked to the native functional group.

28. The method of claim 23, wherein the first linker and the second linker are selected from the group consisting of: a DNA binding domain and a target DNA; a leucine zipper and a target DNA; biotin and avidin; biotin and streptavidin; calmodulin-binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

29. The method of claim 23, wherein at least one of the two linkers comprises a PEG region or an NHS ester; or wherein the first ingredient is conjugated to the first linker via a coupling group, wherein the coupling group is an NHS ester or other activated ester, an alkyl or acyl halide, a bifunctional crosslinker, or maleimide group.

30. The method of claim 1, wherein the first ingredient and the effector cell are separated by a length of 1 nm to 400 nm.

31. The method of claim 23, wherein the first linker is a first polynucleotide, and the second linker is a second polynucleotide.

32. The method of claim 31, wherein the length of at least one of the two polynucleotides is 4 nt to 500 nt.

33. The method of claim 31, wherein the first polynucleotide comprises a first single-stranded region, and the second polynucleotide comprises a second single-stranded region complementary to the first single-stranded region, wherein the targeting unit is complexed to the surface of the effector cell via the interaction between the first single-stranded region and the second single-stranded region complementary to the first single-stranded region.

34. The method of claim 23, wherein the first linker comprises a first reactive group, and the second linker comprises a second reactive group, and wherein the targeting unit is complexed to the surface of the effector cell via a covalent bond formed by a reaction between the second reactive group and the first reactive group.

35. The method of claim 4, wherein the cytotoxic cell is an immune cell, a lymphocyte, a natural killer cell, a gamma delta T cell, other T lymphocytes, macrophages, monocytes, a neutrophil, dendritic cells, cytokine-induced killer cells (CIK), lymphokine-activated killer cells (LAK), cytolytic T cells (CTLs), or tumor-infiltrating lymphocytes (TIL).

36. The method of claim 1, wherein the effector cell is non-tumorigenic in an immune compromised mouse; or the effector cell is non-tumorigenic in an allogeneic subject after being irradiated with γ-ray.

37. The method of claim 4, wherein the cytotoxic cell is a natural killer cell characterized in that:
(A) it is deposited at NPMD having the deposit number NITE BP-03017;
(B) it comprises a chromosome, and the chromosomal DNA sequence of the chromosome is at least 80% identical with the chromosomal DNA sequence of a corresponding chromosome of the natural killer cell deposited at NPMD having the deposit number NITE BP-03017; or
(C) it has the following characteristics:
i) expressing a CD16 receptor;
ii) retaining its capability to proliferate after subculture for at least 3 months; and
iii) x) not including synthetic, genetically modified and/or deliberately delivered polynucleotide encoding the CD16 receptor, or y) by using ddPCR system to analyze the genomic DNA of the cytotoxic cell, the ratio of CD16 F176F probe-detectable DNA molecule to CD16 F176V probe-detectable DNA molecule is equal to or higher than 1, wherein the sequence of the CD16 F176F probe is SEQ ID NO: 27 and the sequence of the CD16 F176V probe is SEQ ID NO: 28.

38. The method of claim 37, wherein the cytotoxic cell further characterized in that:
(1) the cytotoxic cell and the natural killer cell line NK3.3 are derived from different subjects;
(2) the cytotoxic cell is derived from a subject with a cancer;
(3) the cytotoxic cell is derived from a Caucasian male; or
(4) the cytotoxic cell and the natural killer cell having the deposit number ATCC CRL-2407 are derived from the same subject;
or any combination thereof.

39. The method of any one of chains 1 to 38 claim 1, wherein the effector cells further express CD2, CD45, CD4, CD25, NKp30, NKG2D, NKp44, NKp46, CD27, OX40, CD107a, NKG2A, PD-1, TIGIT, SIRPα, or CD158, or any combination thereof.

40. The method of claim 4, wherein the cytotoxic cell is a γδ T cell; or the cytotoxic cell is a Vδ1 T cell, a Vδ2 T cell, a Vδ3 T cell, a Vδ5 T cell, or a Vγ9Vδ2 T cell.

41. The method of claim 1, wherein the effector cells further express CD3, NKp46, CD56, CD16, NKG2D, NKp44, CD25, CD38, PD-1, NKp30, CD18, TIGIT, DNAM-1, CD36, CD103, CCR7, CXCR3, IFNγ, Granzyme B, or CD69, or any combination thereof.

42. The method of claim 1, wherein after co-culture with target cells expressing the biological marker, the effector cells further express Granzyme B.

43. The method of claim 1, wherein:
(1) at least 4% of the effector cells express at least 400 NKp46 molecules per cell;
(2) at least 10% of the effector cells express at least 400 CD56 molecules per cell;
(3) at least 10% of the effector cells express at least 400 CD16 molecules per cell;
(4) at least 30% of the effector cells express at least 40 NKG2D molecules per cell;
(5) at least 1% of the effector cells express at least 400 NKp44 molecules per cell;
(6) at least 80% of the effector cells express at least 400 CD69 molecules per cell; or
(7) at least 40% of the effector cells express at least 400 CXCR3 molecules per cell;
or any combination thereof.

44. The method of claim 1, wherein:
(1) at least 4% of the effector cells express NKp46, wherein the NKp46-expressing effector cells express at least 400 NKp46 molecules per cell on average;
(2) at least 10% of the effector cells express CD56, wherein the CD56-expressing effector cells express at least 400 CD56 molecules per cell on average;
(3) at least 10% of the effector cells express CD16, wherein the CD16-expressing effector cells express at least 400 CD16 molecules per cell on average;
(4) at least 30% of the effector cells express NKG2D, wherein the NKG2D-expressing effector cells express at least 40 NKG2D molecules per cell on average;
(5) at least 1% of the effector cells express NKp44, wherein the NKp44-expressing effector cells express at least 400 NKp44 molecules per cell on average;
(6) at least 80% of the effector cells express CD69, wherein the CD69-expressing effector cells express at least 400 CD69 molecules per cell on average; or
(7) at least 40% of the effector cells express CXCR3, wherein the CXCR3-expressing effector cells express at least 400 CXCR3 molecules per cell on average;
or any combination thereof.

45. The method of claim 1, wherein the targeting unit is a first type of targeting unit, and the effector cells further comprise a population of second type of targeting units complexed to the surface of the effector cells, wherein a targeting unit in the population of second type of targeting units comprises a second ingredient characterized in that (a) it exhibits specific binding to the biological marker or a different biological marker expressed by the abnormal cells associated with the disease; (b) it is not produced by the effector cell.


SEQUENCE LISTING (34 sequences)

SEQ ID NO: 1 - DNA, 20 bp, synthetic construct
  misc_feature 1..20: note = Synthetic
  source 1..20: mol_type = other DNA
cacacacacacacacacaca

SEQ ID NO: 2 - DNA, 20 bp, synthetic construct
  misc_feature 1..20: note = Synthetic
  source 1..20: mol_type = other DNA
tcatacgactcactctaggg

SEQ ID NO: 3 - DNA, 40 bp, synthetic construct
  misc_feature 1..40: note = synthetic
  source 1..40: mol_type = other DNA
actgatggtaatctgcacctactgatggtaatctgcacct

SEQ ID NO: 4 - DNA, 20 bp, synthetic construct
  misc_feature 1..20: note = Synthetic
  source 1..20: mol_type = other DNA
tgtgtgtgtgtgtgtgtgtg

SEQ ID NO: 5 - DNA, 20 bp, synthetic construct
  misc_feature 1..20: note = Synthetic
  source 1..20: mol_type = other DNA
ccctagagtgagtcgtatga

SEQ ID NO: 6 - DNA, 40 bp, synthetic construct
  misc_feature 1..40: note = synthetic
  source 1..40: mol_type = other DNA
aggtgcagattaccatcagtaggtgcagattaccatcagt

SEQ ID NO: 7 - DNA, 20 bp, synthetic construct
  misc_feature 1..20: note = synthetic
  source 1..20: mol_type = other DNA
aaaaaaaaaaaaaaaaaaaa

SEQ ID NO: 8 - DNA, 20 bp, synthetic construct
  misc_feature 1..20: note = synthetic
  source 1..20: mol_type = other DNA
tttttttttttttttttttt

SEQ ID NO: 9 - DNA, 20 bp, synthetic construct
  misc_feature 1..20: note = synthetic
  source 1..20: mol_type = other DNA
actgactgactgactgactg

SEQ ID NO: 10 - DNA, 20 bp, synthetic construct
  misc_feature 1..20: note = synthetic
  source 1..20: mol_type = other DNA
cagtcagtcagtcagtcagt

SEQ ID NO: 11 - DNA, 20 bp, synthetic construct
  misc_feature 1..20: note = synthetic
  source 1..20: mol_type = other DNA
gtaacgatccagctgtcact

SEQ ID NO: 12 - DNA, 20 bp, synthetic construct
  misc_feature 1..20: note = synthetic
  source 1..20: mol_type = other DNA
agtgacagctggatcgttac

SEQ ID NO: 13 - DNA, 20 bp, synthetic construct
  misc_feature 1..20: note = synthetic
  source 1..20: mol_type = other DNA
actgatggtaatctgcacct

SEQ ID NO: 14 - DNA, 20 bp, synthetic construct
  misc_feature 1..20: note = synthetic
  source 1..20: mol_type = other DNA
aggtgcagattaccatcagt

SEQ ID NO: 15 - DNA, 40 bp, synthetic construct
  misc_feature 1..40: note = synthetic
  source 1..40: mol_type = other DNA
aaaaaaaaaaaaaaaaaaaaaaaaaaaaaaaaaaaaaaaa

SEQ ID NO: 16 - DNA, 40 bp, synthetic construct
  misc_feature 1..40: note = synthetic
  source 1..40: mol_type = other DNA
tttttttttttttttttttttttttttttttttttttttt

SEQ ID NO: 17 - DNA, 40 bp, synthetic construct
  misc_feature 1..40: note = synthetic
  source 1..40: mol_type = other DNA
actgactgactgactgactgactgactgactgactgactg

SEQ ID NO: 18 - DNA, 40 bp, synthetic construct
  misc_feature 1..40: note = synthetic
  source 1..40: mol_type = other DNA
cagtcagtcagtcagtcagtcagtcagtcagtcagtcagt

SEQ ID NO: 19 - DNA, 40 bp, synthetic construct
  misc_feature 1..40: note = synthetic
  source 1..40: mol_type = other DNA
tgtgtgtgtgtgtgtgtgtgtgtgtgtgtgtgtgtgtgtg

SEQ ID NO: 20 - DNA, 40 bp, synthetic construct
  misc_feature 1..40: note = synthetic
  source 1..40: mol_type = other DNA
cacacacacacacacacacacacacacacacacacacaca

SEQ ID NO: 21 - DNA, 40 bp, synthetic construct
  misc_feature 1..40: note = synthetic
  source 1..40: mol_type = other DNA
gtaacgatccagctgtcactgtaacgatccagctgtcact

SEQ ID NO: 22 - DNA, 40 bp, synthetic construct
  misc_feature 1..40: note = synthetic
  source 1..40: mol_type = other DNA
agtgacagctggatcgttacagtgacagctggatcgttac

SEQ ID NO: 23 - DNA, 40 bp, synthetic construct
  misc_feature 1..40: note = synthetic
  source 1..40: mol_type = other DNA
tcatacgactcactctagggtcatacgactcactctaggg

SEQ ID NO: 24 - DNA, 40 bp, synthetic construct
  misc_feature 1..40: note = synthetic
  source 1..40: mol_type = other DNA
ccctagagtgagtcgtatgaccctagagtgagtcgtatga

SEQ ID NO: 25 - DNA, 23 bp, synthetic construct
  misc_feature 1..23: note = Synthetic
  source 1..23: mol_type = other DNA
agttaccatgacgtcaatttcag

SEQ ID NO: 26 - DNA, 23 bp, synthetic construct
  misc_feature 1..23: note = Synthetic
  source 1..23: mol_type = other DNA
ctgaaattgacgtcatggtaact

SEQ ID NO: 27 - DNA, 51 bp, synthetic construct
  misc_feature 1..51: note = synthetic
  source 1..51: mol_type = other DNA
tctgaagacacatttttactcccaaaaagccccctgcagaagtaggagccg

SEQ ID NO: 28 - DNA, 51 bp, synthetic construct
  misc_feature 1..51: note = synthetic
  source 1..51: mol_type = other DNA
tctgaagacacatttttactcccaacaagccccctgcagaagtaggagccg

SEQ ID NO: 29 - DNA, 2137 bp, Homo sapiens
  source 1..2137: mol_type = genomic DNA
cttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - DNA, 820 bp, Homo sapiens
  source 1..820: mol_type = genomic DNA
cactccagtgtggcatcatgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcag

SEQ ID NO: 31 - DNA, 765 bp, Homo sapiens
  source 1..765: mol_type = genomic DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 32 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 33 - protein, 233 aa, Homo sapiens
MWQLLLPTALLLLVSAGMRTEDLPKAVVFLEPQWYSVLEKDSVTLKCQGAYSPEDNSTQWFHSESLISSQASSYFIDAATVNESGEYRCQTNLSTLSDPVQLEVHIGWLLLRAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 34 - protein, 80 aa, Homo sapiens
HSWKNTALHKVTYLQNGKGRKYFHHNSDFYIPKATLKDSGSYFCRGLFGSKNVSSETVNITITQGLAVSTISSFFPPGYQ